(19) United States
(12) Patent Application Publication
(10) Pub. No.: US 2026/0306561 A1
(43) Pub. Date: Oct. 8, 2026

(54) TREATMENT OF CANCER THERAPY-INDUCED ORAL MUCOSITIS

(71) Applicant: BACTOPHARMA GMBH, Bischofszell (CH)

(72) Inventors: Karsten BRANDT, Sitges (ES); Michael FABER, Sitges (ES); Pei Tzu LEE, New Taipei City (TW); Qiang WANG, Wuhan City, Hubei (CN)

(21) Appl. No.: 19/484,680

(22) PCT Filed: May 15, 2024
(86) PCT No.: PCT/EP2024/063407

§ 371 Date: Nov. 14, 2025

(30) Foreign Application Priority Data
May 15, 2023 (EP) 23173410.4

(51) Int. Cl. A61K 35/744; A61K 9/00; A61K 47/12; A61K 47/26; A61K 47/36; A61P 1/02

(52) U.S. Cl. CPC A61K 35/744; A61K 9/0056; A61K 47/12; A61K 47/26; A61K 47/36; A61P 1/02

(57) ABSTRACT

Provided are Streptococcus salivarius and compositions comprising Streptococcus salivarius for the treatment of an ENT disease, in particular of cancer therapy-induced oral mucositis and cancer therapy-associated respiratory tract infections.

### FIELD OF THE INVENTION

The present invention relates to Streptococcus salivarius for use in the treatment and prevention of ear, nose, and throat (ENT) diseases, wherein the ENT disease is induced by or associated with a cancer therapy, preferably wherein said ENT disease is oral mucositis or a respiratory tract infection. Furthermore, the present invention relates to the use of Streptococcus salivarius in the maintenance of a healthy oral mucosa, during or after cancer therapy and to Streptococcus salivarius for use in the treatment and prevention of a respiratory tract infection in an immunosuppressed patient.

### BACKGROUND OF THE INVENTION

Cancer is a global health problem responsible for one in six deaths worldwide. In 2020, there were an estimated 19.3 million new cancer cases and about 10 million cancer deaths globally.

Cancer treatment modalities can be divided into conventional (traditional) and advanced, novel or modern categories. In this era worldwide, over half of all ongoing medical treatment trials are focusing on cancer treatments. Entities, such as the type of cancer, its site, and severity, guide to select treatment options and its progress. The most widely used traditional treatment methods are surgery, chemotherapy, and radiotherapy, wherein chemotherapy is considered the most effective and widely used modality in treating cancers as used alone or in combination with radiotherapy, while modern modalities include hormone therapy, anti-angiogenic, stem cell therapies, including hematopoietic stem cell transplantation (HSCT), immunotherapy, and dendritic cell-based immunotherapy (Debela et al., SAGE Open Med. 9 (2021), doi: 10.1177/20503121211034366).

Chemotherapy and radiotherapy are widely used methods of non-surgical cancer treatments which prolong life or even completely treat the disease. However, they cause numerous toxic side effects, including oral mucosa disorders, which strongly impairs quality of life. The most widely observed disorder is oral mucositis. About 40% of the patients treated with chemotherapy develop mucositis; this percentage rises to about 90% for head and neck cancer patients (HNC) treated with both chemo- and radiotherapy (Kusiak et al., Int J Environ Res Public Health. 17 (2020), 2464; Pulito et al., J Exp Clin Cancer Res 9 (2020), 210).

Allogeneic HSCT is considered the most potent post-remission antileukemic therapy in adult acute lymphoblastic leukemia. However, acute oral complications including mucositis, local and systemic infections, in particular of the ear, nose, and throat (ENT) tract, oral dryness, and taste changes are frequently encountered (Haverman et al., Mediators Inflamm. (2014), 378281). In particular, 85% of patients receiving intensive chemotherapy for HSCT also develop oral mucositis, and the incidence of severe oral mucositis (≥Grade 3) is 100% when patients receive intensive therapy of total body irradiation (TBI), along with cyclophosphamide chemotherapy-combined HSCT.

Accordingly, oral mucositis (OM), characterized by inflammation and mucosal damage of oral mucosa, is the most common oral complication of patients receiving cancer therapy. OM can cause erythema, ulceration, pain, dysphagia, and malnutrition, seriously affecting the quality of life of patients and interrupting anti-cancer treatment. Severe oral mucositis might be the cause of unplanned hospitalization or even lead to treatment changes including breaks in radiation or reduction of chemotherapy dosage, resulting in negative effects on treatment outcome.

To better understand OM and find ways to prevent and treat it, the mechanism of the disease has been widely studied. Researchers have found OM to be much more complicated than originally thought. It was found that the development of mucositis was a dynamic process typically divided into five stages: initiation, primary damage response, signal amplification, ulceration and healing. In the initial stage, cancer treatment, like chemotherapy or radiotherapy cause both DNA and non-DNA damage to basal epithelial cells, and a cascade of reactive oxygen species (ROS) is triggered which directly damages cells, tissues, and blood vessels. In the primary damage response stage, mucositis is driven by three pathways activated by the ROS cascade: NF-κB pathway, the ceramide pathway and the matrix metalloproteinase pathway. In the signal amplification stage, a series of pro-inflammatory cytokines, including TNF-α, IL-1β and IL-6, further damage the basal epithelial cells through positive feedback in the three pathways. In the ulceration stage, the loss of mucosal integrity facilitates the colonization of bacteria, which stimulates macrophagocytes to produce more pro-inflammatory cytokines and potentiates tissue injury. This stage causes extreme pain in patients. Lastly, in the healing stage, signals from submucosal extracellular matrix and mesenchyme promote cell proliferation and differentiation to reestablish the mucosal barrier (Shu et al., Oral Oncology 102 (2020), 104559).

Although the mechanism is well studied, the management of OM is still challenging. Interventions including basic oral care, growth factors and cytokines, anti-inflammatory agents, cryotherapy, and low-level laser therapy are suggested according to Multinational Association of Supportive Care in Cancer/The International Society of Oral Oncology (MASCC/ISOO) Clinical Practice Guidelines. However, only palifermin (keratinocyte growth factor-1) has been approved by the US Food and Drug Administration and the European Medicines Agency to mitigate OM in a very limited segment of the at-risk population (Shu et al., Oral Oncology 102 (2020), 104559)

Accordingly, further strategies for the treatment of oral mucositis are of utmost importance for the well-being of patients during cancer treatment and for a successful treatment outcome.

### SUMMARY OF THE INVENTION

The present invention generally relates to Streptococcus salivarius for use in a method of treating or preventing an ear, nose, and throat (ENT) diseases indued by or associated with a cancer therapy, in particular oral mucositis (OM) induced by cancer therapy or a respiratory tract infection (RTi) associated with cancer therapy, wherein the method comprises administering to a subject in need thereof the probiotic microorganism Streptococcus salivarius. The present invention is based on the surprising finding that OM developed by cancer patients receiving radiotherapy and concurrent chemoradiation therapy (CCRT), respectively, or which underwent HSCT, was prevented, reduced and ameliorated in those patients which had been treated with the probiotic microorganism Streptococcus salivarius K12 before, concomitantly and/or after the cancer therapy and that administration of the probiotic microorganism Streptococcus salivarius K12 was safe in patients receiving cancer treatment and even reduced the incidence of respiratory tract infections in said patient group.

Hitherto, investigations of probiotics, e.g., Lactobacillus brevis, Lactobacillus lactis, and a mixture of Bifidobacterium longum, Lactobacillus lactis, and Enterococcus faecium for their potential to treat cancer therapy-induced OM have been reported, with mixed and therefore unreliable results; see review by Shu et al., Oral Oncology 102 (2020), 104559. In addition, the mechanism of the protective effect of probiotics against various kinds of conditions in general remained unclear.

Experiments performed within the scope of the present invention surprisingly revealed that a probiotic strain, i.e., Streptococcus salivarius K12 has a remarkable curative effect on the development of OM and reduces OM symptoms, when applied to patients receiving CCRT as shown in Example 1 or patients who underwent HSCT as shown in Example 2.

In particular, as can be seen in FIG. 1 and as described in detail in Example 1, during the 7 weeks of CCRT, the incidence of radiotherapy induced oral mucositis (RIOM) with RTOG (Radiation Therapy Oncology Group) score ≥2 was reduced in patients receiving the oral probiotic Streptococcus salivarius K12. Furthermore, there was a 3-week delay in the onset of RIOM with RTOG ≥2 for >60% of the patients, while patients taking oropharyngeal probiotic were fully protected from severe RIOM classified as RTOG 3 and 4 in contrast to patients not taking the probiotic, wherein a RTOG score of 4 is classified as life-threating and includes ulceration, hemorrhage, or necrosis. In contrast, a RTOG score of only 2 is classified as moderate.

The OM developed in patients receiving CCRT is classified as RIOM since in contrast to OM developed by mere chemotherapy (CIOM), radiation has next to a cytotoxic effect also an additional necrotic and inflammatory effect on the oral mucosa.

As shown in FIG. 6 and described in detail in Example 2, during a 130-day study of HSCT patients (HSCT is a therapy used for multiple malignant and nonmalignant diseases, with preceding chemotherapy used for pretransplantation myeloablation), the incidence of OM grade ≥2 (WHO grading scale) was reduced in patients who received the oropharyngeal probiotic Streptococcus salivarius K12 in comparison to the control group, which did not receive the probiotic. Remarkably, no ulcerative OM was observed in patients in the probiotic group during the 100 days of oropharyngeal probiotic intervention. Accordingly, thanks to the experiments performed in accordance with the present invention, for the first time a therapeutically effective probiotic based treatment of RIOM and OM induced by HSCT and by the preceding chemotherapy (CIOM), respectively, in a human subject could be established.

During the present studies described in Examples 1 and 2, the occurrence of adverse events (side reactions) was also monitored and in both studies, no adverse events were reported. Thus, Streptococcus salivarius is considered as safe in patients who have been treated with radiotherapy and underwent HSCT, respectively. This is particularly important since patients subjected to anti-cancer treatment are rendered immunosuppressed, and therefore additional caution and comprehensive safety evaluation are needed before the application of probiotics. Accordingly, for approval of a probiotic therapy on humans, especially humans who are immunocompromised, for example due to cancer treatment or treatment with other immunosuppressive drugs proof-of concept in clinical trials are indispensable.

Accordingly, the human studies performed in accordance with the present invention are an important step towards the approval of a probiotic strain, namely Streptococcus salivarius, for the treatment of cancer therapy-induced OM.

Further mouse studies have been performed to visualize the effect of chemotherapeutic agents and of Streptococcus salivarius on oral epithelial cells and thus, to monitor the efficiency of Streptococcus salivarius in the treatment of chemotherapy induced OM. The anti-cancer drug, here chemotherapy (busulfan in combination with cyclophosphamide (Example 3) and 5-fluorouracil (Example 4)) has been administered over a time span of five to six days to mimic the repeated administration in human subjects and the development of OM. This mode of administration is indeed similar to the mode of administration performed in human patients (in contrast to radiotherapy which is usually administered over a period of several weeks, which makes it difficult to establish a suitable mouse model to study RIOM) since chemotherapy is usually delivered over a short time, in which case the injury to mucosal tissues tends to be acute. Chemotherapy induced OM (CIOM) usually develops within 4-7 days after initiation of treatment and peaks within 2 weeks. This period can be well observed in mice.

While there is similarity in the cellular events of CIOM and RIOM, the biological pathways are slightly different and as mentioned above, radiation has next to a cytotoxic effect an additional necrotic and inflammatory effect on the oral mucosa. In particular, chemotherapy is administered systemically, whereas radiation therapy affects a specific body area. In addition, as mentioned above, there are differences in the kinetics of treatment, affecting the clinical course. Chemotherapy may be delivered over a short time, in which case the injury to mucosal tissues tends to be acute. CIOM usually develops within 4-7 days after initiation of treatment and peaks within 2 weeks. Radiotherapy has a more gradual clinical course since it is most often administered in small fractions given over weeks. RIOM typically begins at cumulative doses of about 15 Gy (after around 10 days) and typically reaches full severity at 30 Gy, lasts for weeks or even months (Raber-Durlacher et al., Oral Oncology 46 (2010) 452-456).

As described in Example 3 and shown in FIGS. 10, 11 and 12, Streptococcus salivarius K12 promotes the healing of CIOM in mice. In particular, chemotherapy treatment in mice led to conspicuous mucosal hypoplasia and ulceration in the tongue. Furthermore, the basal layer cells were only loosely aligned and pyknosis, an irreversible condensation of chromatin in the nucleus of a cell undergoing necrosis or apoptosis, was observed. In addition, the tongue tissue had less stratum spinosum and granular layer cell than healthy tissue; see FIGS. 10A and 10B. Treatment with Streptococcus salivarius K12 during chemotherapy restored the integrity of the lingual mucosa and partially restored the basal layer, stratum spinosum and granular layer; see FIG. 10C. It was further observed that chemotherapy treatment reduced the mucosal thickness by nearly 50% and that the reduction was less, i.e., about 25% when Streptococcus salivarius K12 was administered during chemotherapy; see FIGS. 11 A to C, and FIG. 12. Same was observed after treatment of mice with 5-Fu. As shown in Example 4 and FIG. 17, the area of oral mucosal layer of mice was reduced by 5-Fu treatment and significantly increased by S. salivarius K12 treatment.

Based on these experiments, the inventors had the idea that inactivated Streptococcus salivarius K12 might have a beneficial effect on the prevention and treatment of OM. This idea was developed on the fact that inactivated probiotics were shown to reduce system inflammations and retained the cellular and molecular biological reactive properties of the viable probiotic (WO 2008/106373 A1). Furthermore, a study confirmed that heat-inactivated Lactococcus salivarius CECT 5713 prevents Streptococcus mutans adhesion to hydroxyapatite and thus, the inactivated from might be used as a strategy to reduce the salivary concentration of this oral pathogen (Sanudo et al., Archives of Oral Biology 84 (2017), 58-63). For S. salivarius it has also been shown that the cell wall comprises antigenic compounds (Montague and Knox, J Gen Microbiol. 54 (1986), 237-246); Weerkamp and Jacobs, Infection and Immunity 38 (1982), 233-242), and thus the inventors developed the invention that cell wall components could be sufficient to elicit an immune response. This was completely against the teaching in the prior art in the field of the treatment of oral mucositis. For example, in Wang et al., Front. Immunol. 12 (2021), 684824, the effect of S. salivarius on the treatment of RIOM was attributed to the ability of live S. salivarius to reconstitute the oral microbiota and thus, based on this publication, the skilled person would not have tried to use inactivated cells.

As shown in Example 4, treatment of oral epithelial cells with heat-inactivated S. salivarius K12 indeed restored their proliferation capacity after it was decreased upon 5-Fu treatment; see FIG. 16. Furthermore, the oral mucosal barrier was protected by heat-inactivated S. salivarius K12. As shown in FIG. 18, the area of oral mucosal layer of mice was reduced by 5-Fu treatment and significantly increased by heat-inactivated S. salivarius K12. Same effect was observed with live S. salivarius K12 (see FIG. 17), which was already shown in clinical studies to be suitable for the treatment of oral mucositis (see e.g., Example 2). Thus, both live and heat-killed S. salivarius K12_significantly promote the proliferation of oral mucosa cells and accordingly, both are suitable for the treatment of oral mucositis.

Accordingly, the present invention also relates to the use of inactivated S. salivarius K12 for use in the treatment of an oral mucosa disorder, in particular of oral mucositis, and most preferably in CIOM.

OM is not the only complication which might occur during cancer treatment. Due to the immunosuppressive effect of the anti-cancer drugs, in particular of chemotherapy and HSCT, infections of the respiratory tract are quite common. Previous clinical studies in adults and children have already established the efficacy of daily administration of Streptococcus salivarius in reducing respiratory tract infections and respiratory key pathogens.

In the study described in Example 2, the effect of S. salivarius K12 on respiratory tract infections in HSCT-patients, and thus patients with an impaired immune system, was analyzed. It was shown that the adjuvant treatment with S. salivarius K12 can effectively reduce the prevalence of RTi in patients post-HSCT, shorten the duration of respiratory symptoms, and reduce the days of antibiotic use. The latter observation is particularly important since the use of antibiotics in HSCT recipients in controversial discussed and it has been reported that the use of antibiotics confers a long-term adverse effect on overall survival post-HSCT.

Accordingly, since on the one hand, the safety of Streptococcus salivarius K12 has been proven for immunosuppressed patients for the first time during the studies performed within the scope of the present invention and since on the other hand, the therapeutic effect on respiratory tract infections in HSCT-patients has been shown, Streptococcus salivarius can be used for the treatment of ENT diseases induced by cancer therapy, and in particular ENT disease induced by chemotherapy and/or radiotherapy as well as in patients who received HSCT, and in particular patients who are immunosuppressed.

### BRIEF DESCRIPTION OF THE DRAWINGS

FIG. 1: Oropharyngeal probiotic Streptococcus salivarius (ENT-K12) prevents severe RIOM in nasopharyngeal carcinoma patients during CCRT treatment. The diagram shows the mean RTOG score that the patients experienced during the 7 weeks of CCRT, wherein the onset of RIOM (RTOG=1) started to occur during the 2^{nd} and 3^{rd} week of the CCRT course for CCRT group (control group) and CCRT-P group (group that received the probiotic), respectively.

FIG. 2: Kaplan-Meier curve analysis for the first onset of RIOM with RTOG level I in the CCTR group (control group) and the CCRT-P group (group that received the oropharyngeal probiotic Streptococcus salivarius (ENT-K12)). The horizontal axis starts from the beginning of the CCRT course. A significantly faster decreased free rate of RIOM with RTOG I was observed in the CCRT group compared to those in the CCRT-P group (p<0.05). Y-axis: patients free from RIOM with RTOG I (%).

FIG. 3: Kaplan-Meier curve analysis for the first onset of RIOM with RTOG level II in the CCTR group (control group) and the CCRT-P group (group that received the oropharyngeal probiotic Streptococcus salivarius (ENT-K12)). The horizontal axis starts from the beginning of the CCRT course. A significantly faster decreased free rate of RIOM with RTOG II was observed in the CCRT group compared to those in the CCRT-P group (p<0.05). Y-axis: patients free from RIOM with RTOG II (%).

FIG. 4: Kaplan-Meier curve analysis for the first onset of RIOM with RTOG level III in the CCTR group (control group) and the CCRT-P group (group that received the oropharyngeal probiotic Streptococcus salivarius (ENT-K12)). The horizontal axis starts from the beginning of the CCRT course. A significantly faster decreased free rate of RIOM with RTOG III was observed in the CCRT group compared to those in the CCRT-P group (p<0.05). Y-axis: patients free from RIOM with RTOG III (%).

FIG. 5: Kaplan-Meier curve analysis for the first onset of RIOM with RTOG level IV in the CCTR group (control group) and the CCRT-P group (group that received the oropharyngeal probiotic Streptococcus salivarius (ENT-K12)). The horizontal axis starts from the beginning of the CCRT course, The free rate of RIOM with RTOG IV were not different between patients in the CCRT and CCRT-P group (p>0.05), as only 1 patient in the CCRT group developed RIOM with RTOG IV and the rest 9 patients did not. Y-axis: patients free from RIOM with RTOG IV (%).

FIG. 6: Oropharyngeal probiotic Streptococcus salivarius (ENT-K12) prevents severe OM in human subjects who underwent HSCT. The diagram shows the average event free rate (OM grade ≥2 according to the WHO grading scale) in the probiotic group (patients who received HSCT and the oropharyngeal probiotic ENT-K12) and in the control group (patients who received HSCT and did not receive the oropharyngeal probiotic ENT-K12). During the 130 days study, the incidence of OM grade ≥2 was reduced in patients who received the oropharyngeal probiotic.

FIG. 7: Probability of not having any episodes of RTi during the 130 days of study period, including 100 days of oropharyngeal probiotic intervention and 30 days of follow-up period. Kaplan-Meier analysis indicated that a constantly higher probability of not having RTi episode was observed in patients of probiotic group than that in the control group (p=0.071).

FIG. 8: Cumulative duration of presenting RTi-like symptoms during the 130 days of study period, including 100 days of oropharyngeal probiotic intervention and 30 days of follow-up period. Kaplan-Meier analysis indicated that a less cumulative duration of presenting RTi-like symptoms was constantly observed in the probiotic group (p=0.131).

FIG. 9: Cumulative duration of presenting RTi-like symptoms during the 130 days of study period, including 100 days of oropharyngeal probiotic intervention and 30 days of follow-up period. Kaplan-Meier analysis indicated that a less cumulative duration of presenting RTi-like symptoms was constantly observed in the probiotic group (p=0.131), resulting in a significantly less cumulative days of antibiotic consumption in the probiotic group during the whole study period (p=0.011).

FIG. 10: Representative images of H&E staining of dorsal tongues indicating the integrity of lingual mucosa (80×). A) control group (untreated mice); B) chemotherapy group (mice that received chemotherapy); C) chemotherapy+ENT-K12 group (mice that received chemotherapy and the oropharyngeal probiotic Streptococcus salivarius (ENT-K12)). In the chemotherapy group (B), conspicuous mucosal hypoplasia and ulceration in the tongue, and pyknosis, an irreversible condensation of chromatin in the nucleus of a cell undergoing necrosis or apoptosis, was observed, the basal layer cells were only loosely aligned and the tongue tissue had less stratum spinosum and granular layer cell than the control group (A). In the chemotherapy+ENT-K12 group (C), the integrity of the lingual mucosa was restored and the basal layer, stratum spinosum and granular layer were partially restored.

FIG. 11: Representative images of H&E staining of dorsal tongues showing the mucosal thickness (80×). A) control group (untreated mice); B) chemotherapy group (mice that received chemotherapy); C) chemotherapy+ENT-K12 group (mice that received chemotherapy and the oropharyngeal probiotic Streptococcus salivarius (ENT-K12)). In the chemotherapy group (B), the mucosal thickness was reduced by nearly 50% in comparison to the control group (A), and in the chemotherapy+ENT-K12 group (C), the reduction was about 25% in comparison to the control group (A).

FIG. 12: Diagram showing the mucosal thickness in longitudinal sections the mouse tongue. The mucosal thickness was reduced by nearly 50% in mice that received chemotherapy in comparison to untreated mice (healthy mucosa), and by only about 25% in mice that received chemotherapy and the oropharyngeal probiotic Streptococcus salivarius (ENT-K12).

FIG. 13: Live cell imaging experiment showed that the growth activity of oral epithelial cells (HOK cells) decreased after 48 hours of 5-Fu treatment.

FIG. 14: Proliferative activity of HOK cells was analyzed in a CCK-8 assay and the mitochondrial metabolic concentration was measured to reflect cell proliferation activity. A significantly decreased proliferation activity of HOK cells was induced by 5-Fu treatment with concentration >10 μg/mL for 48 hours (p<0.05).

FIG. 15: Proliferative activity of HOK cells was analyzed in a CCK-8 assay. Heat-killed Streptococcus salivarius ENT-K12 showed no proliferative toxicity to HOK cells at any concentration, and the cell viability of HOK was significantly increased by heat-killed Streptococcus salivarius ENT-K12 treatment when the concentration is >1.5 mg/mL.

FIG. 16: Proliferative activity of HOK cells was analyzed in a CCK-8 assay. The proliferation of HOK cells significantly decreased after 10 μg/mL 5-Fu treatment and was significantly and dose-dependently restored after heat-killed Streptococcus salivarius ENT-K12 treatment.

FIG. 17: Tissue slices (A-C) and a diagram showing the area of the oral mucosal layer (D) showed that 5-Fu had a significant adverse effect on the integrity and permeability of the oral mucosal barrier of mice. The area of oral mucosal layer of mice was reduced by 5-Fu treatment and significantly increased by live Streptococcus salivarius ENT-K12 treatment.

FIG. 18: Tissue slices (A-C) and a diagram showing the area of the oral mucosal layer (D) showed that 5-Fu had a significant adverse effect on the integrity and permeability of the oral mucosal barrier of mice. The area of oral mucosal layer of mice was reduced by 5-Fu treatment and significantly increased by heat-killed Streptococcus salivarius ENT-K12 treatment.

FIG. 19: Tissue slices (A-C) and a diagram showing the average fluorescence activity (D) showed that the expression of Ki-67 in the 5-Fu group was lower than that in the probiotic group (treatment with live Streptococcus salivarius ENT-K12) and the control group. Thus, live Streptococcus salivarius ENT-K12 treatment significantly promoted the proliferation of oral mucosa cells.

FIG. 20: Tissue slices (A-C) and a diagram showing the average fluorescence activity (D) showed that the expression of Ki-67 in the 5-Fu group was lower than that in the probiotic group (treatment with heat-killed Streptococcus salivarius ENT-K12) and the control group. Thus, heat-killed Streptococcus salivarius ENT-K12 treatment significantly promoted the proliferation of oral mucosa cells.

FIG. 21: Analysis of the cycle of HOK cells by flow cytometry. (A) Control; (B) HOK cells treated with heat-killed Streptococcus salivarius ENT-K12; (C) HOK cells treated with 5-Fu; (D) HOK cells treated with 5-Fu and heat-killed Streptococcus salivarius ENT-K12. Compared with the control group, heat-killed Streptococcus salivarius ENT-K12 did not significantly change the HOK cell cycle after 48 hr treatment. 5-Fu treatment induced the block of S phase and G2/M phase, which are associated with a period of rapid cell growth and protein synthesis during which the cell prepares itself for mitosis and cytokinesis. Heat-killed Streptococcus salivarius ENT-K12 restored the whole cell cycle after 5-Fu treatment.

FIG. 22: Reactive oxygen species (ROS) level of HOK cell was detected by flow cytometry. The ROS level of HOK cells was induced by 5-Fu treatment and was significantly reduced after the treatment of heat-killed S. salivarius ENT-K12. The FITC-A value indicates the reactive oxygen species (ROS) level of HOK cells reflecting the DNA damage during chemotherapy (the higher the value, the higher ROS level, the more damage was caused).

### DETAILED DESCRIPTION OF THE INVENTION

The present invention relates to the treatment of an ear, nose, and throat (ENT) disease in a human subject, wherein said ENT disease is preferably oral mucositis (OM), wherein the treatment comprises administration of Streptococcus salivarius. In particular, the present invention relates to Streptococcus salivarius or a composition comprising Streptococcus salivarius for use in the treatment of an ENT disease in a human subject, wherein the ENT disease is a cancer therapy-induced or cancer therapy-associated ENT disease, preferably wherein said ENT disease is cancer therapy-induced OM or a cancer-therapy associated respiratory tract infection (RTi). As regards the respiratory tract infection, the present invention relates in particular to its treatment in immunosuppressed patients, like in patients that received HSCT. The present invention also relates to the treatment of an oral mucosa disorder, in particular oral mucositis with inactivated Streptococcus salivarius. Furthermore, the present invention relates to the use of Streptococcus salivarius or a composition comprising Streptococcus salivarius in the maintenance of a healthy ear, nose, and throat (ENT) tract, preferably of a healthy oral mucosa, of a human subject during cancer therapy. Furthermore, the present invention relates to a tablet, in particular to a lozenge comprising Streptococcus salivarius for the indicated uses.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. The materials, methods, and examples are illustrative only and not intended to be limiting.

Other features and advantages of the invention will be apparent from the following detailed description and from the claims.

For the avoidance of any doubt it is emphasized that the expressions “in some embodiments”, “in a certain embodiments”, “in certain instances”, “in some instances”, “in a further embodiment”, “in one embodiment” and the like are used and meant such that any of the embodiments described therein are to be read with a mind to combine each of the features of those embodiments and that the disclosure has to be treated in the same way as if the combination of the features of those embodiments would be spelled out in one embodiment. The same is true for any combination of embodiments and features of the appended claims and illustrated in the Examples, which are also intended to be combined with features from corresponding embodiments disclosed in the description, wherein only for the sake of consistency and conciseness the embodiments are characterized by dependencies while in fact each embodiment and combination of features, which could be construed due to the (multiple) dependencies must be seen to be literally disclosed and not considered as a selection among different choices.

The terms “human subject” and “patient” are used interchangeable herein.

The term “treatment” refers to “therapeutic treatment” and “prophylactic treatment” and treatment of an ENT disease and treatment of OM also refers to patients which have not yet developed the ENT disease and OM, respectively, but who are at risk of developing such disease and which are susceptible to develop such disease, respectively. Patients who are at risk of developing the ENT disease and in particular the OM are for example patients who receive a cancer therapy and are otherwise immunosuppressed.

The terms “cancer treatment”, “anti-cancer therapy”, “cancer therapy”, and “anti-cancer drug” are used interchangeable herein. Furthermore, a cancer therapy can also be used to treat other diseases than cancer, for example autoimmune diseases. Accordingly, patients who receive an anti-cancer therapy must not necessarily suffer from cancer, but instead or in addition, for example, from an autoimmune disease. Accordingly, when reference is made to cancer therapy-induced OM, the OM can be induced by the treatment of cancer with a cancer therapy or by the treatment of another disease, in particular of an autoimmune disease, with a cancer therapy.

In more detail, HSCT, or treatment with chemotherapeutic agents or antimetabolites like methotrexate are not only treatment strategies for cancer patients, but HSCT is also a potentially curative treatment for patients with bone marrow failure syndromes, congenital immune deficiencies, or autoimmune diseases like multiple sclerosis, and low-dose chemotherapy and methotrexate treatment is also a potential treatment approach for autoimmune diseases, like rheumatoid arthritis, which also leads to OM as described above for the cancer patients (Haverman et al., Mediators Inflamm. (2014), 378281; Gobbo et al., Photon Lasers Med 2 (2013), 71-76; Gobbo et al., Photon Lasers Med 2 (2013), 71-76).

When reference is made to “cancer therapy-induced ENT disease”, “cancer therapy-induced oral mucositis”, “chemotherapy therapy-induced ENT disease”, chemotherapy therapy-induced OM (CIOM)”, “radiotherapy therapy-induced ENT disease”, radiotherapy therapy-induced OM (RIOM)”, “HSCT-induced ENT disease”, HSCT-induced OM”, or similar expressions, it means that the ENT disease and OM, respectively is induced by or associated with the mentioned therapy.

HSCT involves the administration of healthy hematopoietic stem cells to patients with dysfunctional or depleted bone marrow. This helps to augment bone marrow function and, depending on the disease being treated, leads to either destruction of malignant tumor cells or to generation of functional cells that can replace the dysfunctional ones, as is the case of immune-deficiency syndromes, hemoglobinopathies, and other diseases. HSCT is usually preceded by high-dose chemotherapy for pretransplantation myeloablation.

Streptococcus salivarius K12 is commercially available as a probiotic for more than 15 years. S. salivarius K12 is deposited with the Leibniz-Institut DSMZ—Deutsche Sammlung von Mikroorganismen und Zellkulturen GmbH, Inhoffenstraße 7B, 38124 Braunschweig, Germany under Accession No. DSM 13084 and is publicly available at the American Type Culture Collection (ATCC), P.O. Box 1549, Manassas, VA 20108, USA under Accession No. BAA-1024. S. salivarius ENT-K12 is genetically identical to S. salivarius K12 and has been deposited at the Leibniz-Institut DSMZ—Deutsche Sammlung von Mikroorganismen und Zellkulturen GmbH, Inhoffenstralße 7B, 38124 Braunschweig, Germany on Feb. 22, 2023, and assigned Accession Number DSM 34540. Streptococcus salivarius is also known as Streptococcus salivarius subsp. thermophilus ENT-K12 and thus, the terms “Streptococcus salivarius” “Streptococcus salivarius K12”, “Streptococcus salivarius ENT-K12”, “S. salivarius”, “S. salivarius K12”, “S. salivarius ENT-K12”, “Streptococcus thermophilus”, “Streptococcus thermophilus K12”, “Streptococcus thermophilus ENT-K12”, “S. thermophilus”, “S. thermophilus K12”, “S. thermophilus ENT-K12”, “S. salivarius subsp. thermophilus ENT-K12, “K12”, and “ENT-K12” are used interchangeable herein.

Thanks to the experiments performed within the scope of the present invention, it has been shown that administration of Streptococcus salivarius is efficient in the treatment of cancer therapy-induced OM. In particular, it has been shown that OM developed by cancer patients receiving radiotherapy and undergoing HSCT (preceded by chemotherapy), respectively, was prevented, reduced and ameliorated in patients which had been treated with the probiotic microorganism Streptococcus salivarius concomitantly with the cancer therapy. Furthermore, mice studies have confirmed the effect of Streptococcus salivarius in the treatment of chemotherapy-induced OM. The mice studies have also shown that inactivated, in particular heat-inactivated Streptococcus salivarius can be used for the treatment of OM. Furthermore, immune checkpoint inhibitor therapy is commonly used as cancer or autoimmune disease treatment, and it usually causes oral mucosal immune related adverse events, which is an important factor that could significantly affect the cancer/autoimmune disease treatment outcomes.

By virtue of their rapid mitotic rate, mucosal cells are natural targets of cancer cytotoxic regimens, wherein the cytotoxic therapy leads to epithelial damage, which is usually the onset of OM. Accordingly, in general, any cancer therapy can lead to OM and in particular cancer therapy based on cytotoxic drugs. Thus, a patient with any cancer or any other disease, which requires the administration of an anti-cancer drug, like an autoimmune disease, who develops OM due to cancer therapy is amendable for the treatment with Streptococcus salivarius. Furthermore, OM is not only associated with cancer therapy, but OM can also be associated with various other factors. For example, it is known that OM can occur in a variety of immunosuppressed patients (Chiappelli, Evid Based Complement Alternat Med. 2 (2005), 489-494).

Thus, in one aspect, the present invention relates to Streptococcus salivarius for use in the treatment of an oral mucosa disorder, preferably of OM, which is an ear, nose, and throat (ENT) disease, in a human subject. In a preferred embodiment, the OM is induced by or associated with cancer therapy. Accordingly, in the preferred embodiment, the patients to be treated with Streptococcus salivarius suffer from OM and a cancer disease or autoimmune disease and are undergoing cancer therapy, have completed cancer therapy, or are soon to be treated with a cancer therapy. Cancer therapies include but are not limited to radiotherapy, chemotherapy, chemoradiation therapy, in particular concurrent chemoradiation therapy, hormone therapy, anti-angiogenic stem cell therapy, including hematopoietic stem cell transplantation (HSCT), immunotherapy, dendritic cell-based immunotherapy, molecularly targeted therapy, proton pump inhibitor therapy, immune checkpoint inhibitor therapy, and hormone therapy.

In a preferred embodiment, the cancer therapy is a cancer therapy that is based on cytotoxic drugs. In other words, the OM which is preferably treated in accordance with the present invention is an anti-cancer drug-induced OM, for example a cytotoxic anti-cancer drug-induced OM.

In a preferred embodiment of the present invention, the cancer therapy which induces the OM is chemotherapy, radiotherapy, HSCT, immune checkpoint inhibitor therapy, a combination of chemotherapy and radiotherapy, a combination of HSCT and chemotherapy, a combination of HSCT and radiotherapy, or a combination of HSCT, chemotherapy and radiotherapy. Accordingly, the OM which is treated in accordance with the present invention is CIOM, RIOM, HSCT-induced OM, or any combination thereof as mentioned above.

Thus, in one embodiment, the present invention relates to Streptococcus salivarius for use in the treatment of OM in a human subject, wherein the OM is induced by chemotherapy, radiotherapy, HSCT, a combination of chemotherapy and radiotherapy, a combination of HSCT and chemotherapy, a combination of HSCT and radiotherapy, or a combination of HSCT, chemotherapy and radiotherapy. In other words, the patients to be treated with Streptococcus salivarius suffer from OM and a cancer disease or an autoimmune disease and are undergoing cancer therapy or have completed cancer therapy, wherein the cancer therapy is chemotherapy, radiotherapy, HSCT, a combination of chemotherapy and radiotherapy, a combination of HSCT and chemotherapy, a combination of HSCT and radiotherapy, or a combination of HSCT, chemotherapy and radiotherapy.

In one embodiment, the present invention relates to Streptococcus salivarius for use in the treatment of OM, preferably in a human subject, wherein the OM is CIOM.

The present invention further relates to Streptococcus salivarius for use in the treatment of OM, preferably in a human subject, wherein the OM is RIOM. As explained above, an OM is referred to as RIOM if the patient received either radiotherapy or chemoradiation therapy.

The present invention further relates to Streptococcus salivarius for use in the treatment of OM in a human subject, wherein the OM is HSCT-induced OM.

Chemotherapeutic agents are well known by the person skilled in the art and are listed in various publicly available lists, for example on the homepage of the Cancer Network “Chemotherapeutic Agents and Their Uses, Dosages, and Toxicities, Jun. 1, 2016, Anne M. McDonnell, PharmD, BCOP”. In one embodiment, the chemotherapy is a busulfan and cyclophosphamide combination chemotherapy. In another embodiment, the chemotherapy is 5-Fu chemotherapy.

As mentioned above, radiotherapy is usually applied over several weeks and suitable dosing can be determined by the skilled person in the art. As shown in Example 1, radiotherapy was administered 5 times per week at 2 Gy per fraction in a total dose of 60-70 Gy within 6-7 week to treat NPC. Accordingly, in a preferred embodiment, radiotherapy is applied 1 to 7 times per week, preferably 2 to 6 times per week, more preferably 3 to 6 times per week, more preferably 4 to 6 times per week, most preferably 5 times per week within 2 to 10 weeks, preferably within 3 to 10 weeks, more preferably within 4 to 10 weeks, more preferably 5 to 10 weeks, more preferably within 5 to 9 weeks, more preferably between 5 to 8 weeks, most preferably within 6 to 7 weeks. Preferably, the radiotherapy is administered at 2 Gy per fraction in a total dose of 60-70 Gy, but of course dependent on the disease to be treated, the treatment regimens can vary. In a preferred embodiment, the radiotherapy is administered with a concurrent chemotherapy. This mode of administration is used to treat nasopharyngeal carcinoma (NPC).

As shown in the studies performed within the scope of the present invention, the use of Streptococcus salivarius is efficient and safe in the treatment of OM in NPC patients as well as in patients which received HSCT; see Examples 1 and 2. NPC belongs to the neck and head cancers. Most head and neck cancers are derived from the mucosal epithelium in the oral cavity, pharynx and larynx and are known collectively as head and neck squamous cell carcinoma (HNSCC). However, HNSCC can arise from further areas within the head and neck including, but limited to: from the mucosal epithelium of the oral cavity (lips, buccal mucosa, hard palate, anterior tongue, floor of mouth and retromolar trigone), nasopharynx, oropharynx (palatine tonsils, lingual tonsils, base of tongue, soft palate, uvula and posterior pharyngeal wall), hypopharynx (the bottom part of the throat, extending from the hyoid bone to the cricoid cartilage) and larynx. Human papillomavirus-associated HNSCCs arise primarily from the palatine and lingual tonsils of the oropharynx, whereas tobacco-associated HNSCCs arise primarily in the oral cavity, hypopharynx and larynx. Accordingly, in one embodiment, the patient to be treated in accordance with the present invention suffers from a head and neck cancer, preferably NPC. Thus, in one embodiment, the OM to be treated in accordance with the present invention is caused by the treatment of a head and neck cancer, preferably NPC, with a cancer therapy.

In a preferred embodiment, the present invention relates to Streptococcus salivarius for use in the treatment of RIOM, wherein the cancer to be treated with radiotherapy is a head and neck cancer, preferably NPC. In a further preferred embodiment, radiotherapy is applied 5 times per week within 6 to 7 weeks (at 2 Gy per fraction in a total dose of 60-70 Gy).

HSCT is used for the treatment of a variety of diseases, including cancers, leukemia, lymphoma, cardiac failure, neural disorders, auto-immune diseases, immunodeficiency, metabolic or genetic disorders. The treatment with HSCT usually starts with a high-dose chemotherapy and thus, the OM developed in HSCT-patients can be referred to as CIOM. Thus, in one embodiment, the present invention relates to Streptococcus salivarius for use in the treatment of CIOM which is either developed due to chemotherapy treatment or due to chemotherapy treatment which is followed by HSCT. In a preferred embodiment, the present invention relates to Streptococcus salivarius for use in the treatment of CIOM, wherein the cancer to be treated with HSCT and preceding chemotherapy is a hematopoietic tumor.

The ability to consistently and accurately assess OM is critical to descriptions of its incidence and severity and in evaluating the effectiveness of potential interventions. There are three the most commonly used OM grading criteria (World Health Organization (WHO), Radiation Therapy Oncology Group (RTOG), and the common terminology criteria for adverse events (CTCAE). A comparison is provided in Villa et al., Support Care Cancer 29 (2021), 6061-6068. All grading criteria range from Grade 0 to Grade 4, wherein Grade 0 is defined as no finding, and Grade 4 represents the most severe form of OM. Accordingly, in one embodiment, the OM to be treated in accordance with the present invention can be of any grade between Grade 0 and Grade 4. In Examples 1 and 2 it has been shown that the incidence of RIOM with RTOG ≥2 was reduced and that patients taking oropharyngeal probiotic were fully protected from severe RIOM classified as RTOG 3 and 4. Furthermore, the administration of the oropharyngeal probiotic was shown to delay the onset of oral mucositis and reduces the risk of prevalence of severe oral mucositis, was shown to restore the decreased proliferation and the disturbed cell cycle of oral epithelial cells after cancer treatment in a dose-dependent manner, and was shown to prevent the reduction of the oral mucosal layer area and restores the disturbed integrity of the oral mucosa after chemotherapy treatment.

Accordingly, in one embodiment, S. salivarius is used in accordance with the present invention to prevent a patient from developing OM Grade ≥2. In one embodiment, S. salivarius is used in accordance with the present invention to prevent a patient from developing OM Grade 3 or 4. In one embodiment, S. salivarius is used in accordance with the present invention to prevent a patient from developing OM at all. In one embodiment, S. salivarius is used in accordance with the present invention to delay the onset of oral mucositis and to reduce the risk of prevalence of severe oral mucositis. In one embodiment, S. salivarius is used in accordance with the present invention to restore the decreased proliferation and the disturbed cell cycle of oral epithelial cells after chemotherapy treatment in a dose-dependent manner. In one embodiment, S. salivarius is used in accordance with the present invention to prevent the reduction of the oral mucosal layer area and restores the disturbed integrity of the oral mucosa after chemotherapy treatment.

It is further known that immunosuppression in general can lead to OM, for example due to due an HIV infection (Ehlert et al., Dtsch Med Wochenschr 138 (2013), 1601-1695), or treatment with immunosuppressive drugs. Since S. salivarius has been shown to be safe and efficient in the treatment of CIOM, RIOM and HSCT-induced OM in the experiments performed with the scope of the present invention, it is prudent to expect that OM developed in a patient due to its immunosuppressed state is also amendable to the treatment with S. salivarius. Accordingly, the present invention also relates to Streptococcus salivarius for use in the treatment of OM in a human subject, wherein the human subject is immunosuppressed. Immunosuppressants are commonly known in the art and are reviewed for example by Rathee et al., The Pharma Innovation Journal 1 (2013), 90-101.

As mentioned above, S. salivarius is a probiotic microorganism and in general, probiotics are defined as live microorganisms which when administered in adequate amounts confer a health benefit on the host. The beneficial effects of probiotics may be mediated by a direct antagonistic effect against specific groups of undesired organisms, resulting in a decrease of their numbers, by an effect on the metabolism of such groups of organisms or by a general stimulatory effect on the immune system of animal or human hosts.

As shown in Example 4, heat-inactivated Streptococcus salivarius has a similar beneficial effect on oral epithelial cells than live Streptococcus salivarius which has not shown before and thus, the present invention also relates to inactivated Streptococcus salivarius for use in the healing of cancer-therapy induced damaged oral epithelial cells, in particular of oral epithelial cells damaged due to chemotherapy. More particularly, the present invention also relates to inactivated Streptococcus salivarius for use in the treatment of cancer therapy-induced OM, and preferably in CIOM. Inactivation can be performed by various methods, for example by heat, pressure or radiation. Preferably, inactivation is performed by heat and thus, Streptococcus salivarius is preferably heat-inactivated (also referred to as heat-killed) Streptococcus salivarius. Heat inactivation is preferably performed with a temperature ranging von 60° C. to 120° C., more preferably at about 65° C. for about 60 min, or at about 120° C. for about 20 min.

As mentioned above, OM is an ENT disease. ENT diseases are also called otolaryngology conditions and include issues involving the ears, nose and nasopharynx, sinuses, throat, mouth and oropharynx, larynx, trachea, and the neck. The most common otolaryngology conditions are for example reviewed in Paradis and Messner, Pediatric Board Study Guide (2015), March 28, 469-89, which content is herein incorporated by reference. ENT diseases are usually caused by viral or bacterial infections.

Certain cancer therapies are known to temporarily weaken the immune system because they can cause a drop in the number of white blood cells made in the bone marrow. Accordingly, cancer treatment usually makes the patient more likely to get certain infections, for example the above-mentioned ENT diseases. Similar regards to immunosuppressive therapies in general. In the experiments performed within the scope of the present invention, it has been shown that administration of S. salivarius is safe for patients who are treated or have been treated with anti-cancer drugs and are thus immunosuppressed.

Furthermore, clinical studies have already established the safety and efficacy of daily administration of Streptococcus salivarius in reducing respiratory tract infections and respiratory key pathogens. In particular, it has been shown that strain K12 is well suited for use as an upper respiratory tract probiotic due to its natural propensity to inhabit the human oral cavity and be strongly competitive with a number of potential pathogens. In addition, it has been shown that S. salivarius K12 elicits an anti-inflammatory response. For example, S. salivarius K12 antagonizes the growth of S. pyogenes, the most important bacterial cause of pharyngeal infections in humans also affected by episodes of acute otitis media. Prophylactic administration of S. salivarius K12 to children with a history of recurrent oral streptococcal disease resulted in a considerable reduction of episodes of both streptococcal and viral infections; see Di Pierro et al., Drug Health Patient Saf. 6 (2014), 15-20. As outlined in detail in Example 2, it has also been verified in the preset study that Streptococcus salivarius can be used for the treatment and prevention of respiratory tract infections in patients that received a cancer treatment, in particular chemotherapy and HSCT.

Thus, the present invention also relates to Streptococcus salivarius not only for use in an oral mucosa disorder, but also for use in the treatment of further ENT diseases in a human subject, wherein the ENT disease is induced by cancer therapy. Accordingly, the patients to be treated with Streptococcus salivarius suffer from an ENT disease and a cancer disease or from an ENT disease and an autoimmune disease and are undergoing cancer therapy or have completed cancer therapy. Furthermore, the present invention relates to Streptococcus salivarius for use in the treatment of an ENT disease in a human subject, wherein the subject is immunosuppressed as explained above.

Since S. salivarius is particularly useful in the treatment of upper respiratory tract infections as confirmed by Example 2, and otitis media, the ENT disease is preferably an upper respiratory tract infection or otitis media. The upper respiratory tract includes throat, nose, pharynx, larynx, sinuses, and trachea and bacterial mediated upper respiratory tract infections include for example pharyngitis, tonsilitis, sinusitis, and bronchitis. Severe respiratory tract infections might be the cause of unplanned hospitalization or even lead to treatment changes including breaks in radiation or reduction of chemotherapy dosage, resulting in negative effects on treatment outcome. Furthermore, colonialization of the upper respiratory tract with S. pneumoniae can be the onset of lower respiratory tract colonialization which may lead to pneumonia, which can lead to death of patients, in particular of immunosuppressed patients. Accordingly, in a preferred embodiment, the ENT disease is a respiratory tract infection. Thus, the present invention relates in a preferred embodiment to Streptococcus salivarius for use in the prevention and treatment of respiratory tract infections during cancer therapy.

In particular, in one embodiment, the administration of Streptococcus salivarius during cancer therapy prevents the progression of an upper respiratory tract infection to a lower respiratory tract infection in the subject. In one embodiment, the administration of Streptococcus salivarius during cancer therapy shortens the duration of symptoms of a respiratory tract infection in the subject in comparison to a control, which did not administer Streptococcus salivarius. In one embodiment, the administration of Streptococcus salivarius during cancer therapy reduces the average duration of the respiratory tract infection episode in the subject in comparison to the control group. In one embodiment, the administration of Streptococcus salivarius during cancer therapy reduces the need of antibiotic consumption of the subject in comparison to the control group.

During the experiments made within the scope of the present invention it has thus been shown for the first time that Streptococcus salivarius can be used for the treatment of respiratory tract infections in patients undergoing cancer-therapy and thus in patients which are immunosuppressed. Before conduction of the present studies, it would not have been recommendable to administer live bacteria to already immunosuppressed patients since the risk of unwanted site effects, like a sepsis would have been too high. Accordingly, it has been shown for the first time that Streptococcus salivarius can be used for the treatment or an oral mucosa disorder, in particular OM, and a respiratory tract infection in patients receiving cancer therapy and in patients being immunosuppressed, respectively.

In a preferred embodiment, the cancer therapy comprises HSCT, chemotherapy, and/or radiotherapy.

As explained in detail above, during the experiments performed in accordance with the present invention, it has been further shown that severe OM was prevented in patients taking the oropharyngeal probiotic Accordingly, the administration of S. salivarius is useful for preventing OM and to maintain a healthy ear, nose, and throat (ENT) tract, preferably a healthy oral mucosa, of a human subject during cancer therapy. Thus, the present invention also relates to the use of Streptococcus salivarius in the maintenance of a healthy ear, nose, and throat (ENT) tract, preferably of a healthy oral mucosa, of a human subject during cancer therapy and of immunosuppressed patients, respectively.

The present invention further relates to compositions for use in accordance with the present invention, wherein the composition comprises as active ingredient Streptococcus salivarius (in an effective amount) and optionally one or more pharmaceutically acceptable excipients. The present invention also relates to compositions for use in in the treatment of a cancer-therapy induced oral mucosa disorder, in particular oral mucositis, wherein the composition comprises as active ingredient inactivated Streptococcus salivarius (in an effective amount) and optionally one or more pharmaceutically acceptable excipients.

A composition in accordance with the present invention is a composition appropriate for administration of S. salivarius to a patient in need of same, particularly a patient susceptible for the ENT disease, more particularly the OM as defined above and/or to a patient suffering from the ENT disease, in particular the OM. In general, therapeutic compositions are composed of S. salivarius and an acceptable excipient.

Excipients include bulking agents, diluents, carrier, binders, lubricants, disintegrators, anti-caking agents, preservatives, colors or flavors. The excipients are preferably pharmaceutically acceptable and/or acceptable for human consumption.

A carrier or bulking agent means a vehicle for delivery of the probiotic microorganism, to the individual, in which the vehicle is compatible with cell viability, or activity of the inactivated form of the probiotic, here S. salivarius. Acceptable carrier/bulking agents are well known to those skilled in the art; see, for example, Remington's Pharmaceutical Sciences, 18th ed., Gennaro, ed., 1990, Mack Publishing Co., Easton, Pa., incorporated herein by reference.

Suitable carriers are generally inert and can be either solid or liquid. Acceptable carriers include, but are not limited to, water, buffered saline solutions (e.g., phosphate-buffered saline), pharmaceutically acceptable culture media (e.g. BACa, TSBCaYE agar), or other solutions which maintain the viability of the bacterium and maintain the biologically active parts of the bacterium, respectively. Additionally, such pharmaceutically acceptable carriers may be aqueous or non-aqueous solutions, suspensions, and emulsions. A variety of pharmaceutically acceptable carriers suitable for oral, topical, nasal administration or administration through the respiratory tract of viable or lyophilized bacteria are well known in the art (see, for example, Remington's supra.). Suitable solid carriers known in the art include, for example, magnesium carbonate; magnesium stearate; celluloses; talc; sugars such as fructose, sucrose, mannitol, lactose; starches; flours; oligosaccharides and skim milk, and similar edible powders, but are not limited thereto. Carriers for administration of extracts are similarly well known.

Acceptable carriers/bulking agents suitable for use with probiotically active microorganisms in a compressed product like a lozenge are usually solid carriers known in the art and include, but are not limited to magnesium carbonate; magnesium stearate; celluloses; talc; sugars such as fructose, sucrose, mannitol, sorbitol, xylitol, lactose; sugar substitutes such as isomalt; starches; maltodextrin; flours; (fructose-)oligosaccharides and skim milk, and similar edible powders, but are not limited thereto.

Typical diluents, by way of example, are starches; lactose; mannitol; kaolin; calcium phosphate or sulphate; inorganic salts such as sodium chloride; and powdered sugars and sugar substitutes as mentioned above or celluloses.

Typical binders include starch; gelatin; sugars such as lactose, fructose, and glucose; and the like. Natural and synthetic gums are also convenient, including acacia; alginates; locust bean gum; methylcellulose, e.g., Hydroxypropyl methylcellulose (HMPC); polyvinylpyrrolidine (PVP) tragacanth; PVP K-30; PVP K-25; xanthan gum; and the like. Polyethylene glycol (PEG 4000 or PEG 6000); ethyl cellulose; and waxes can also serve as binders as well as Nu-BIND® and CompactCel®DIS.

Lubricants and anti-caking agents to prevent sticking during formulation and to prevent the formation of lumps include slippery solids such as talc, silica, magnesium and calcium stearate, polyethylene glycol, stearic acid, hydrogenated vegetable oils, rice extract blend, for example Nu-MAG®, CompactCel®LUB, potato starch, gum Arabica, CompactCel®FLO, Nu-FLOW®, silicon dioxide, tricalcium phosphate, and rice hulls, for example Nu-FLOW®. or CompactCel®FLO.

Disintegrators are substances which swell when wetted to break up the composition and release the S. salivarius or extract. The disintegrators include starches; clays; celluloses; algins and gums; more particularly corn and potato starches; methylcellulose; agar; bentonite; wood cellulose; cation exchange resins; alginic acid; guar gum; citrus pulp; carboxymethylcellulose; powdered sponge; silica; and sodium lauryl sulfate.

Aromatizing agents are known to the person skilled in the art and can be of any kind which give a good (or at least a different taste) to the compressed product. Those flavors include, but are not limited to strawberry, mint, orange, banana, passionfruit, cocoa, menthol, yuzu, lemon and/or combinations thereof, preferably passionfruit, cocoa and menthol; yuzu and mint; orange and mint.

S. salivarius can be formulated in any of a variety of compositions suitable for oral, nasal, or topical administration or administration through the respiratory tract. In a preferred embodiment S. salivarius in formulated suitable for oral administration. The composition can be in liquid, solid or semisolid form, but in a preferred embodiment, the composition is in a solid form. In another preferred embodiment, the composition is an oil drop composition, wherein S. salivarius is formulated with an oil, preferably a vegetable oil, preferably in sunflower oil and in particular in sunflower (Helianthus annuus L., alpha-tocopherol) seeds oil.

S. salivarius can be administered in the form of a mouth cream, oral film, wound dressing, lotion, gel, ointment, solution, suspension, emulsion, powder, granules, drops, in particular oil drops, sachet, mouth wash, mouth rinse, toothpaste, dentifrice, spray, gargle, capsule, lozenge, syrup, floss, film, chewing gum, or chewable tablet but the forms are not limited thereto. Administration can be performed through the respiratory tract by a nebulizer, with or without propellants. Preferably administration is performed in a dosage form that remains for a long time in the oral cavity, for example in the form of a lozenge, a chewable tablet, a chewing gum, an oil suspension, a powder/sachet or spray. In a preferred embodiment, the composition comprising Streptococcus salivarius as used in accordance with the present invention is in an oral solid dosage form selected from a tablet, a capsule, a gel, a lozenge, a chewable tablet, and a powder, preferably wherein the oral composition is a lozenge or a powder, more preferably a lozenge.

In general, the amount of S. salivarius administered to the patient will be an amount of an active agent high enough to deliver the desired benefit, but low enough to avoid serious side effects.

In one embodiment, the daily dose of Streptococcus salivarius to be administered in accordance with the present invention is between 3×10⁶ and 4×10¹⁰ CFU, preferably between 3×10⁹ and 3×10¹⁰ CFU or between 4×10⁹ and 4×10¹⁰ CFU. The inactivated form of S. salivarius in preferably administered in a similar amount than viable S. salivarius, i.e., in an amount effective to provide between 3×10⁶ and 4×10¹⁰ cell equivalents, preferably between 3×10⁹ and 3×10¹⁰ cell equivalents or between 4×10⁹ and 4×10¹⁰ cell equivalents.

In one embodiment, the composition, preferably the solid dosage form, more preferably the tablet, most preferably the lozenge as used in accordance with the present invention comprises at least 1 mg Streptococcus salivarius and may comprise up to 1000 mg Streptococcus salivarius. In a preferred embodiment, the composition, preferably the solid dosage form, more preferably the tablet, most preferably the lozenge as used in accordance with the present invention comprises between 1 to 500 mg Streptococcus salivarius, preferably 5 mg to 160 mg Streptococcus salivarius, preferably between 10 mg and 120 mg, more preferably between 10 mg and 100 mg, or between 30 mg and 120 mg, more preferably 40 mg, 50 mg, 60 mg, 100 mg or 120 mg, and most preferably 50 mg Streptococcus salivarius.

In one embodiment, the composition, preferably the solid dosage form, more preferably the tablet, most preferably the lozenge as used in accordance with the present invention comprises 50 mg Streptococcus salivarius and 10⁶ to 10¹⁰ CFU, preferably 1×10⁹ to 10×10⁹ CFU, more preferably 6×10⁹ to 7×10⁹ CFU of Streptococcus salivarius, respectively, or the corresponding amount of cell equivalents of the inactivated form. Preferably, said composition, preferably said solid dosage form and most preferably the lozenge is administered to the human subject several times a day, preferably 3 or 4 times a day. In one embodiment, the composition is administered 3 times a day to a patient who has undergone HSCT and/or has been treated with chemotherapy, and 4 times a day to a patient who has been treated with radiotherapy and/or wherein the cancer is NPC.

In one embodiment, the composition as used in accordance with the present invention and, Streptococcus salivarius, respectively, is administered over a period of several days to several weeks, wherein the period varies dependent of the kind of cancer/autoimmune disease treatment and dependent of the kind of cancer therapy, which is administered, respectively. For example, as mentioned above, chemotherapy is usually delivered over a short time, in which case the injury to mucosal tissues tends to be acute. Chemotherapy induced OM usually develops within 4-7 days after initiation of treatment and peaks within 2 weeks. Accordingly, it might be sufficient to administer the composition for about 2 weeks to avoid the development of a severe OM. In contrast, radiotherapy is usually delivered over several weeks as also shown in Example 1 and thus, in one embodiment, the composition as used in accordance with the present invention is administered for several weeks, preferably for a period of about 7 weeks to the human subject which has been treated with radiotherapy and/or wherein the cancer is NPC. In a preferred embodiment, the composition is administered even before commencement of the cancer therapy as preventive measure. For example, in case of radiotherapy and CCRT treatment, respectively, the composition is administered 2 weeks before commencement of the treatment.

In a preferred embodiment, the composition is administered 4 times a day. It is further preferred that administration is performed after breakfast, lunch, dinner, and teeth cleaning (mouth wash or teeth brushing) before bed.

In another embodiment, the composition/Streptococcus salivarius as used in accordance with the present invention is administered for a period of about 100 days to the human subject which has undergone HSCT after their hematopoietic system is reconstituted which is defined as blood platelets >20*10⁹ cells/L and neutrophil >0.5*10⁹ cells/L after HSCT, preferably wherein the composition is administered 3 times a day. It is further preferred that administration is performed after breakfast, lunch, and teeth cleaning (mouth wash or teeth brushing) before bed.

Administration of S. salivarius can be performed before, during (concurrently with), and/or after cancer therapy and can also be administered on long term basis in immunosuppressed subjects. In a preferred embodiment, administration of S. salivarius in patients with RIOM or CIOM is at least performed during the cancer therapy and preferably also after termination of cancer therapy, preferably as long as the OM symptoms are still present and as long as the patient is still immunosuppressed. As regards HSCT-induced OM, S. salivarius is preferably administered after their hematopoietic system is reconstituted which is for example defined as blood platelets >20*10⁹ cells/L and neutrophil >0.5*10⁹ cells/L after HSC.

In one embodiment, the composition as used in accordance with the present invention comprises, next to Streptococcus salivarius, isomaltulose and in addition one or more excipients, preferably a lubricant and/or an anti-caking agent, and/or a binder, and/or an aromatizing agent, preferably wherein the lubricant is magnesium stearate or a natural substitute, wherein the anti-caking agent is silicon dioxide, tricalcium phosphate or a natural substitute, wherein the binder is HMPC or a natural substitute, and/or wherein the aromatizing agent is a flavor, preferably strawberry flavor, and optionally wherein the product further comprises one or more active ingredients, preferably vitamins, minerals and/or fructooligosaccharides, preferably wherein the vitamin is vitamin D3.

Natural lubricants are known in the art and are for example a crude fat-containing bean powder as described in WO 2013/165131 A1, a rice extract blend, for example the product Nu-MAG®, or the product CompactCel® LUB (CompactCel® F clear 290.02 LUB, Biogrund GmbH, Huenstetten, Germany), an oat fiber blend, for example the product CompactCel® LUB (CompactCel® F 200.28 LUB, Biogrund GmbH, Huenstetten, Germany) or further products, like potato starch, or gum Arabica. Natural anti-caking agents are also known in the art and are for example powdered cellulose, like JELUCEL®, native potato starch, inulin, rice fibers, or rice hulls, for example Nu-FLOW®, or CompactCel®FLO. Natural binders are for example CompactCel®DIS, NuBind®, or pregelatinized corn starch.

In one embodiment, the composition as used in accordance with the present invention is
(i) a compressed product, i.e., an oral solid dosage form and comprises, next to Streptococcus salivarius, isomaltulose and in addition a lubricant/an anti-caking agent, preferably wherein the lubricant/anti-caking agent is magnesium stearate or a natural substitute; an aromatizing agent, preferably wherein the aromatizing agent is a flavor, preferably strawberry flavor; and optionally a natural sweetener and/or one or more active ingredients, preferably a vitamin, preferably vitamin D3;
(ii) a compressed product, i.e., an oral solid dosage form and comprises, next to Streptococcus salivarius, isomaltulose and in addition a lubricant/an anti-caking agent, preferably wherein the lubricant/anti-caking agent is magnesium stearate or a natural substitute; a binder, preferably wherein the binder is hydroxypropyl methylcellulose (HMPC) or a natural substitute; an aromatizing agent, preferably wherein the aromatizing agent is a flavor, preferably strawberry flavor; and optionally a natural sweetener and/or one or more active ingredients, preferably a vitamin, preferably vitamin D3;
(iii) a powder product and comprises, next to Streptococcus salivarius, isomaltulose and in addition an anti-caking agent, preferably wherein the anti-caking agent is silicon dioxide, tricalcium phosphate or a natural substitute; an aromatizing agent, preferably wherein the aromatizing agent is a flavor, preferably strawberry flavor; fructooligosaccharides; and optionally a natural sweetener and/or one or more active ingredients, preferably a vitamin, preferably vitamin D3.

The natural substitute of magnesium stearate is preferably a rice extract blend, preferably comprising rice extract, microcrystalline cellulose, and sunflower oil refined, for example the product CompactCel® F clear 290.02 LUB, an oat fiber blend, preferably comprising oat fibers, microcrystalline cellulose and sunflower oil refined, for example the product CompactCel® F 200.28 LUB, Nu-MAG®, potato starch, gum Arabica, rice hulls, like Nu-FLOW®, or CompactCel® FLO, but most preferably a rice extract blend. The natural substitute of HMPC is preferably a natural binder like CompactCel®DIS, NuBind®, or pregelatinized corn starch, most preferably pregelatinized corn starch.

Accordingly, in one embodiment, the composition as used in accordance with the present invention is a compressed product, i.e., an oral solid dosage form and comprises, next to Streptococcus salivarius, isomaltulose and in addition further comprises a rice extract blend as lubricant/anti-caking agent, pregelatinized corn starch as binder, a flavor as aromatizing agent, preferably strawberry flavor, a natural sweetener, preferably stevia, and vitamin D3 as further active ingredient.

An exemplarily solid dosage form comprises the ingredients as shown in Tables 1 to 3.

TABLE 1
Ingredients of an exemplarity isomaltulose/probiotics lozenge.

| | | Amount | Percentage |
|---|---|---|---|
| | Function | (mg) | (%) |
| Streptococcus | Active ingredient/ | 50.0 | 6.17 |
| salivarius | probiotic | | |
| K12/ENT-K12 | microorganism | | |
| Vitamin D3 premix | Active ingredient | 3.2 | 0.40 |
| Isomaltulose | Bulking agent | 740.8 | 91.45 |
| Magnesium stearate | Anticaking agent, | 8.0 | 0.99 |
| (vegetable) | lubricant | | |
| Strawberry flavor | Flavor | 8.0 | 0.99 |
| Total | | 810 | 100 |

TABLE 2
Ingredients of an exemplarity isomaltulose/probiotics lozenge.

| | | Amount | Percentage |
|---|---|---|---|
| | Function | (mg) | (%) |
| Streptococcus | Active ingredient/ | 50.0 | 6.17 |
| salivarius | probiotic | | |
| K12/ENT-K12 | microorganism | | |
| Vitamin D3 premix | Active ingredient | 3.2 | 0.40 |
| Isomaltulose | Bulking agent | 740.06 | 91.36 |
| Hydroxypropyl- | Binder | 0.74 | 0.09 |
| methlycellulose | | | |
| Magnesium stearate | Anticaking agent, | 8.0 | 0.99 |
| (vegetable) | lubricant | | |
| Strawberry flavor | Flavor | 8.0 | 0.99 |
| Total | | 810 | 100 |

TABLE 3
Ingredients of an exemplarity isomaltulose/probiotics tablet.

| | | Amount | Percentage |
|---|---|---|---|
| | Function | (mg) | (%) |
| Streptococcus | Active ingredient/ | 50.0 | 6.17 |
| salivarius | probiotic | | |
| K12/ENT-K12 | microorganism | | |
| Vitamin D3 premix | Active ingredient | 2.0 | 0.24 |
| Isomaltulose | Bulking agent | 734.16 | 90.64 |
| Pregelatinized | Binder | 7.42 | 0.92 |
| corn starch | | | |
| CompactCel ® LUB | Anticaking agent, | 8.1 | 0.99 |
| | lubricant | | |
| Strawberry flavor | Flavor | 8.0 | 0.99 |
| Stevia | Natural sweetener | 0.32 | 0.04 |
| Total | | 810 | 100 |

Magnesium stearate can be substituted with a natural lubricant/anti-caking agent like a rice extract blend, for example the product Nu-MAG®, or the product CompactCel® LUB (CompactCel® F clear 290.02 LUB, Biogrund GmbH, Huenstetten, Germany), an oat fiber blend, for example the product CompactCel® LUB (CompactCel® F 200.28 LUB, Biogrund GmbH, Huenstetten, Germany) or further products, like potato starch, or gum Arabica, powdered cellulose, like JELUCEL®, native potato starch, inulin, rice fibers, or rice hulls, for example Nu-FLOW®, or CompactCel®FLO, but preferably with a rice extract blend, preferably with CompactCel® LUB, and/or HMPC can be substituted with a natural binder like CompactCel®DIS, NuBind®, or preferably pregelatinized corn starch.

The compressed product of the present invention may also comprise silicon dioxide, but this can also be substituted with a natural anti-caking agent like rice hulls, for example CompactCel®FLO, or Nu-FLOW®. Preferably, the compressed product does not comprise magnesium stearate and silicon dioxide, but instead a rice extract blend.

An exemplarily powder product comprises the ingredients as shown in Table 4.

TABLE 4
Ingredients of an exemplarity isomaltulose/probiotics powder.

| | | Amount | Percentage |
|---|---|---|---|
| | Function | (mg) | (%) |
| Streptococcus | Active ingredient/ | 50.0 | 3.33 |
| salivarius | probiotic | | |
| K12/ENT-K12 | microorganism | | |
| FOS | Active ingredient | 480 | 32 |
| Isomaltulose | Bulking agent | 945 | 63 |
| Silicon dioxide | Anticaking agent | 15 | 1.0 |
| Strawberry flavor | Flavor | 10 | 0.67 |
| Total | | 1500 | 100 |

Silicon dioxide can be substituted with tricalcium phosphate or a natural anti-caking agent, like rice hulls (Nu-Flow®), or CompactCel® FLO as explained above, and can also be completely omitted.

The composition is preferably produced by a method comprising the following steps:
(i) mixing S. salivarius with isomaltulose and with one or more further excipients and optionally with one or more further active ingredients; and
(ii) homogenizing the mixture; and
(ii) compressing the mixture to a compressed product, preferably wherein compression is performed with a pressure of 6 to 10 kN/compressed product, or filling the mixture into sachets, and optionally
(iii) analyzing the CFU/g amount and/or the water activity; and optionally the disintegration time, the average mass, the resistance to crushing, the appearance, the friability, and/or the water activity of the composition.

In a preferred embodiment, the step (i) is composed of the following steps:
(a) mixing the isomaltulose with one or more further excipients, preferably with a lubricant/anti-caking agent, preferably with magnesium stearate;
(b) spraying a binder solution on the mixed excipients, preferably wherein the binder solution is composed of water and a binder, preferably wherein the binder is HMPC, thereby forming wet granules;
(c) drying the granules, preferably at a temperature of 40±2° C. for about 30 minutes; (d) grinding the granules;
(e) adding S. salivarius to the grinded granules and optionally adding one or more further active ingredients.

For details as regards the composition and the method for its production, reference is made to European patent application EP 23 167 706.3, which content is herein incorporated by reference.

In another embodiment, the composition, in particular the solid dosage form, as used in accordance with the present invention comprises, next to Streptococcus salivarius, one or more excipients, preferably are one or more bulking agents, lubricants, and optionally anti-caking agents and/or aromatizing agents.

In one embodiment, the composition, in particular the solid dosage form as used in accordance with the present invention comprises fructose and maltodextrin (bulking agent), magnesium stearate or a natural substitute (lubricant), a flavor, preferably strawberry flavor (aromatizing agent), and optionally silicon dioxide or a natural substitute (anti-caking agent). The natural substitutes are the same as mentioned above.

Exemplarily compositions, in particular solid dosage forms are shown in Tables 5 and 6.

TABLE 5
Ingredients of an exemplarity probiotics lozenge

| | | Amount | Percentage |
|---|---|---|---|
| | Function | (mg) | (%) |
| Streptococcus | Active ingredient/ | 50.0 | 5.26 |
| salivarius | probiotic | | |
| K12/ENT-K12 | microorganism | | |
| Fructose | Bulking agent | 585 | 61.58 |
| Maltodextrin | Bulking agent | 275 | 28.95 |
| Silicon dioxide | Anti-caking agent | 15 | 1.58 |
| Magnesium Stearate | Lubricant | 15 | 1.58 |
| (vegetable) | | | |
| Strawberry flavor | Flavor | 10 | 1.05 |
| Total | | 950 | 100 |

TABLE 6
Ingredients of an exemplarity probiotics lozenge

| | | Amount | Percentage |
|---|---|---|---|
| | Function | (mg) | (%) |
| Streptococcus salivarius | Active ingredient / | 50.0 | 5 |
| K12/ENT-K12 | probiotic | | |
| | microorganism | | |
| Fructose | Bulking agent | 635 | 63.5 |
| Maltodextrin | Bulking agent | 290 | 29 |
| Magnesium Stearate | Lubricant, | 15 | 1.5 |
| (vegetable) | Anticaking agent | | |
| Strawberry flavor | Flavor | 10 | 1 |
| Total | | 950 | 100 |

The composition as used in accordance with the present invention, in particular the solid dosage form is preferably prepared by a “briquetting” method comprising the following steps:
(i) briquetting the excipients, preferably with a pressure of about 7.5 kN/compressed product;
(ii) fractionizing and grinding the briquettes;
(iii) adding S. salivarius to the grinded briquettes and mixing;
(iv) compressing the mixture to the compressed product, preferably with a pressure of about 4.8 kN/compressed product; and optionally
(vii) analyzing the disintegration time, the average mass, the resistance to crushing, the appearance, the friability, and/or the water activity of the compressed product.

In a preferred embodiment, the method further comprises the following steps:
(a) drying, weighting and sieving of the excipient(s) before step (i), preferably wherein the drying is performed in a fluid be granulator;
(b) mixing and homogenizing the excipient(s) after step (a) and before step (i); (c) weighting and sieving of S. salivarius before step (iii), preferably wherein sieving is performed through a 1 mm net; and/or
(d) homogenizing the mixture after step (iii) and before step (iv).

For details as regards the composition and the method for its production, reference is made to the international application PCT/EP2023/059635, which content is herein incorporated by reference.

In one embodiment, the composition, in particular the solid dosage form as used in accordance with the present invention comprises isomalt (bulking agent), magnesium stearate or a natural substitute (lubricant), a flavor, preferably strawberry flavor (aromatizing agent), and optionally silicon dioxide or a natural substitute (anti-caking agent). The natural substitutes are the same as mentioned above.

An exemplarily composition, in particular solid dosage forms is shown in Table 7.

TABLE 7
Ingredients of an exemplarity probiotics lozenge

| | | Amount | Percentage |
|---|---|---|---|
| | Function | (mg) | (%) |
| Streptococcus salivarius | Active ingredient / | 50.0 | 6.17 |
| K12/ENT-K12 | probiotic | | |
| | microorganism | | |
| Vitamin D3 premix | Active ingredient | 2.0 | 0.25 |
| Isomalt | Bulking agent | 742 | 91.60 |
| Magnesium stearate | Anticaking agent, | 8.0 | 0.99 |
| (vegetable) | lubricant | | |
| Strawberry flavor | Flavor | 8.0 | 0.99 |
| Total | | 810 | 100 |

In all lozenges, the concentration of vitamin D3 (cholecalciferol) in the premix is 100,000 IU/g (1 I.U.=0,025 mcg).

As mentioned above, the oral solid dosage form as used in accordance with the present invention is in a preferred embodiment a lozenge. Accordingly, the present invention further relates to a lozenge comprising Streptococcus salivarius for use in accordance with the present invention as well as to the use of the lozenge in accordance with the present invention. In a preferred embodiment, the lozenge comprises the ingredients as mentioned above with regard to the oral solid dosage form. Most preferably, the lozenge comprises the ingredients as listed in Table 6, i.e., Streptococcus salivarius, fructose, maltodextrin, magnesium stearate and a flavor, preferably strawberry flavor, and most preferably the lozenge comprises the ingredients in the amounts listed in Table 6.

In one embodiment, the lozenge as used in accordance with the present invention is produced by the “briquetting” method as described above.

In one embodiment, the lozenge as used in accordance with the present invention is sucked by the patient until it is fully dissolved and not chewed or directly swallowed, preferably wherein the patient is not allowed to drink or swallow anything for at least 2 hours following the administration of the lozenge. In a preferred embodiment, the disintegration time of the lozenge is at least 4 to 5 minutes, which ensures that Streptococcus salivarius can colonize the oral cavity of the patient. The determination of the disintegration time can be performed as described in Chapter 2.9.1 of the European Pharmacopoeia 6.0 by Council of Europe; 6th Edition; published on May 10, 2008; ISBN-10:9287160546; ISBN-13:978-9287160546.

The present invention further relates to S. salivarius and to a corresponding composition as defined above, in particular to the lozenge for use according to the present invention, wherein S. salivarius or the corresponding composition, in particular the lozenge is packed in a kit comprising instructions for administering S. salivarius or the corresponding composition, in particular the lozenge. In a preferred embodiment, the lozenge is packed in a blister.

In another preferred embodiment, the composition is an oil drop, which is a preferred and convenient administration form for patients that have already developed a painful OM. An exemplarily oil drop composition is composed of the probiotic S. salivarius K12, sunflower (Helianthus annuus L., alpha-tocopherol) seeds oil, and cholecalciferol oil (Vitamin D3, alpha-tocopherol, medium chain triglycerides).

The present invention further relates to a method of treatment a cancer therapy-induced ENT disease, preferably cancer therapy-induced OM in a patient as described above, wherein the method comprises administration of S. salivarius, of the composition comprising S. salivarius, or of the lozenge comprising S. salivarius as described above. The present invention further relates to a method of treatment an ENT disease, preferably OM in an immunosuppressed patient as described above, wherein the method comprises administration of S. salivarius, of the composition comprising S. salivarius, or of the lozenge comprising S. salivarius as described above.

In a preferred embodiment, the Streptococcus salivarius as used in accordance with the present invention is Streptococcus salivarius K12 (American Type Culture Collection (ATCC), P.O. Box 1549, Manassas, VA 20108, USA, Accession No. BAA-1024) and S. salivarius ENT-K12 (Leibniz-Institut DSMZ—Deutsche Sammlung von Mikroorganismen und Zellkulturen GmbH, Inhoffenstralße 7B, 38124 Braunschweig, Germany, Accession Number DSM 34540), respectively.

Several documents are cited throughout the text of this specification. The contents of all cited references (including literature references, issued patents, published patent applications as cited throughout this application including the background section and manufacturer's specifications, instructions, etc.) are hereby expressly incorporated by reference; however, there is no admission that any document cited is indeed prior art as to the present invention.

A more complete understanding can be obtained by reference to the following specific examples which are provided herein for purposes of illustration only and are not intended to limit the scope of the invention.

### EXAMPLES

### Example 1: Oropharyngeal Probiotic ENT-K12 Prevents Severe Radio-Induced Oral Mucositis in Nasopharyngeal Carcinoma Patients During Radiotherapy Treatment

Concurrent chemoradiation therapy (CCRT) is the standard treatment strategy for nasopharyngeal carcinoma (NPC), wherein studies have shown that 59.4% to 100% of patients receiving CCRT for head and neck cancer have experienced oral mucositis (OM) Radiotherapy-induced oral mucositis (RIOM) is one of the most frequent complications in head and neck cancer (HNC) patients undergoing CCRT. The main manifestation of RIOM is discomfort or pain in the mouth or throat, and RIOM can reduce the patient's tolerance to radiotherapy, leading to a reduction in the total dose of radiotherapy, and may even lead to suspension of radiotherapy (Marta et al., Radiother Oncol 110 (2014), 9-15; Gobbo et al., Lasers Med Sci 31 (2016), 471-9). Due to the occurrence of RIOM, the incidence and degree of anxiety and depression in NPC patients during radiotherapy increases. This also decreases quality of life (QoL) and treatment adherence. In addition to this, RIOM has many sequelae including pain, sore throat, dysphagia, decreased oral intake and systemic infections, often delaying or interrupting treatment due to RIOM, and interruptions in treatment may lead to decreased disease control and poor patient prognosis. This not only negatively affects the quality of life, but also has a huge negative impact on tumor control and patient survival (Triarico et al., Pathogen 11 (2022), 448; Oronsky et al., Transl Oncol 11 (2018), 771-778). Therefore, prevention and treatment of CCRT-induced RIOM is of great clinical importance. Currently, RIOM is best treated through prophylactic measures, and several studies and clinical trials of palliative care medications have attempted to prevent and control RIOM. However, there is a lack of high-level evidence for a single target therapy for the treatment of RIOM.

Radiotherapy induced-toxicity includes xerostomia and mucositis due to the disruption of the mucosal barrier, leading to a drastic oropharyngeal dysbiosis throughout the course of CCRT frequently coincided with the onset of severe mucositis. Probiotic supplements that alter the gut microbiota have been studied to decrease the risk of cancer therapy induced oral mucositis (Shu et al., Oral Oncology 102 (2020), 104559), however, the restoration of oropharyngeal microbiota during CCRT has not been reported. Oropharyngeal probiotic has been shown in previous studies to inhibit the growth of pathogens in the periodontal biofilm and to help maintain the physiological balance of the oral microbiota (Begid et al., Int J Mol Sci 24 (2023), 7249). It also improves the host's immune level, as evidenced by the reduction of anti-inflammatory responses and pro-inflammatory cytokines induced by the strain (Evivie et al., Front Microbiol 10 (2019), 782). Previous clinical studies have shown that oropharyngeal probiotic can effectively colonize the oropharynx/nasopharynx and reduce the risk of pharyngotonsillitis infection (Wilcox et al., Clin Microbiol Infect 25 (2019), 673-680). For these reasons, a pilot study has been designed to investigate the safety and whether the oropharyngeal probiotic could reduce the incidence of RIOM and the delay of RIOM progression in patients with NPC undergoing CCRT.

RIOM has been conceptualized as a result of radiation induced damage to rapidly dividing submucosal basal cells, resulting in epithelial cell damage, which is a common complication arising from nasopharyngeal carcinoma (NPC) patients undergoing concurrent chemoradiation therapy (CCRT).

### Primary Objective

The aim of this study was to assess efficacy and safety of oropharyngeal probiotic ENT-K12 on the incidence, severity, and disease progression of RIOM in NPC patients receiving CCRT, wherein RIOM is scored clinically by the same radiation therapist 5 times a week, according to the Radiation Therapy Oncology Group (RTOG) criteria (see Table 8), to trace dynamic changes of RIOM through the entire course.

TABLE 8
RTOG Criteria

| Grade | Description |
|---|---|
| 0 (none) | No change over baseline |
| I (mild) | Irritation, oral mucosal congestion, erythema, may experience slight |
| | pain, not requiring analgesia |
| II (moderate) | Patchy mucositis that may produce inflammatory serosanguinitis |
| | discharge, lamellar mucositis, inflammatory discharge, edoma or ulcers; |
| | may experience moderate pain requiring analgesia, able to eat fluids |
| III (severe) | Confluent, fibrinous mucositis, edema, ulceration, may include severe |
| | pain requiring narcotic |
| IV (life-threatening) | Ulceration, hemorrhage, bleedings, necrosis, severe pain, inability to eat |

### Secondary Objective

To assess pathogenesis of RIOM (supragingival plaque, saliva, oral microbial dysbiosis, overgrowth of anaerobic bacteria along with an elevated expression of nitrate reductase gene, napA, and regulation of the TLR2/TLR4 signaling pathway in the development of RIOM)
To assess anti-inflammatory effect actions on oral mucosa or tissues (CD3+ T cells, CD4+ T cells and CD8+ T cells)
Nutritional status (Body weight loss, BMI, albumin, prealbumin and NRS2002 score monitored on weekly base)
To assess the quality of life, such as reduction of side effect caused by radiotherapy and the improved quality of life (sore throat, sense of taste and appetite, dry mouth, neck skin peeling, difficult to swallow, hair lose, secretions in the ear, feeling sick or vomit, tiring, diarrhea, etc.)
To confirm safety in subjects, judged by adverse event reporting
Recovery rate
To assess efficacy of oropharyngeal probiotic ENT-K12 on episodes of respiratory tract infections during the study
Days under treatment with antibiotics
Days under treatment with anti-viral drugs
Days under treatment with antipyretics
Days experiencing oral mucositis and tongue ulceration
Days experiencing gingival bleeding or gum diseases

### Synopsis of the Study

A randomized controlled clinical trial was conducted to determine the effect of oropharyngeal probiotic ENT-K12 on preventing the radiotherapy induced oral mucositis (RIOM) among stage II nasopharyngeal carcinoma (NPC) patients. A total of 10 patients, between the ages of 18 and 65 years, which have been diagnosed at least as stage II nasopharyngeal carcinoma (NPC) according to the International Union Against Cancer/American Joint Committee on Cancer TNM classification system, 8th edition for the very first time, who have never experienced head-and-neck radiotherapy were eligible for this study. Patients diagnosed with NPC were randomly assigned (1:1) to receive either the oropharyngeal probiotic (CCRT-P group) or no probiotic intervention (CCRT group) as control group while all patients received CCRT and basic oral hygiene instructions. Patients in the oropharyngeal probiotic group received additional oropharyngeal probiotic intervention 2 weeks prior to and during CCRT. In addition, participants underwent a series of index assessments including blood tests, abdominal ultrasound, chest computed tomography scan, head and neck magnetic resonance imaging scan, and whole-body bone scan. Exclusion criteria for patient enrollment were the following: having an immune disorder, having OM or having recurrent OM prior to CCRT, inability to take OM, previous tumors, and allergy to probiotics. The study was conducted in accordance with the Declaration of Helsinki and with the approval of the ethics committee of Wuhan University of Science and Technology (registration number 2021101). Data were obtained with informed consent from the patients. The investigators evaluated RIOM using The Radiation Therapy Oncology Group (RTOG) guidelines (Table 8), grading from I to IV according to the severity of OM.

### Trial Protocol

All patients underwent CCRT according to National Comprehensive Cancer Network guidelines. Before radiation treatment began, all patients received a full-mouth clinical examination and oral hygiene instruction. Surgical procedures were carried out in patients with caries, pulpal diseases and gingivitis, including professional dental cleaning, filling, endodontic treatment, and extraction of non-restorable teeth. For all recruited patients, radiotherapy was administered 5 times per week at 2 Gy per fraction in a total dose of 60-70 Gy for 7 weeks and the concurrent chemotherapy as listed in Table 9 was applied:

TABLE 9
CCRT dosages

| Patient | CCRT dosage |
|---|---|
| 1 | Chemotherapy: albumin-bound paclitaxel 400 mg d + Nedaplatin 50 mg d2- |
| | 4Q3W; |
| | Immune therapy: Sintilimab 200 mg |
| 2 | Chemotherapy: albumin-bound paclitaxel 260 mg/m2400 mg (200 mg d1, |
| | 200 mgd8) + Nedaplatin 80 mg/m~2140 mg (70 mg d, d2) - Q3W |
| 3 | Chemotherapy: albumin-bound paclitaxel 200 mgd1, 8 + Nedaplatin |
| | 60 mgd1, 65 mg d2; |
| | Targeted therapy: Nimotuzumab 200m, 8 times |
| 4 | Chemotherapy: albumin-bound paclitaxel 200 mgd1, d8 + Nedaplatin |
| | 70 mgd1-2Q3W |
| 5 | Chemotherapy: albumin-bound paclitaxel 260 mg/m²400 mgd1, Nedaplatin |
| | 80 mg/m60 mgd1-2 - Q21D; |
| | Targeted therapy: Toripalimab 240 mg + Nimotuzumab 200 mg d1 - Q7D. |
| 6 | Chemotherapy: albumin-bound paclitaxel 200 mg d1, d2 + Carboplatin |
| | 500 mg 1 |
| 7 | Chemotherapy: albumin-bound paclitaxel 200 mg d1, d2 + Carboplatin |
| | 500 mg 1 |
| 8 | Chemotherapy: albumin-bound paclitaxel 200 mg d1, 8 + Cisplatin |
| | 60 mgd1-2 - Q3W; |
| | Targeted therapy: Nimotuzumab 100 mg - Q7D |
| 9 | Chemotherapy: albumin-bound paclitaxel 200 mg d1, 8 + Cisplatin |
| | 60 mgd1-2 - Q3W |
| 10 | Chemotherapy: albumin-bound paclitaxel 200 mg d1, 8 + Cisplatin |
| | 60 mgd1-2 - Q3W |

Participants have randomly assigned to one of two groups:
Oropharyngeal probiotic ENT-K12 (CCRT-P)
Control (no treatment) (CCRT)

Subjects in the CCRT-P group took a single lozenge 4 times a day, in the form of a slow dissolving lozenge every day, after breakfast, lunch, dinner and brushing their teeth and applying mouthwash before bed, respectively, for a period of 7 weeks during the radiotherapy and 2 weeks prior to the CCRT. The participants were required to suck the lozenge until it is fully dissolved (approximately 4-5 minutes) and to make sure that the lozenge was not chewed or directly swallowed, meanwhile they were suggested not to drink or swallow anything for 2 h following the administration of oropharyngeal probiotic. Uniformly trained doctors and nurses were appointed as clinical symptom observers, who recorded patients' RIOM on a daily basis. The primary objective were the safety of oropharyngeal probiotics and the severity and onset time of RIOM occurrence during CCRT. The study doctors and nurses also recorded the type and severity of adverse events in case oropharyngeal probiotic-related adverse events occurred during the study, the survival, distant metastasis and recurrent NPC were also follow-up for 18 months after CCRT.

### Inclusion Criteria

To be considered eligible for enrolment into the study, subjects must meet the following criteria:
Patients who are diagnosed as stage II nasopharyngeal carcinoma (NPC) for the first time at the time of enrolment
18-65 years of age
Never experiences radiotherapy
Without active symptoms of respiratory tract infections
Without active symptoms of bacteremia
Without active symptoms of organ dysfunction
Being able to tolerant and finish this study
Has signed written informed consent

### Exclusion Criteria

Subjects will be excluded from the study if they meet the following criteria:
Patients who are diagnosed as stage III and IV nasopharyngeal carcinoma (NPC) at the time of enrolment
Patients who had prior head-and-neck radiation therapy
Patients who presented with poor oral hygiene and/or had developed severe forms of periodontal diseases
Active symptoms of respiratory tract infection at the time of recruitment
Active symptoms of bacteremia
Active symptoms of organ dysfunction
With chronic respiratory diseases which may affect the observation
With chronic oral mucositis and ulceration which may affect the observation
   Detailed Study Procedures as outlined in the Clinical Trial Protocol
   Screening Visit & 1^{st} to 35^{th} Radio-Treatment Visits

At the screening visit (Visit 1) the overall details of the study and the procedures to be undertaken will be explained to the study subject once confirming the diagnosis and radiotherapy time schedule. The study subject will then be requested to read the subject information sheet and read and sign the informed consent form, and will receive a signed copy.

In detail, the following information will be recorded and procedures carried out:
Written informed consent
Subject No. and Visit Date
Inclusion and exclusion criteria

If the study subject meets the requirements for recruitment, the following information will be recorded and procedures carried out:
Subject Eligibility
Demographic data
Medication History
Oral Mucositis Grading Scales; WHO
Randomization

The study subject will be randomized into the study and assigned to one of two groups: a) Oropharyngeal probiotic ENT-K12; and b) Control (no treatment)

The study subject in the probiotic group will be provided with a 7 week supply of study product and instructed to take a single lozenge 4 times a day, in the form of a slow dissolving lozenge every day, after breakfast, lunch, dinner and brushing teeth and applying mouthwash before bed, respectively, for a period of 7 weeks during the radiotherapy. The study subject will be required to suck the lozenge until it is fully dissolved (approximately 4-5 minutes) and to make sure that the lozenge is not chewed or directly swallowed. They will be suggested not to drink or swallow anything for min. 2 h following the administration of the probiotics. The study subject will be instructed to return any unused study product at their final visit on the day that a full course of radiotherapy is conducted.

The study subject will be instructed to follow their standard medication, nursing, diet and exercise for the duration of the study.

The study subject will be instructed to contact the study physician at any time during the study if they are showing symptoms of respiratory tract infections, sore throat, fever more than 38° C., headaches, muscle aches, breathlessness, tight chest, cough, stuffy or runny nose, enlarged lymph nodes and/or the appearance of abscesses (pus) or white patches on tonsils; and showing symptoms of oral mucositis, such as dry mouth, saliva thickening, increasing mucus, shiny swollen red gums, soft white patches or pus on the tongue, sore mouth, blood in the mouth or mild burning sensation while eating. If there is evidence of respiratory tract infection and oral mucositis, they will be asked to attend the study site so the diagnosis can be confirmed.

The study subjects will be asked to record the days of experiencing oral mucositis, tongue ulceration, gingival bleeding and gum diseases during the study period of 15 weeks.
Self-Assessment of Overall Health

Please ask the subject about its overall health and indicate the results on the 5-point scale.
Appointment for 1^{st} to 35^{th} radio-treatment visits

The study subject will be provided with an appointment to return to the study site for their 35 visits (5 visits per week for 7 weeks alone with each radiotherapy treatment during the full course of radiotherapy).
Appointment for final visit

The study subject will be provided with an appointment to return to the study site for their final visit on the day that a full course of radiotherapy is conducted.

### Sick Visit (Visit for Diagnosis of Respiratory Tract Infections and Oral Mucositis)

The study subject will be instructed to contact the study physician at any time during the study if they are showing symptoms of respiratory tract infections, oral mucositis and tongue ulceration, such as:
sore throat,
fever more than 38° C.,
headaches,
muscle aches,
breathlessness,
tight chest,
cough,
stuffy or runny nose
enlarged lymph nodes and/or the appearance of abscesses (pus) or white patches on tonsils.
dry mouth,
saliva thickening,
increasing mucus,
shiny swollen red gums,
soft white patches or pus on the tongue,
sore mouth,
blood in the mouth or mild burning sensation while eating

If there is evidence of a respiratory tract infection, the subject will be asked to attend the study site. A throat swab will be collected and rapid swab test to detect the respiratory pathogen will be performed. The physician will advise regarding medications/medical treatment as required.

The study subject will continue taking the study product, in accordance with the instructions provided, throughout the study period. In case antibiotic treatment is required, the study subject will continue taking the study product during the antibiotic course.

The following information will be recorded and procedures carried out during the “Sick Visit”:
Subject No. and Visit Date
Diagnosis (respiratory tract infection, oral mucositis, tongue ulceration) (please indicate the type of respiratory tract infection (e.g., Streptococcal pharyngitis/tonsillitis) and if the infection has been confirmed by rapid throat swab (RAD) or other microbial test)
Medication prescribed (stating the date and duration of antibiotic, anti-viral drug, antipyretics/steroids, anti-fungi courses)
Oral Mucositis Grading Scales; WHO
Days of oral mucositis and tongue ulceration
Days of gingivitis and gum diseases
Progression of upper respiratory tract infection to lower respiratory tract infection (eg, chest CT showing ground-glass pattern)
Requiring hospitalization or ICU due to severe symptoms and hypoxemia
Pulmonary complications (decreased FEV1, oxygen diffusion capacity)
Adverse Events (please asked the study subject about adverse events and—in case adverse events occurred during the treatment—indicate type and severity of adverse events on the separate document “Adverse Events”)
Self-Assessment of Overall Health (please ask the study subject about its overall health and indicate the results on the 5-point scale).
   Final Visit (on the Day that a Full Course of Radiotherapy is Conducted)

The following information will be recorded and procedures carried out during the Final visit:
Subject No. and Visit Date
Medical History over the past 7 weeks (upper respiratory tract infections, progression of URTi to LRTi, requiring hospitalization, pulmonary complications, incidence and etiology of diarrhea) (please indicate the type and number of respiratory tract infections, oral mucositis and tongue ulceration not being diagnosed during the “Sick Visit(s)” and if the infection has been confirmed by rapid throat swab (RAD) or other microbial test)
Oral Mucositis Grading Scales; WHO
Days experiencing oral mucositis and tongue ulceration over the past 7 weeks
Days experiencing gingivitis and gum diseases over the past 7 weeks
Adverse Events
Compliance (return unused study product and assess compliance)
Self-Assessment of Overall Health (please ask the subject about its overall health and indicate the results on the 5-point scale)
Treatment with antibiotics over the past 7 weeks
Treatment with anti-viral drugs over the past 7 weeks
Treatment with antipyretics/steroids over the past 7 weeks
Treatment with anti-fungi drugs over the past 7 weeks
Tolerability and global assessment of treatment effects
End of treatment
Appointment for follow-up visit (8 weeks after final visit)
   Follow-Up Visit (8 Weeks after Final Visit)

The following information will be recorded and procedures carried out during the Follow-up visit:
Subject No. and Visit Date
Medical History during the last 8 weeks (upper respiratory tract infections, progression of URTi to LRTi, requiring hospitalization, pulmonary complications, incidence and etiology of diarrhea) (please indicate the type and number of upper respiratory tract infections suffered during the last 8 weeks and if the infection has been confirmed by rapid throat swab (RAD) or other microbial test)
Treatment with antibiotics over the past 8 weeks
Treatment with anti-viral drugs over the past 8 weeks
Treatment with antipyretics/steroids over the past 8 weeks
Treatment with anti-fungi drugs over the past 8 weeks
Oral Mucositis Grading Scales; WHO
Days experiencing oral mucositis and tongue ulceration over the past 8 weeks
Days experiencing gingivitis and gum diseases over the past 8 weeks
Adverse Events
Self-Assessment of Overall Health (please ask the subject about its overall health and indicate the results on the 5-point scale)
End of study

### Concomitant Medication

The study subject will be questioned about their medication history. The details of any medication taken will be recorded in the case notes and case report form.

### Study Product

Participants in the probiotic group took an oropharyngeal probiotic lozenge, formulated in the form of oral lozenges comprising the below stated composition. Each lozenge contains not less than 1 billion colony forming units (CFUs) of Streptococcus salivarius ENT-K12 (also known as Streptococcus salivarius subsp. thermophilus ENT-K12, DSM 34540) over shelf life.

TABLE 10
Study product

| Active ingredients | Function | Amount | Percentage |
|---|---|---|---|
| Streptococcus | Active ingredient | 50 mg | 5% |
| salivarius subsp. | | | |
| thermophilus | | | |

| Excipients | Function | Amount | Percentage |
|---|---|---|---|
| Crystalline fructose | Bulking agent | 635 mg | 63.5% |
| Maltodextrin | Bulking agent | 290 mg | 29% |
| Magnesium stearate | Anticaking agent, | 15 mg | 1.5% |
| | lubricant | | |
| Strawberry flavor | Flavour | 10 mg | 1% |

### Adverse Event Grading and Assessments

AEs will be graded on a three-point scale and reported in detail as indicated on the CRF:
Mild—easily tolerated, causing minimal discomfort and not interfering with normal everyday activities.
Moderate—sufficiently discomforting to interfere with normal everyday activities.
Severe—incapacitating and/or prevents normal everyday activities.

Relationship assessment: Study treatment relationship for each AE should be determined by the investigator using the following explanations:
Unrelated—the event is clearly related to other factors such as the patient's clinical state, therapeutic interventions, or a concomitant medication administered to the patient and does not follow a known response pattern to the investigational product.
Possible—the event follows a reasonable temporal sequence from the time of investigational product administration and/or follows a known response pattern to the study treatment, but could have been produced by other factors such as the patient's clinical state, therapeutic interventions, or concomitant medications administered to the patient.
Definite—the event follows a reasonable temporal sequence from the time of investigational product administration and follows a known response pattern to the investigational product and cannot be reasonably explained by other factors such as the patients clinical state, therapeutic interventions administered to the patient and either occurs immediately following investigational product, or improves on stopping the investigational product, or reappears on repeat exposure, or there is a positive reaction at the application site.

### Statistically Analysis

Comparison of means of continuous numerical variables that conformed to normal distribution with chi-squared variance was performed using the t test, and continuous numerical variables that did not conform to normal distribution were tested nonparametrically. Data for continuous variables were reported as mean±standard deviation or median and upper and lower quartiles.

Fisher's exact test was used to compare rates between the two groups. Data were analysed and plotted using SPSS 26.0 and GraphPad Prism 9 software. All tests were two-tailed and differences were considered statistically significant if p<0.05.

### Results

A total of 10 patients were selected to participate in this randomized-controlled pilot study. 5 patients received 4 lozenges of oropharyngeal probiotic every day for a duration of 9 weeks, including 2 weeks prior to the CCRT and 7 weeks of CCRT period, and the other 5 patients did not receive oropharyngeal probiotics served as the control group. Treatment compliance was 100% in this trial, and no patient dropped out of this study. As shown in Table 11, there was no difference in basic characteristics between the two groups. The CCRT-P group consisted of 4 male and 1 female whose clinical stages were newly diagnosed as phase II for 2 patients, phase III for 2 patients and phase IV for 1 patient, while the CCRT group consisted of 3 male and 2 female whose clinical stage were all newly diagnosed as phase III. The median total target volume of radiation is 48.4 and 51.8 for CCRT and CCRT-P groups, respectively. The analysis of baseline characteristics showed no significant difference in terms of gender, age, total radiation target volume, tumor stage/node stage/metastasis stage (TNM stage system), and clinical stages between the CCRT and CCRT-P groups (P>0.05). None of patients' treatment was interrupted or delayed due to RIOM in this study.

TABLE 11
Demographics and characteristics of patients eligible for enrollment

| Variable | CCRT Group (n = 5) | | CCRT-P Group (n = 5) | | P |
|---|---|---|---|---|---|
| Total enrollment, No. (%) | 5 | (50) | 5 | (50) | — |
| Male:female, No. (%) | 3 (60):2 (40) | | 4 (80):1 (20) | | 1.0 |
| Age, mean ± SD, y | 49.00 ± 7.00 | | 48.60 ± 15.98 | | 0.96 |
| Total target volume of radiation, | 72.60 | (71.28~72.60) | 70.40 | (70.18~71.50) | |
| Median (upper and lower | | | | | 0.17 |
| quartiles) | | | | | |
| Tumor stage, No. (%) | | | | | 0.29 |
| T1 | 0 | | 0 | | |
| T2 | 1 | (20) | 4 | (80) | |
| T3 | 2 | (40) | 1 | (20) | |
| T4 | 2 | (40) | 0 | | |
| Node stage, No. (%) | | | | | 0.44 |
| N0 | 0 | | 1 | (20) | |
| N1 | 0 | | 1 | (20) | |
| N2 | 5 | (100) | 3 | (60) | |
| N3 | 0 | | 0 | | |
| Metastasis stage, No. (%) | | | | | 1.0 |
| M0 | 5 | (100) | 4 | (80) | |
| M1 | 0 | | 1 | (20) | |
| Clinical stages | | | | | 0.17 |
| Phase II | 0 | | 2 | (40) | |
| Phase III | 5 | (100) | 2 | (40) | |

| | | | |
|---|---|---|---|
| Phase IV | 0 | 1 | (20) |

Initial assessment indicated that oropharyngeal probiotic intervention is safe to NPC patients undergoing radiotherapy, while a decreased incidence and severity of radiation-induced oral mucositis (RIOM) was observed as shown in Tables 12 and 13. During the 7 weeks of radiation therapy, the incidence of RIOM with RTOG ≥2 was reduced. Furthermore, there was a 3-week delay (the 3^{rd} week for control group and 6^{th} week for probiotic group) in the onset of RIOM with RTOG ≥2 for ≥60% patients, while patients taking oropharyngeal probiotic were fully protected from severe RIOM classified as RTOG 3 and 4.

TABLE 12
The severity of RIOM (scored by RTOG) during the 7-weeks study

| | Ctr. | K12 | Ctr. | K12 | Ctr. | K12 | Ctr. | K12 | Ctr. | K12 | Ctr. | K12 | Ctr. | K12 |
|---|---|---|---|---|---|---|---|---|---|---|---|---|---|---|
| | week 1 | | week 2 | | week 3 | | week 4 | | week 5 | | week 6 | | week 7 | |
| RTOG4 | 0% | 0% | 0% | 0% | 0% | 0% | 0% | 0% | 0% | 0% | 20% | 0% | 20% | 0% |
| RTOG3 | 0% | 0% | 0% | 0% | 0% | 0% | 40% | 0% | 60% | 0% | 40% | 0% | 40% | 0% |
| RTOG2 | 0% | 0% | 0% | 0% | 60% | 0% | 60% | 20% | 40% | 20% | 40% | 60% | 40% | 60% |
| RTOG1 | 0% | 0% | 20% | 0% | 20% | 40% | 0% | 40% | 0% | 60% | 0% | 40% | 0% | 40% |
| RTOG0 | 100% | 100% | 80% | 100% | 20% | 60% | 0% | 40% | 0% | 20% | 0% | 0% | 0% | 0% |

TABLE 13
The progression of RIOM (classified by RTOG level ≥2) during the 7-weeks study

| | Ctr. | K12 | Ctr. | K12 | Ctr. | K12 | Ctr. | K12 | Ctr. | K12 | Ctr. | K12 | Ctr. | K12 |
|---|---|---|---|---|---|---|---|---|---|---|---|---|---|---|
| | week 1 | | week 2 | | week 3 | | week 4 | | week 5 | | week 6 | | week 7 | |
| RTOG ≥2 | 0% | 0% | 0% | 0% | 6% | 0% | 100% | 20% | 100% | 20% | 100% | 100% | 20% | 60% |
| RTOG <2 | 100% | 100% | 100% | 100% | 40% | 100% | 00% | 80% | 0% | 80% | 0% | 0% | 40% | 40% |

During a more detailed assessment, it was shown that there were 60% of patients who experienced severe RIOM (RTOG≥3) and those patients were all in the CCRT (control) group, the incidences for RTOG grade 1, 2, 3 and 4 RIOM in the CCRT group and the CCRT-P group were 0%, 40%, 40% and 20%, and 40%, 60%, 0% and 0%, respectively.

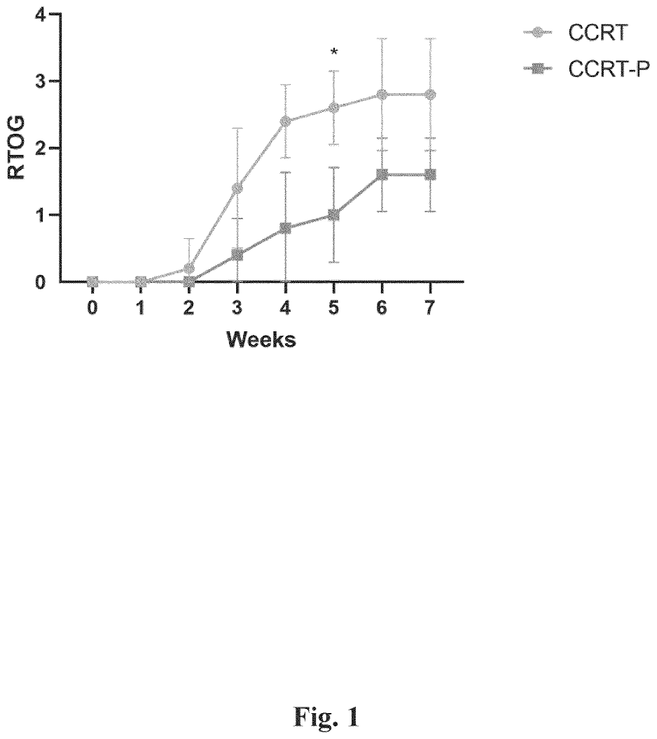

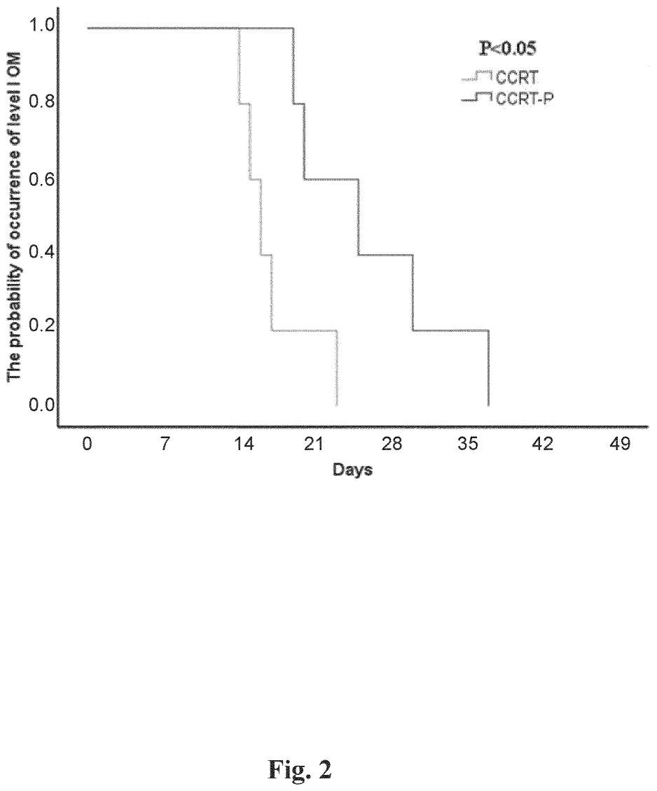

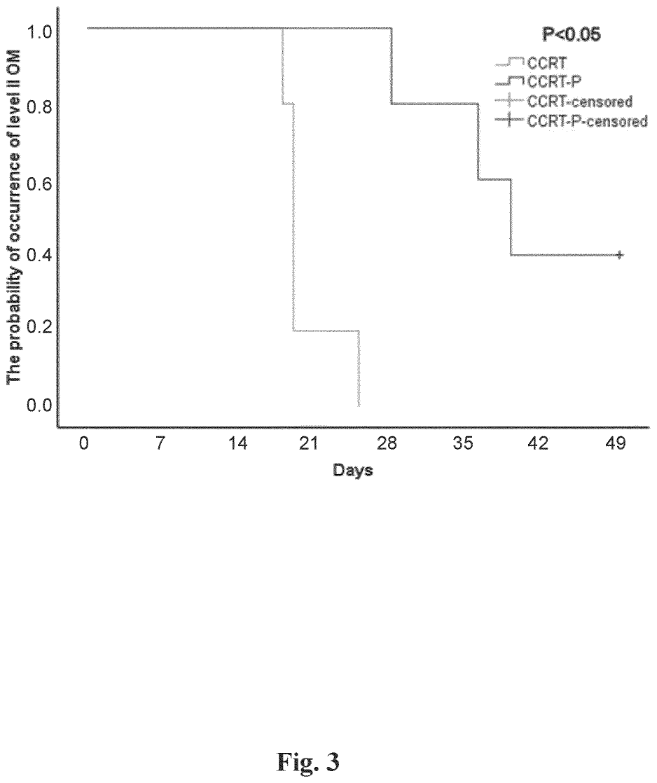

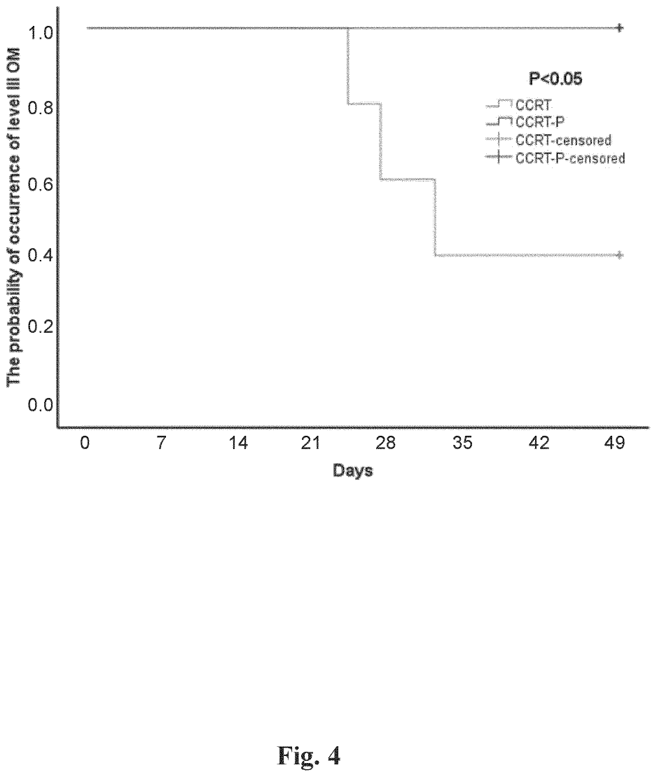

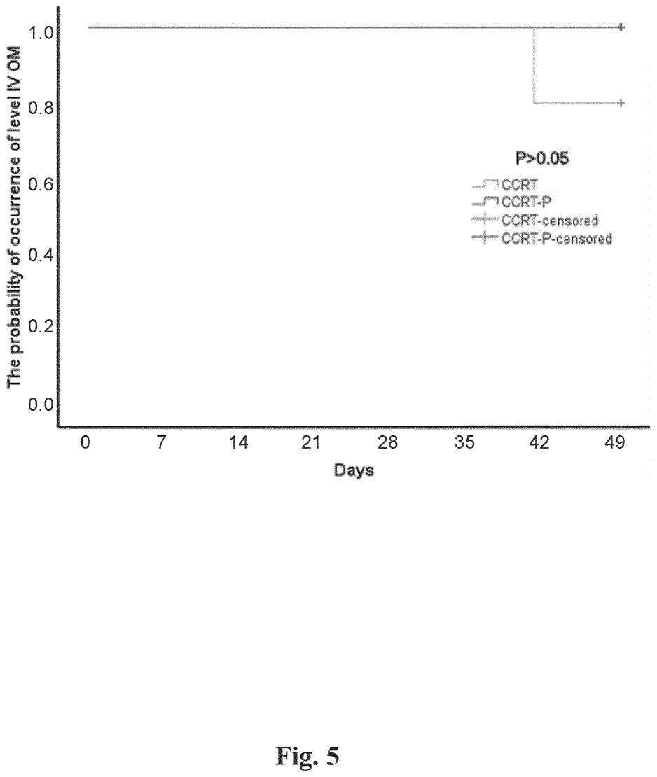

FIG. 1 shows the mean RTOG score that the patients experienced during the 7 weeks of CCRT, wherein the onset of RIOM (RTOG=1) started to occur during the 2^{nd} and 3^{rd} week of the CCRT course for CCRT and CCRT-P groups, respectively. There was a 42.9% reduction in the maximum severity of mean RIOM at 7^{th} week of CCRT in CCRT-P group, where mean RTOG 2.8±0.84 and 1.6±0.55 for CCRT and CCRT-P groups were observed, respectively. The mean interval between the start of CCRT and the maximum severity of RIOM were 5 weeks and 4 weeks for CCRT and CCRT-P groups, respectively. A significant difference in the distribution of RIOM between two groups was observed at the 5^{th} week of the CCRT course (p=0.0309), where mean RTOG 2.6±0.55 and 1.0±0.71 for CCRT and CCRT-P groups were observed, respectively. The detailed mean RTOG scores of patients with different clinical stages during the 7-week CCRT course are shown in Table 14, it can be seen that there was no strong relationship between the mean change of RIOM severity and the clinical stages, but the RIOM severity over time gradually increased alone with the CCRT course. Overall, a trend of reduced RIOM severity by half and delayed onset of RIOM among patients in the CCRT-P group was observed, further, administration of oropharyngeal probiotic significantly protected patients from the onset of severe RIOM (RTOG≥3) during the 7 weeks of CCRT course. The mean interval between the start of CCRT course and onset of different RIOM levels during the 7 weeks of CCRT course is shown in Table 15, where the average time of RIOM onset for RTOG I to IV are 17, 20, 27 and 41 days, respectively, observed in CCRT group, while the average time of RIOM onset for RTOG I to II are 26 and 34 days, respectively, observed in CCRT-P group, while no one experiencing severe RIOM for RTOG≥3 was observed in CCRT-P group. A median of 9 days delay in RIOM onset with RTOG I, and a 14 days delay in RIOM onset with RTOG II, respectively, were observed in CCRT-P group compared with CCRT group. In FIG. 2-5, the Kaplan-Meier curve shows the comparison for the time of first onset of RIOM with RTOG I-IV, respectively, of each patient in both groups. The time from the start of CCRT course to the first onset of RIOM with RTOG level I ranged from 14 days to 23 days, with a median value of 17 days in patients in the CCRT group, while from 19 days to 37 days, with a median value of 26 days in patients in the CCRT-P group who experienced mild pain (FIG. 2). Similarly, the time from the start of CCRT course to the first onset of RIOM with RTOG level II ranged from 18 days to 25 days, with a median value of 20 days in patients in the CCRT group, while from 28 days to 39 days, with a median value of 34 days in 3 of the 5 patients in the CCRT-P group, and the other 2 patients in the CCRT-P group did not experience moderate pain (FIG. 3). The time from the start of CCRT course to the first onset of RIOM with RTOG level III ranged from 24 days to 32 days with a median value of 27 days in 3 of 5 patients of the CCRT group, while none of the patients in the CCRT-P group experienced severe RIOM (FIG. 4). Only 1 patient in CCRT group experienced bleeding and necrosis while none of the patients in the CCRT-P group experienced such pain (FIG. 5).

TABLE 14
Mean RTOG scores of patients during the 7 weeks of CCRT

| | | Week | Week | Week | Week | Week | Week | Week |
|---|---|---|---|---|---|---|---|---|
| | | 1 | 2 | 3 | 4 | 5 | 6 | 7 |
| CCRT | Phase III (n = 5) | 0 | 0.2 | 1.4 | 2.4 | 2.6 | 2.8 | 2.8 |
| (n = 5) | | | | | | | | |
| CCRT-P | Phase II (n = 2) | 0 | 0 | 0 | 1 | 1 | 1.5 | 1.5 |
| (n = 5) | Phase III (n = 2) | 0 | 0 | 0.5 | 0.5 | 1 | 2 | 2 |
| | Phase IV (n = 1) | 0 | 0 | 1 | 1 | 1 | 1 | 1 |

TABLE 15
Mean interval between the start of CCRT course and onset of
different RIOM levels during the 7 weeks of CCRT course

| | | | Level 3 | Level 4 |
|---|---|---|---|---|
| | Level 1 (day) | Level 2 (day) | (day) | (day) |
| CCRT (n = 5) | 17.0 ± 3.54 | 20.0 ± 2.8 | 27.7 ± 4.0 | 41 |
| CCRT-P (n = 5) | 26.2 ± 7.5 | 34.3 ± 5.7 | — | — |

There was no oropharyngeal probiotic-related adverse event reported throughout the study period; all 10 patients survived longer than 18 months after CCRT in this study, stable disease with no distant metastasis nor recurrent NPC was observed in all patients over a follow-up of 18 months, indicating that oropharyngeal probiotic DSM 34540 is safe for NPC patients having no cariogenic effect during CCRT.

RIOM has a detrimental effect on patients' quality of life, physical function, emotional function, role function and malnutritional risks which all impact therapy outcome. Thus, there are demands for an advanced clinical practice enabling healthcare professionals to prioritize the coping approaches to ease the burden of cancer therapy and improve clinical outcomes of their patients. However, RIOM treatment is challenging, and diverse preventive drugs and natural agents have been clinically tested, however, ones that are affordable or without contradictory data were rare, for example, drugs stimulating epithelial cells proliferation might sustain cancer cells growth makes them considerable approaches, leading to substantial incremental cost, health consultation and economic burden on the cancer patients. In the present study, it was demonstrated that supplementation of oropharyngeal probiotics 2 weeks prior to and during the CCRT resulted in a significant advantage to delay the onset of RIOM and greatly reduce the prevalence of severe RIOM among NPC patients undergoing CCRT while the safety was confirmed by no oropharyngeal probiotic-related adverse event reported throughout the 7 weeks of study and over a follow-up of 18 months without distant metastasis nor recurrent NPC.

### Example 2: Oropharyngeal Probiotic ENT-K12 Prevents Oral Mucositis Among Human Subjects Underwent Hematopoietic Stem Cell Transplantation

Autologous hematopoietic stem cell transplantation (auto-HSCT) is the standard of care for multiple myeloma patients eligible for high-dose therapy, lymphoma patients undergoing second-line treatments and for acute myelogenous leukemia. Immune system impairment and chemotherapies prior to HSCT significantly increase the risk of infections, particularly early onset respiratory tract infections, within 100 days of the transplant (Scarlata et al., Eur Respir J. 49 (2017), 1601902. Thus, respiratory viral infection is common in patients who have undergone (HSCT), which usually presents as an upper respiratory tract infection in this patient population but may progress rapidly to lower respiratory that is associated with mortality rate.

Approximately 10% of auto-HSCT patients experience signs or symptoms of common respiratory viral infections after hospitalization, common respiratory viral pathogens include influenza viruses, parainfluenza virus, adenovirus, human rhinovirus and human coronavirus, and of the respiratory viral infections, approximately 45% were complicated by lower respiratory tract infection while 7% resulted in intensive care admission (Moret et al., Infect Dis (Lond). 53 (2021), 274-280; Marinelli et al., Biol Blood Marrow Transplant. 26 (2020), 782-788. Disease progression of respiratory virus infection along with high mortality in hematologic stem cell transplant recipients and patients with hematologic malignancies are increasingly recognized as a cause of significant morbidity and mortality (Fontana and Strasfeld, Infect Dis Clin North Am. 33 (2019), 523-544. Prophylaxis of respiratory tract infections among HSCT patients are of high importance, specific drugs have been reported as successful treatment after diagnostic molecular tests and multiplex assays, but nasal respiratory swabs are not routinely performed post-transplant after hospitalization, antibiotic prescription according to clinical experiences of practitioners is very common. In regards to reconstruct the dysbiosis of intestinal microflora and immunomodulatory effects after HSCT, numerous studies have demonstrated the outcome of gut probiotics and prebiotic administration as safe and well-tolerated treatment among adult and pediatric auto-HSCT and allo-HSCT patients, which potentially provide benefit on reducing gastrointestinal toxicity or incidence of graft-versus-host-disease (GVHD); see for example Mizutani et al., Intern Med. 62 (2023), 2949-2958; Andermann et al., Transplant Cell Ther. 27 (2021), 932.e1-932.e11; Ladas et al., Bone Marrow Transplant. 51 (2016), 262-266; Gorshein et al., Clin Transplant. 31 (2017), 10.1111/ctr.12947; Yazdandoust et al., Transpl Immunol. 78 (2023), 101836. In an attempt to discover other approaches to reduce the risks of RTi and the progression from upper to lower respiratory tract infections among HSCT patients, this pilot randomized clinical trial was conducted to investigate whether oropharyngeal probiotics are associated with the prevention or reduction in occurrence and severity of respiratory tract infections, hence mitigate antibiotic use following auto-HSCT.

Furthermore, oral mucositis is a common debilitating dose-limiting toxicity of HSCT. The oral microbiome changed significantly after HSCT and returned to origin composition after three months which is a microbially-driven risk factor for oral mucositis. In particular, changes in microbial diversity and similarity were more pronounced and rapid in patients who developed ulcerative oral mucositis. It was reported that Streptococcus species were identified before HSCT, but gradually decreased and were replaced by coagulase-negative Staphylococci, an increase in Candida species after HSCT and the identification of Enterococcus species were significantly associated with ulcerative oral mucositis after HSCT, while patients who did not develop ulcerative oral mucositis had a more resilient microbial ecosystem.

The study product contains the oropharyngeal probiotic strain Streptococcus salivarius subsp. thermophilus ENT-K12 (DSM 34540)—a probiotic strain for oral cavity and upper respiratory tract, associated with ear, nose, and throat health. Previous clinical studies have established the safety and efficacy of daily administration of Streptococcus salivarius subsp. thermophilus ENT-K12 in reducing respiratory tract infections and respiratory key pathogens. The rationale of this study is to establish safety and efficacy of oropharyngeal probiotic ENT-K12 administration in patients underwent Hematopoietic Stem Cell Transplantation (HSCT). The primary purpose of this proposed study is to evaluate the effectiveness of oropharyngeal probiotic ENT-K12 in reducing respiratory tract infections and oral mucositis in patients underwent Hematopoietic Stem Cell Transplantation.

TABLE 16
Oral mucositis grading scale according
to the WHO (World Health Organization)

| Grade | Description |
|---|---|
| 0 (none) | None |
| I (mild) | Oral soreness, erythema |
| II (moderate) | Oral erythema, ulcers, solid diet tolerated |
| III (severe) | Oral ulcers, liquid diet only |
| IV (life-threatening) | Oral alimentation impossible |

### Primary Objective

A multicenter, open-labeled, randomized controlled pilot study was conducted to assess efficacy of oropharyngeal probiotic ENT-K12 on episodes of respiratory tract infections and oral mucositis during 100 days after Hematopoietic Stem Cell Transplantation (HSCT).

### Secondary Objective

To assess efficacy of oropharyngeal probiotic ENT-K12 on reducing episodes of viral respiratory tract infections during 100 days after HSCT
To assess efficacy of oropharyngeal probiotic ENT-K12 on reducing episodes of bacterial respiratory tract infections
To assess efficacy of oropharyngeal probiotic ENT-K12 on reducing episodes of fungal respiratory tract infections
To assess efficacy of oropharyngeal probiotic ENT-K12 on reducing severity of oral mucositis and xerostomia (according to oral mucositis grading scales; WHO)
To assess influence of oropharyngeal probiotic ENT-K12 on recovering of T-lymphocyte subsets
To assess influence of oropharyngeal probiotic ENT-K12 on immune modulation via salivary cytokine
Days under treatment with antibiotics
Days under treatment with anti-viral drugs (ribavirin/pallvizumab)
Days under treatment with antipyretics/steroids
Days under treatment with anti-fungi drugs
Days experiencing oral mucositis
Days experiencing gingivitis
Progression of URTi to LRTi (Chest CT showing ground-glass pattern)
Requiring hospitalization due to severe symptoms and hypoxemia (ICU)
Pulmonary complications (decreased FEV1, oxygen diffusion capacity)
Incidence and etiology of diarrhea
Incidence and grade of acute Graft vs Host Disease (GvHD)
Mortality rate
To confirm safety in subjects, judged by adverse event reporting

### Synopsis of the Study

A study to determine the effect of oropharyngeal probiotic ENT-K12 on preventing the respiratory tract infections and oral mucositis among patients underwent Hematopoietic Stem Cell Transplantation (HSCT).

Up to 60 patients who are diagnosed as a HSCT receiver according to 2016 WHO, between the ages of 18 and 65 years old, of whom the Eastern Cooperative Oncology Group (ECOG) performance status is between 0-2, and hematopoietic system is well reconstructed after HSCT with no active symptoms of lower respiratory infections, chronic respiratory diseases or organ dysfunction will be eligible for this study. At the end, 16 patients meeting these criteria participated in the study. Participants were randomly assigned to one of two treatment groups:
Oropharyngeal probiotic ENT-K12
Control (no treatment)

Subjects in the probiotic group took a single probiotic lozenge 3 times a day, in the form of a slow dissolving lozenge, after breakfast, lunch and brushing their teeth and applying mouthwash before bed, respectively, for a period of 100 days after their hematopoietic system is well reconstructed which is defined as blood platelets >20*10⁹ cells/L and neutrophil >0.5*10⁹ cells/L after HSCT. The participants were needed to suck the lozenge until it was fully dissolved (approximately 4-5 minutes) and to ensure that the lozenge was not chewed or directly swallowed; meanwhile, they were suggested not to drink or swallow anything for 2 hours following the administration of oropharyngeal probiotics.

Participant diagnosis and conditioning regimen was abstracted from the medical record from the initiation of the conditioning regimen until day 130, including the 100 days of intervention and a follow-up of 30 days. Each episode of respiratory tract infections was recorded on a weekly base, if subjects develop symptoms of respiratory tract infection during the study, such as sore throat, fever more than 38° C., headache, muscle soreness, dyspnea, chest tightness, cough, stuffy or runny nose, enlarged lymph nodes, abscesses or white plaques on tonsils, the subject will be instructed to contact the research doctor. During each consultation, any adverse events of the subjects were recorded. Subjects will return unused probiotics at the end of the study, experimental compliance will be assessed by calculating unused lozenges at the end of the consultation; compliance criteria are determined as if ≥90% of the dispensed lozenges have been used.

### Study Population

Patients who are diagnosed as a HSCT receiver according to 2016 WHO, between the ages of 18 and 65 years old, of whom the Eastern Cooperative Oncology Group (ECOG) performance status is between 0-2, and hematopoietic system is well reconstructed after HSCT with no active symptoms of lower respiratory infections, chronic respiratory diseases or organ dysfunction will be eligible for this study. Accordingly, a total of 16 patients who met the above listed criteria were recruited for this study and randomly assigned to probiotic group or control group.

### Inclusion Criteria

To be considered eligible for enrolment into the study, subjects must meet the following criteria:
Patients who are diagnosed as a HSCT receiver according to 2016 WHO
18-65 years of age
Eastern Cooperative Oncology Group (ECOG) performance status 0-2
Without active symptoms of respiratory tract infections
Without active symptoms of bacteremia
Without active symptoms of organ dysfunction
Being able to tolerant and finish this study
Has signed written informed consent

### Exclusion Criteria

Subjects will be excluded from the study if they meet the following criteria:
Eastern Cooperative Oncology Group (ECOG) performance status ≥3
Failure of hematopoietic reconstruction which is defined as blood platelets <20*10⁹ cells/L and neutrophil <0.5*10⁹ cells/L after HSCT
Active symptoms of respiratory tract infection at the time of recruitment
Active symptoms of bacteremia
Active symptoms of organ dysfunction
With chronic respiratory diseases which may affect the observation

### Study Procedures

At the screening visit the overall details of the study and the procedures to be undertaken will be explained to the study subject, once confirming the hematopoietic reconstruction judged by blood platelets ≥20*10⁹ cells/L and neutrophil ≥0.5*10⁹ cells/L after HSCT. The study subject will then be requested to read the subject information sheet and read and sign the informed consent form, and will receive a signed copy.

In detail, the following information will be recorded and procedures carried out:
Written informed consent
Subject No. and Visit Date
Inclusion and exclusion criteria

If the study subject meets the requirements for recruitment, the following information will be recorded and procedures carried out:
Subject Eligibility
Demographic data
Medication History
Oral Mucositis Grading Scales; WHO
Randomization

The study subject will be randomized into the study and assigned to one of two treatment groups: a) Oropharyngeal probiotic ENT-K12; and b) Control (no treatment).

The study subject will be provided with a 100 days' supply of study product and instructed to take 3 single lozenges every day after breakfast, lunch and before bed after brushing their teeth and applying mouthwash, respectively. The study subject will be required to suck the lozenge until it is fully dissolved (approximately 4-5 minutes) and to make sure that the lozenge is not chewed or directly swallowed. They will be suggested not to drink or swallow any substance for min. 2 h following the administration of probiotic. The study subject will be instructed to return any unused study product at their final visit.

The study subject will be instructed to follow their standard medication, nursing, diet and exercise routine for the duration of the study. The study subject will be instructed to contact the study physician at any time during the study if they are showing symptoms of respiratory tract infection, such as sore throat, fever more than 38° C., headaches, muscle aches, breathlessness, tight chest, cough, stuffy or runny nose, enlarged lymph nodes and/or the appearance of abscesses (pus) or white patches on tonsils; and showing symptoms of oral mucositis, such as dry mouth, saliva thickening, increasing mucus, shiny swollen red gums, soft white patches or pus on the tongue, sore mouth, blood in the mouth or mild burning sensation while eating. If there is evidence of respiratory tract infection and oral mucositis, they will be asked to attend the study site so the diagnosis can be confirmed.
Blood sampling for detection of T-lymphocyte subsets (according to standard medical procedure)
Saliva sampling for detection of immune modulation (0,1,3,7,100,130)
Throat and oral mucus swab sampling for detection of key respiratory pathogens (including C. albicans) and reestablishment of oral microflora)
Self-Assessment of Overall Health (please ask the subject about its overall health and indicate the results on the 5-point scale)
Appointment for final visit
The study subject will be provided with an appointment to return to the study site for their final visit (Day 100). The subjects will be asked to record the days they experience symptoms of respiratory tract infections, oral mucositis and gingivitis over the past 100 days (medical staff or medical school student will make weekly telephone calls to monitor their conditions as well)

### Sick Visit (Visit for Diagnosis of Respiratory Tract Infections and Oral Mucositis)

The study subject will be instructed to contact the study physician at any time during the study if they are showing symptoms of respiratory tract infection and oral mucositis, such as:
sore throat,
fever more than 38° C.,
headaches,
muscle aches,
breathlessness,
tight chest,
cough,
stuffy or runny nose
enlarged lymph nodes and/or the appearance of abscesses or white patches on tonsils.
dry mouth,
saliva thickening,
increasing mucus,
shiny swollen red gums,
soft white patches or pus on the tongue,
sore mouth,
blood in the mouth or mild burning sensation while eating

If there is evidence of a respiratory tract infection, the subjects will be asked to attend the study site. A throat swab will be collected and rapid swab test to detect the respiratory pathogen will be performed. The physician will advise regarding medications/medical treatment as required.

The study subject will continue taking the study product, in accordance with the instructions provided, throughout the study period. In case antibiotic treatment is required, the study subject will continue taking the study product during the antibiotic course.

The following information will be recorded and procedures carried out during the “Sick Visit”:
Subject No. and Visit Date
Diagnosis (respiratory tract infection, oral mucositis, diarrhea, acute GvHD) (please indicate the type of respiratory tract infection (e.g., Streptococcal pharyngitis/tonsillitis, respiratory syncytial virus infection) and if the infection has been confirmed by rapid throat swab (RAD) or other microbial test)
Medication prescribed (stating antibiotic, anti-viral drug, antipyretics/steroids, anti-fungi drug)
Oral Mucositis Grading Scales; WHO
Days of oral mucositis
Days of gingivitis
Progression of upper respiratory tract infection to lower respiratory tract infection (eg, chest CT showing ground-glass pattern)
Requiring hospitalization or ICU due to severe symptoms and hypoxemia
Pulmonary complications (decreased FEV1, oxygen diffusion capacity)
Adverse Events (please asked the study subject about adverse events and—in case adverse events occurred during the treatment—indicate type and severity of adverse events on the separate document “Adverse Events”)
Self-Assessment of Overall Health (please ask the study subject about its overall health and indicate the results on the 5-point scale).
   Follow-Up Visit (30 Days after Final Visit) The following information will be recorded and procedures carried out during the Follow-up visit:
Subject No. and Visit Date
Medical History during the last 30 days (upper respiratory tract infections, progression of URTi to LRTi, requiring hospitalization, pulmonary complications, incidence and etiology of diarrhea, incidence and grade of acute GvHD)
Oral Mucositis Grading Scales; WHO
Days experiencing oral mucositis over the past 30 days
Days experiencing gingivitis over the past 30 days (please indicate the type and number of respiratory tract infections and oral mucositis suffered during the last 30 days and if the infection has been confirmed by rapid throat swab (RAD) or other microbial test)
Adverse Events
Self-Assessment of Overall Health (please ask the subject about its overall health and indicate the results on the 5-point scale)
Treatment with antibiotics over the past 30 days
Treatment with anti-viral drugs (ribavirin/pallvizumab) over the past 30 days
Treatment with antipyretics/steroids over the past 30 days
Treatment with anti-fungi drugs over the past 30 days
End of study
   Removal of Subjects from the Study

Subjects will be withdrawn from the study if,
The study subject elects independently to withdraw from the study;
If the study subject develops any condition which contravenes the original criteria;
If the study subject is considered at any point to be unsuitable to continue the study, at the discretion of the investigator.

### Concomitant Medication

The study subject will be questioned about their medication history. The details of any medication taken will be recorded in the case notes and case report form.

### Study Product

Participants in the probiotic group will take an oropharyngeal probiotic lozenge, formulated in the form of oral lozenges having the below mentioned composition. Each lozenge contains not less than 1 billion colony forming units (CFUs) of Streptococcus salivarius ENT-K12 (also known as Streptococcus salivarius subsp. thermophilus ENT-K12) over shelf life.

TABLE 17
Study product

| Active ingredients | Function | Amount | Percentage |
|---|---|---|---|
| Streptococcus | Active ingredient | 50 mg | 5% |
| salivarius subs. | | | |
| thermophilus | | | |

| Excipients | Function | Amount | Percentage |
|---|---|---|---|
| Crystalline fructose | Bulking agent | 635 mg | 63.5% |
| Maltodextrin | Bulking agent | 290 mg | 29% |
| Magnesium stearate | Anticaking agent, | 15 mg | 1.5% |
| | lubricant | | |
| Strawberry flavor | Flavour | 10 mg | 1% |

### Adverse Event Grading and Assessments

AEs will be graded on a three-point scale and reported in detail as indicated on the CRF:
Mild—easily tolerated, causing minimal discomfort and not interfering with normal everyday activities.
Moderate—sufficiently discomforting to interfere with normal everyday activities.
Severe—incapacitating and/or prevents normal everyday activities.

Relationship assessment: Study treatment relationship for each AE should be determined by the investigator using the following explanations:
Unrelated—the event is clearly related to other factors such as the patient's clinical state, therapeutic interventions, or a concomitant medication administered to the patient and does not follow a known response pattern to the investigational product.
Possible—the event follows a reasonable temporal sequence from the time of investigational product administration and/or follows a known response pattern to the study treatment, but could have been produced by other factors such as the patient's clinical state, therapeutic interventions, or concomitant medications administered to the patient.
Definite—the event follows a reasonable temporal sequence from the time of investigational product administration and follows a known response pattern to the investigational product and cannot be reasonably explained by other factors such as the patients clinical state, therapeutic interventions administered to the patient and either occurs immediately following investigational product, or improves on stopping the investigational product, or reappears on repeat exposure, or there is a positive reaction at the application site.

### Statistical Analysis

Comparison of means of continuous numerical variables that conformed to a normal distribution with homoscedasticity was performed using the t test, and continuous numerical variables that did not conform to a normal distribution were tested nonparametrically. Data for continuous variables were reported as the mean±standard deviation or median and upper and lower quartiles. chi-square test was used to compare rates between the two groups. Data were analyzed and plotted using SPSS 26.0 and GraphPad Prism 9 software. All tests were two-tailed, and differences were considered statistically significant if P<0.05.

### Results

Sixteen patients (six women and ten men with a mean age of 39.8 years (range 28~53 years)) with hematopoietic tumor underwent auto-HSCT were enrolled in this study and seven were randomized to receive oropharyngeal probiotics, whereas the other nine served as control taking no probiotics. Treatment compliance was good in this trial, and no patient dropped out of this study. The patient characteristics at randomization are summarized in Table 18.

TABLE 18
Demographic characteristics of auto-HSCT recipients when been enrolled

| | Treatment | Control | |
|---|---|---|---|
| Characteristic | (n = 7) | (n = 9) | P value |
| Gender | | | 0.633 |
| Male | 5 (71.4%) | 5 (55.6%) | |
| Female | 2 (28.6%) | 4 (44.4%) | |
| Patient Age | 40.86 ± 8.59 | 39.11 ± 11.95 | 0.918 |
| BMI | 23.058 ± 1.28 | 22.534 ± 3.09 | 0.874 |
| Diagnose | | | 0.362 |
| non-hodgkin lymphoma | 7 (100%) | 8 (88.9%) | |
| hodgkin lymphoma | 0 (0%) | 1 (11.1%) | |
| Treatment scheme | | | 0.235 |
| BEAM | 2 (28.6%) | 5 (55.6%) | |
| BUCYE | 4 (57.1%) | 1 (11.1%) | |
| BUCY | 1 (14.3%) | 3 (33.3%) | |
| CD34⁺ × 10⁶/Kg | 4.04 ± 2.53 | 5.66 ± 4.962 | 0.874 |
| Febrile Neutropenia positivity | 6 (85.7%) | 6 (66.7%) | 0.762 |
| Serum albumin levels | 35.94 ± 3.85 | 37.46 ± 4.02 | 0.56 |
| Hospitalization Days | 35.86 ± 11.13 | 38.44 ± 6.064 | 0.200 |
| Blood culture positivity | 1 (16.7%) | 0 (0%) | 0.299 |
| Body surface area | 1.67 ± 0.071 | 1.69 ± 0.285 | 0.668 |
| Weight change during hospitalization | −1.71 ± 1.496 | −3.07 ± 1.747 | 0.199 |
| C-reactive protein (CRP) | 10.30 ± 7.421 | 21.54 ± 12.791 | 0.085 |
| Duration of neutropenia | 7.33 ± 0.816 | 8.71 ± 1.604 | 0.094 |
| Duration of thrombocytopenia | 16 ± 13.770 | 16 ± 8.266 | 0.666 |
| Duration of febrile symptoms | 4 ± 1.414 | 4.43 ± 4.036 | 0.829 |
| Days of prophylactic antibiotic use | 6.5 ± 3.674 | 6.43 ± 2.440 | 0.771 |
| Presentation of sore throat | 2 (33.3%) | 3 (42.9%) | 0.725 |
| Presentation of xerostomia | 5 (83.3%) | 4 (57.1%) | 0.308 |

The most common baseline disease was non-hodgkin lymphoma (100% in probiotic group and 88.9% in control group) and the rest was hodgkin lymphoma. Of the 7 patients in probiotic group, 2, 4 and 1 were given with BEAM, BuCyE and BUCY conditioning regimen prior to auto-HSCT, respectively, while 5, 1 and 3 in the control group were given with BEAM, BuCyE and BUCY conditioning regimen prior to auto-HSCT, respectively. Their age, gender distribution, BMI, status of disease at auto-HSCT, median number of infused CD34⁺ cells/kg, serum albumin levels, hospitalization days, weight loss during hospitalization, duration of thrombocytopenia, duration of febrile symptoms, days of prophylactic antibiotic use prior to HSCT were similar between the two groups. The rates of febrile neutropenia, positive symptoms of sore throat and xerostomia were similar between the groups. Patients in control group had a higher average c-reactive protein level and duration of neutropenia when been enrolled but they were not statistically significant. Bloodstream infection (BSI) was documented in one patient of the probiotic group when been enrolled but there was no significant difference of the BSI rate between two groups.

Results Concerning the Effect of the Oropharyngeal Probiotic S. salivarius ENT-K12 on Oral Mucositis

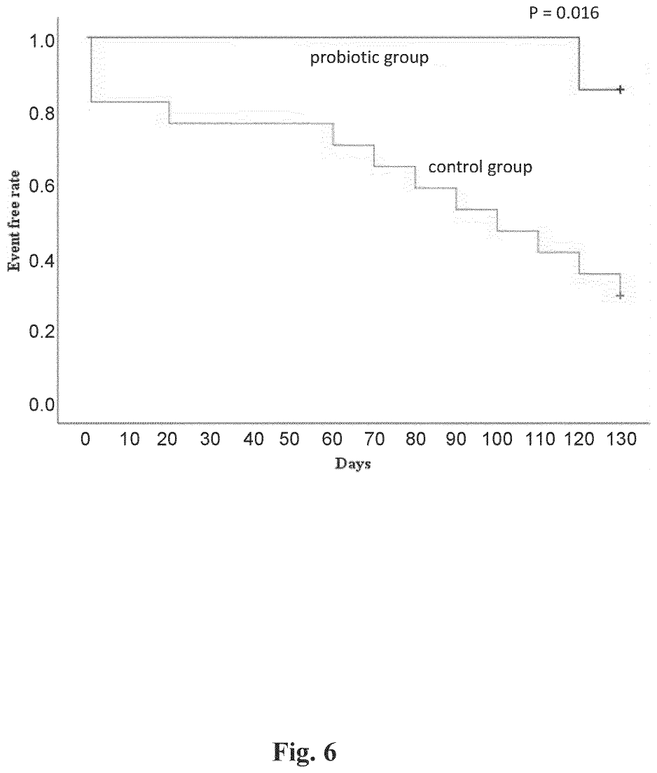

Episodes of ulcerative oral mucositis was monitored three times monthly during the 100 days after reconstructing of hematopoietic system, and followed up for 30 days. Initial assessment indicated that oropharyngeal probiotic intervention is safe to HSCT receiver patients after their hematopoietic system is well reconstructed after HSCT (no adverse effects were observed), while a decreased incidence of ulcerative oral mucositis was observed during the whole study period of 130 days. In particular, the incidence of oral mucositis grade ≥2 according to the WHO grading scale was reduced in patients who received the oropharyngeal probiotic, i.e., the event free rate was higher in patients who received the oropharyngeal probiotic in comparison to the control group. No ulcerative oral mucositis was observed in patients in probiotic group during the 100 days of oropharyngeal probiotic intervention; see FIG. 6.

Concerning that HSCT patients are rendered immunosuppressed, additional caution and comprehensively safety evaluation are needed before the application of probiotics, patients only started the probiotic administration after well reconstructed of their hematopoietic system in this study. Sepsis is one of the worst consequences for patients with mucositis caused by cancer therapy, which may lead to treatment interruption and even life-threating. In this pilot study, no patients developed sepsis due to probiotic administration, and no side effect related with probiotic administration were reported during the whole study period. Besides, patients taking oropharyngeal probiotic were fully protected from ulcerative oral mucositis.

Results Concerning the Effect of the Oropharyngeal Probiotic S. salivarius ENT-K12 on Respiratory Tract Infections

Next to the evaluation of the clinical trial study results as regards oral mucositis, the efficacy and safety of oropharyngeal probiotic on respiratory infection-like symptoms among patients with lymphoma after autologous hematopoietic stem cell transplantation was analyzed.

A trend of more than half decreased respiratory tract infection (RTi) incidence was observed among patients administrating oropharyngeal probiotics during 100 days post-HSCT (29% vs 67%; p=0.131). The incidence of upper respiratory tract infection (URTi) progressing to lower respiratory tract infection (LRTi) was 0% and 16.7% in the probiotic and control group, respectively. Similarly, a trend of approximately two-third reduced RTi incidence rate was observed in probiotic group during the 100 days post-HSCT (0.43% vs 1.00%; p=0.094). Regarding to the RTi-like symptoms, the duration of cough, runny nose and sore throat in probiotic group were shorter than that in control group 100 days post-HSCT (3, 1, 0 days vs 17, 15, 4 days; p=0.090, 0.059, 0.102, respectively), In addition, more than 10 times of reduced days of medication was observed in the probiotic group compared to that in control group during the 100 days post-HSCT, especially a significant statistic difference was observed in days of antibiotic use (0 days of antiviral agent, 1 days of antifungal agent, 0 days of antibiotics vs 1 days of antiviral agent, 0 days of antifungal agent, 15 days of antibiotics; p=0.378, 0.257, 0.024, respectively), while the average days of medication were similar in both groups during the 30 days follow-up (only 0 days vs 0.7 days of antibiotics consumption, respectively; p=0.170). The average duration of each RTi episode in the probiotic group was twice shorter compared to that in control group during the 100 days post-HSCT. Under the circumstances of less antibiotic consumption mentioned above, however, the average duration of each RTi episode were very similar in both groups (1.4 days vs 0.7 days; p=0.816). A statistic difference was observed in the average days of antibiotic use during the whole study period of 130 days, the days of antibiotic use per person in probiotic group was significantly lower than that in control group (0 day vs 15 days; p=0.011), as shown in Table 21.

TABLE 19
Respiratory tract infection-related clinical outcomes during
the 100 days of oropharyngeal probiotic administration

| | Treatment | Control | |
|---|---|---|---|
| | (n = 7) | (n= 9) | P value |
| Incidence of RTi(N/n, %) | 28.57 | 66.67 | 0.131** |
| Incidence of URTi progressing to LRTi (%) | 0 | 16.67 | 0.362** |
| Incidence rate of RTi (times/person/day, %) | 0.43 | 1.00 | 0.094** |
| Presentation of RTi symptoms(days/person) | | | |
| Cough | 3.29 ± 6.18 | 17.22 ± 24.77 | 0.090* |
| Runny nose | 1 ± 2.64 | 14.56 ± 25.44 | 0.059* |
| Sore throat | 0 | 3.78 ± 5.78 | 0.102* |
| Duration of RTi episode (days/episode) | 6.25 ± 10.67 | 13.92 ± 24.42 | 0.243* |
| Days of medication | | | |
| Antiviral agent (days/ person) | 0 | 1.11 ± 3.333 | 0.378* |
| Antifungal agent (days/ person) | 1 ± 2.646 | 0 | 0.257* |
| Antibiotic (days/ person) | 0 | 14.56 ± 25.866 | 0.024* |

*Mann-Whitney U test,
**Chi-Squared Test

TABLE 20
Respiratory tract infection-related clinical outcomes
during the 30 days of follow-up period

| | Treatment | Control | |
|---|---|---|---|
| | (n = 7) | (n = 8) | P value |
| Incidence of RTi(N/n, %) | 14.29 | 25 | 0.605** |
| Incidence rate of RTi (times/person/day, %) | 0.48 | 0.83 | 0.687** |
| Presentation of RTi symptoms(days/person) | | | |
| Cough | 2.86 ± 7.55 | 0.75 ± 1.38 | 0.740* |
| Runny nose | 0 | 0.75 ± 1.38 | 0.170* |
| Sore throat | 2.86 ± 7.55 | 0 | 0.285* |
| Duration of RTi episode (days/episode) | 1.43 ± 3.78 | 0.67 ± 1.32 | 0.816* |
| Days of medication | | | |
| Antiviral agent (days/ person) | 0 | 0 | |
| Antifungal agent (days/ person) | 0 | 0 | |
| Antibiotic (days/ person) | 0 | 0.75 ± 1.38 | 0.170* |

*Mann-Whitney U test,
**Chi-Squared Test

TABLE 21
Respiratory tract infection-related clinical outcomes
during the 130 days of whole study period

| | Treatment | Control | |
|---|---|---|---|
| | (n = 7) | (n = 9) | P value |
| Incidence of RTi episode (N/n, %) | 28.57 | 66.67 | 0.131** |
| Incidence of URTi progressing to LRTi (%) | 0 | 16.67 | 0.362** |
| Incidence rate of RTi (times/person/day, %) | 0.44 | 0.94 | 0.094** |
| Presentation of RTi symptoms (days/person) | | | |
| Cough | 6.14 ± 10.96 | 17.89 ± 24.70 | 0.257* |
| Runny nose | 1 ± 2.64 | 15.22 ± 25.44 | 0.059* |
| Sore throat | 2.86 ± 7.55 | 3.78 ± 5.78 | 0.531* |
| Duration of RTi episode (days/episode) | 6.67 ± 10.00 | 12.36 ± 22.81 | 0.363* |
| Days of medication | | | |
| Antiviral agent (days/ person) | 0 | 1.11 ± 3.333 | 0.378* |
| Antifungal agent (days/ person) | 1 ± 2.646 | 0 | 0.257* |
| Antibiotic (days/ person) | 0 | 15.22 ± 25.548 | 0.011* |

*Mann-Whitney U test,
**Chi-Squared Test

The patient with bacteremia who got a positive result in blood culture test when been enrolled in the probiotic group didn't develop sepsis during the whole study period. Sepsis combining pneumonia post-HSCT occurred in 1 of the 9 patients in the control group.

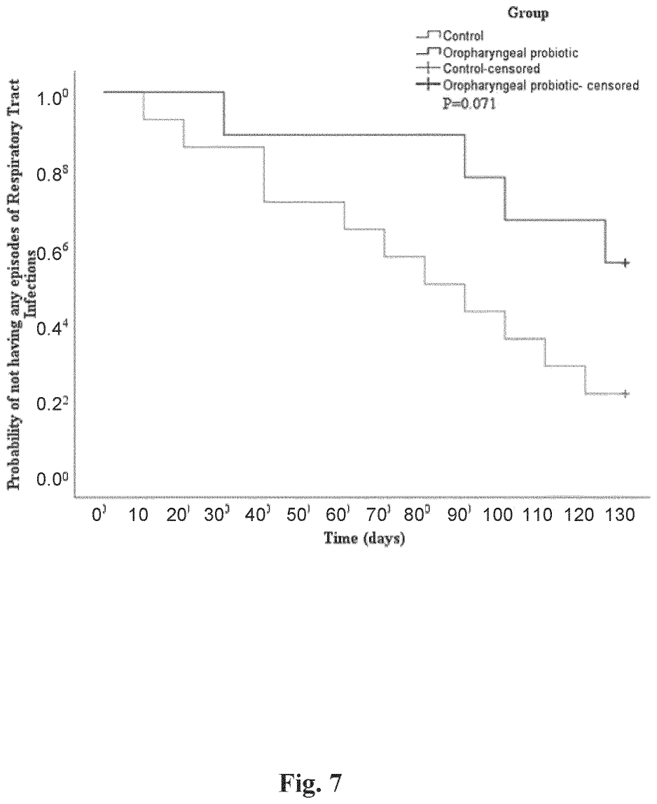

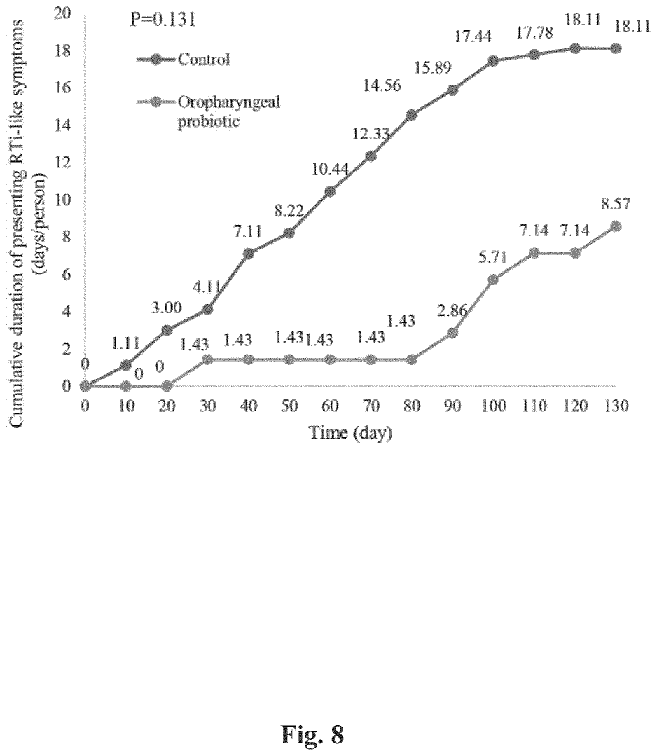

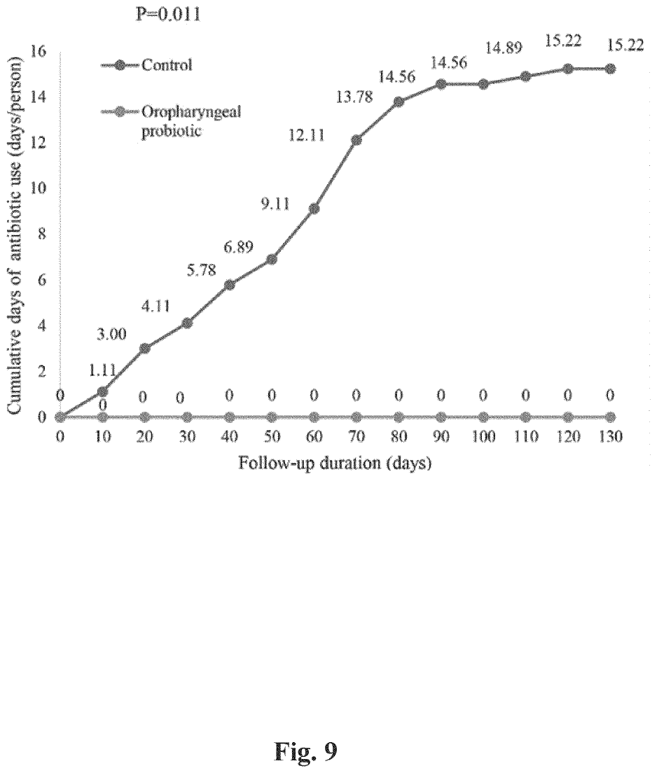

The Kaplan-Meier analysis indicated that a constantly higher probability of not having RTi episode was observed in patients of probiotic group than that in the control group (p=0.071; FIG. 7), while a less cumulative duration of presenting RTi-like symptoms was constantly observed in the probiotic group (p=0.131; FIG. 8), resulting in a significantly less cumulative days of antibiotic consumption in the probiotic group during the whole study period (p=0.011; FIG. 9). It can be seen that the cumulated days of antibiotic use prominently increased along with the cumulated duration of presenting RTi-like symptoms among patients in control group, meanwhile, no antibiotic was prescribed to the patients by practitioners in probiotic group due to that the presented RTi-like symptom was less severe, so the prescribed antibiotic courses or prophylactic antibiotic use were not necessary among patients in probiotic group during this study.

There were no oropharyngeal probiotic-related adverse events reported throughout the study period, all 7 patients in the probiotic group survived and with no signs of BSI, distant metastasis or recurrent lymphoma during the following 24 months after this study was completed, indicating that oropharyngeal probiotic administration is safe for patients whose hematopoietic system are well reconstructed post-HSCT.

Prior to allo-HSCT or auto-HSCT, the recipient must undergo a chemotherapy or radiation therapy pretreatment to remove their hematopoietic stem cells and eliminate as many remaining leukemia cells as feasible in order to facilitate the successful implantation during HSCT, however, the chemotherapy or radiation therapy result in inhibited immune system, microbiome dysbiosis, bacterial translocation and proinflammatory cytokine responses. Common sources of inflammation among HSCT patients include bacterial, viral and fungal infections, as well as chronic Inflammatory/autoimmune/neurological diseases. Antibiotic prophylaxis to protect HSCT recipients from infections has prolonged their survival but its long-term consequences remained controversial for decades (Sepkowitz 29 (., Bone Marrow Transplant. 2002), 367-371; Tabarraee et al., Iran J Pharm Res. 15(Suppl) (2016), 159-163; César-Arce et al., Transplant Proc. 49 (2017), 1444-1448; Wang et al., J Microbiol Immunol Infect. 51 (2018), 123-131; Kimura et al., J Infect. 69 (2014), 13-25. It was also reported that antibiotic exposure is unrelated to immediate post-transplant infectious complications but confers a long-term adverse effect on overall survival post-HSCT (Gromowsky et al., Blood 140 (Supplement 1) (2022), 7716-7717; Mokhtar et al., Nutrients. 14 (2022), 112. Similarly, the judicious use of appropriate antifungal treatment remains a crucial part of the treatment protocol.

During the present study, it was shown that the adjuvant treatment of oropharyngeal probiotic can effectively reduce the prevalence of RTi in patients post-HSCT, shorten the duration of respiratory symptoms, and reduce the days of antibiotic use.

### Example 3: Streptococcus salivarius ENT-K12 Promotes the Healing of Chemotherapy Induced Oral Mucositis in Mice

After having successfully shown that administration of the probiotic S. salivarius ENT-K12 results in a significant advantage to delay the onset of RIOM, greatly reduced the prevalence of severe RIOM (Example 1) and fully protected patients that underwent HSCT from ulcerative OM (Example 2), the effect of S. salivarius ENT-K12 on chemotherapy induced oral mucositis and in particular on the epithelial layer was further analyzed in mouse studies. In addition, studies have been performed to show the effect of heat-killed S. salivarius ENT-K12_on oral epithelial cells that were damaged due to the administration of chemotherapy.

Initial studies have been performed with a mouse model comprising chemotherapy treatment with busulfan and cyclophosphamide as described in the following.

### Experimental Grouping: (40 Male C57BL/6 Mice Aged 6-8 Weeks)

1) The blank control group (n=10): no intervention;
2) Disease injury group (n=10): busulfan+cyclophosphamide combination chemotherapy;
3) Probiotic intervention group A (n=10): busulfan+cyclophosphamide+heat-killed probiotic intervention;
4) Probiotic intervention group B (n=10): busulfan+cyclophosphamide+live probiotic intervention.

### Materials:

DMSO (for dissolving busulfan and cyclophosphamide), 4% paraformaldehyde, 0.1% heparin, PBS, pentobarbital sodium, busulfan, cyclophosphamide, probiotic tablets, heat-killed probiotic, 100 μl pipette, 1 ml syringe, etc.

Prepare reagent: weigh 100 mg of busulfan dissolved in 25 ml of DMSO to make up a 4 mg/ml solution for use; dissolve 0.25 g of cyclophosphamide in 25 ml of DMSO to make a 10 mg/ml solution for further use. All the above solutions are wrapped in tin foil and stored in cold storage in the dark.

### Mode of Administration and Dosage

i) Busulfan+cyclophosphamide combination chemotherapy: Intraperitoneal injection: 1 injection at a fixed time per day. The weight needs to be weighed before injection, and the post-administration was calculated (administration dose: busulfan (BU) used a dose of 80 mg/kg, cyclophosphamide (CY) used a dose of 200 mg/kg).
ii) Live probiotic intervention: The probiotic solution was prepared by dissolving probiotic tablets (the study product as described in Examples 1 and 2), and 100 μl probiotic solution was sucked with a pipette and applied to the mouse mouth from the 1st day to the 6th day. After drug delivery, mice were deprived of food and water for the next 30 minutes to keep the probiotics in the oral cavity for as long as possible. The blank control group was applied to 100 μl saline as placebo by the same method. All mice will be sacrificed on the 7th day.
ii) Heat-killed probiotic intervention: Take it orally as above.

### Chemotherapy Administration Time Span (First 4 Days of Busulfan and Second 2 Days of Cyclophosphamide Injection)

| Day 1 | Day 2 | Day 3 | Day 4 | Day 5 | Day 6 |
|---|---|---|---|---|---|
| BU-80 | BU-80 | BU-80 | BU-80 | CY-200 | CY-200 |

### Tissue Collection

At the assigned time point (on the 7th day after treatment of mice), they were euthanized using sodium pentobarbital. After euthanasia, animals were perfused with 0.1M phosphate-buffered saline (PBS) mixed with 0.1% heparin, followed by 4% paraformaldehyde mixed with PBS to immediately fix tongue tissues. After anatomy, tongues were dissected and fixed in the 4% paraformaldehyde solution for 3 hrs before blocking the tongue into three sections. The tissues were then incubated through a series of sucrose concentrations (0.5, 1.0 and 1.5 M) for cryo-protection before embedding in OCT compound for storage at −80° C. Tissue sections were cut at 12 m thickness and mounted directly onto slides. These slides were stored at −80° C. until processing.

### Detection Indicators

### HE-Stained

The morphological changes of taste buds were observed.

Steps: (1) Dewax the paraffin sections to water. Place the slices in xylene I for 10 min, xylene II for 10 min, anhydrous ethanol I for 5 min, anhydrous ethanol II for 5 min, 95% alcohol for 5 min, 90% alcohol for 5 min, 80% alcohol for 5 min, 70% alcohol for 5 min, and distilled water for washing. (2) Hematoxylin staining of cell nuclei. Sections were stained with Harris hematoxylin for 3-8 min, washed with tap water, differentiated with 1% hydrochloric acid alcohol for several seconds, rinsed with tap water, returned to blue with 0.6% ammonia water, and rinsed with running water. (3) Eosin-stained cytoplasm. Sections were stained in eosin staining solution for 1-3 min. (4) Dehydration and sealing. Put the slices into 95% alcohol I for 5 minutes, 95% alcohol II for 5 minutes, anhydrous ethanol I for 5 minutes, anhydrous ethanol II for 5 minutes, xylene I for 5 minutes, and xylene II for 5 minutes. (5) Microscopic examination, image acquisition and analysis.

### Toluidine Blue Staining

Tongues were stained with 1% toluidine blue in 10% acetic acid for 1 minute, followed by repeated washes with acetic acid, to reveal surface erosive or ulcerative lesions (Muanza et al., Clin Cancer Res 11 (2005), 5121). The percentage of toluidine blue positive surface area (excluding excision trauma) was calculated using the Image J software. Tissues were then fixed in 4% (v/v) paraformaldehyde solution in PBS for 2 hours at 4° C., and processed for paraffin or OCT embedding. Epithelial thickness in tongues and esophagi, and villus length in the jejunum were measured in H&E stained tissues using Image J. Three mice per group, with 3 fields per sample and 5 measurements per field, were analyzed, in a blinded fashion. Images were obtained using a Zeiss Axio Imager M1 microscope and an EC-Plan-Neofluar 920-NA 0.5 air-objective and using the AXIOVISION-SE64 Rel. 4.9.1 program (Bertolini et al., Transl Oncol 10 (2017), 612-620).

### Immunohistochemical Staining on Ki67

To compare the effects of probiotic intervention on cell proliferation, and it is a protein expressed by cells in all phases of cell cycle except G0 and early G1.

Steps: For this procedure, slides were first washed in PBS, then bathed in 10 mM sodium citrate (pH 6.0) at 95° C. for 15 min to induce antigen retrieval before cooling to room temperature. After washes in PBS, the tissues were blocked with a mix of 5% NGS, 1% BSA and 0.3% Triton-X 100 for 1 hr at room temperature. The rabbit anti-Ki67 primary antibody was added to the tissues, then covered with hybristrips for incubation overnight at 40° C. The following day sections were washed in PBS before they were incubated for two hrs in Alexa 546 goat-anti-rabbit secondary antibody at room temperature. Sections were then washed in PB before bathed in Sytox green to double label nuclei. After additional washes, cover slips were mounted using Fluoromount G (Delay et al., PLoS One 14 (2019), e0214890; Mukherjee et al., PLoS One 12 (2017), e0185473; Mukherjee et al., PLoS One 8 (2013), e61607).

### E-Cadherin Detection

Testing the integrity of adherens junctions in oral mucosa.

Steps: Paraffin embedded (for E-cadherin) or frozen (for PMN) tissue sections were stained with an anti-E-cadherin polyclonal antibody followed by a FITC-conjugated secondary antibody, or for PMN with the monoclonal antibody NIMP-R14, highly specific for murine Ly-6G and Ly-6C followed by a secondary anti-rat antibody conjugated with Alexa 555. To visualize all cells the nuclear stain Hoechst 33,258 was used (Bertolini et al., Transl Oncol 10 (2017), 612-620).

### PLCβ2 Staining

Immunofluorescent labeling of PLCβ2 was used to identify type II cells in taste buds, and immunolabeling of type II cells was used to determine whether differentiated and mature taste bud cells were affected by probiotic intervention.

Steps: Slides were washed in PBS and then incubated with 5% NGS in blocking solution for 1.5 hr at room temperature. The tissues were then incubated in rabbit anti-PLCβ2 primary antibody at 1:1000 dilution overnight at 40° C. Alexa 546 goat-anti-rabbit secondary antibody (1:1000) was used to incubate the tissues for 2 hrs in the dark. Sytox green was used as a nuclear marker (Delay et al., PLoS One 14 (2019), e0214890; Mukherjee et al., PLoS One 12 (2017), e0185473; Mukherjee et al., PLoS One 8 (2013), e61607; Sarkar et al., Chem Senses (2021), 46).

### SNAP-25 Staining

Immunofluorescent labeling of SNAP-25 was used to identify type III cells in taste buds, and immunolabeling of type III cells was used to determine whether differentiated mature taste bud cells were affected by probiotic intervention.

Steps: The protocol for SNAP-25 was the same as for PLCβ2. Tissues were incubated in rabbit anti-SNAP-25 primary antibody overnight at 40° C. The concentrations, times of treatment of the Alexa 546 secondary antibody, and Sytox labeling procedures were the same as above (Delay et al., PLoS One 14 (2019), e0214890; Mukherjee et al., PLoS One 12 (2017), e0185473; Sarkar et al., Chem Senses (2021), 46).

### RNA Extraction and Reverse-Transcription Quantitative Polymerase Chain Reaction (RT-qPCR)

IL-6, IL-8, IL-1β, TNF-α, NF-kB and other pro-inflammatory cytokines in tongue were detected.

Steps: Mouse tongues were homogenized using a POLYTRON homogenizer, and the supernatants were beat by zirconia beads with phenol: chloroform: isoamyl alcohol. RNA was purified using the QIAgen RNeasy® Mini Kit and concentrations/quality were determined using a NanoDrop device. Complementary DNA was synthesized with SuperScriptIII Cells Direct® cDNA Synthesis kits. Reverse-transcription quantitative polymerase chain reaction was performed with a Bio-Rad CFX96 cycler and the iQ® SYBR Green Supermix (Bertolini et al., Transl Oncol 10 (2017), 612-620).

### Statistical Analyses

Immunofluorescent images were captured using a color camera mounted on a Nikon Eclipse E600 Scope and Spot acquisition software. Cell counts were done by observers blind to the experimental conditions using the criteria of Nguyen et al. Images were enhanced as needed to more clearly identify immuno-positive cells in Adobe Photoshop CS6 (https://www.Adobe.com) by adjusting brightness and RGB levels before quantification using NIH ImageJ (https://imagej.nih.gov/ij). The percentage score for Ki67 was calculated by dividing the number of immuno-positive cells for Ki67+ cells by the total number of basal epithelial cells or Sytox-positive taste sensory cells. Only cells within the basement layer and within the walls of the crypt of the circumvallate papilla that contained taste buds were counted. The data for PLCβ2 and SNAP-25 labeling were means of 5-12 taste buds per mouse. The number of immune-positive cells and the total number of nuclei labeled with Sytox green were counted within each taste bud. The data for all experiments were collected from 3 to 6 mice per group.

Linear model analyses of variance (ANOVA) were used to analyze the data for Ki67, PLCβ2, and SNAP-25. Separate ANOVAs were performed to evaluate counts of labeled cells, total number of cells within taste buds, and the percentage of labeled cells within taste buds for each cellular label. All statistical tests were performed with SPSS version 26.0. Graphs were made with GraphPad Prism 8 (Delay et al., PLoS One 14 (2019), e0214890; Mukherjee et al., PLoS One 12 (2017), e0185473; Mukherjee et al., PLoS One 8 (2013), e61607).

### Results

9 mice were dosed at a dose of 80 mg/kg busulfan, and 3 mice were observed to die one day later. Accordingly, subsequent experiments were performed with busulfan at a total dose of 120 mg/kg administered in 4 divided days at 30 mg/kg and cyclophosphamide at a total dose of 200 mg/kg administered in 2 divided days at 100 mg/kg.

Chemotherapy administration time span (first 4 days of busulfan+second 2 days of cyclophosphamide injection) is shown below:

| Day 1 | Day 2 | Day 3 | Day 4 | Day 5 | Day 6 |
|---|---|---|---|---|---|
| BU-30 | BU-30 | BU-30 | BU-30 | CY-100 | CY-100 |

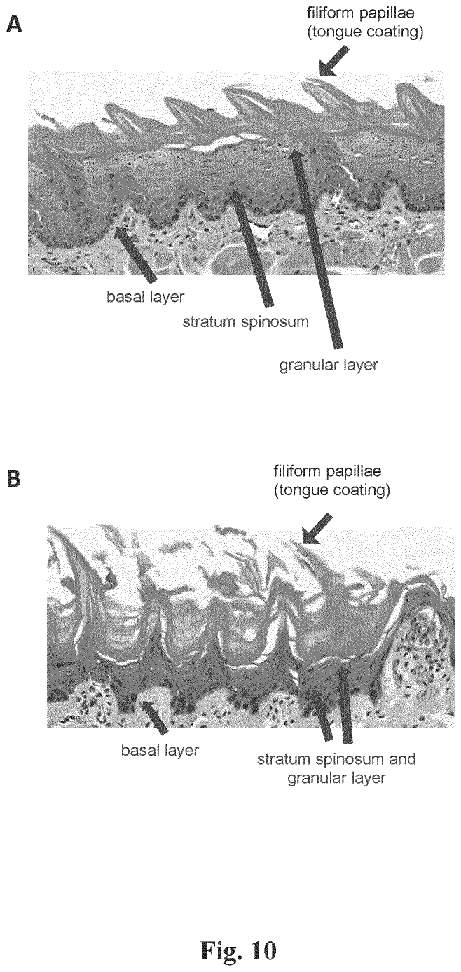

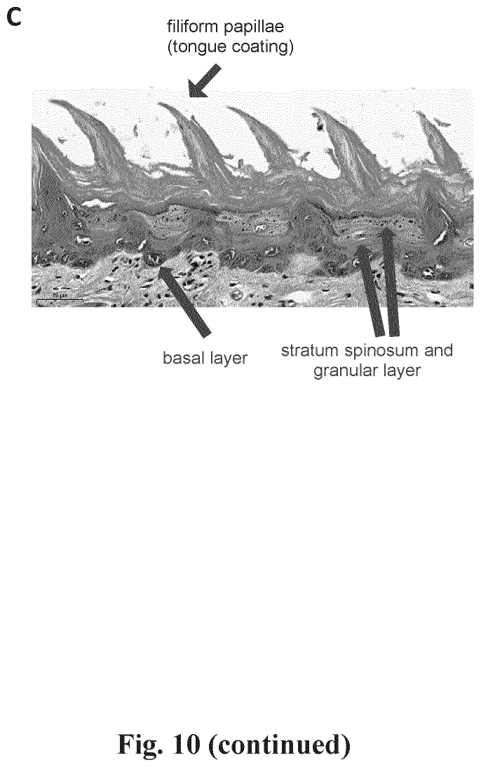

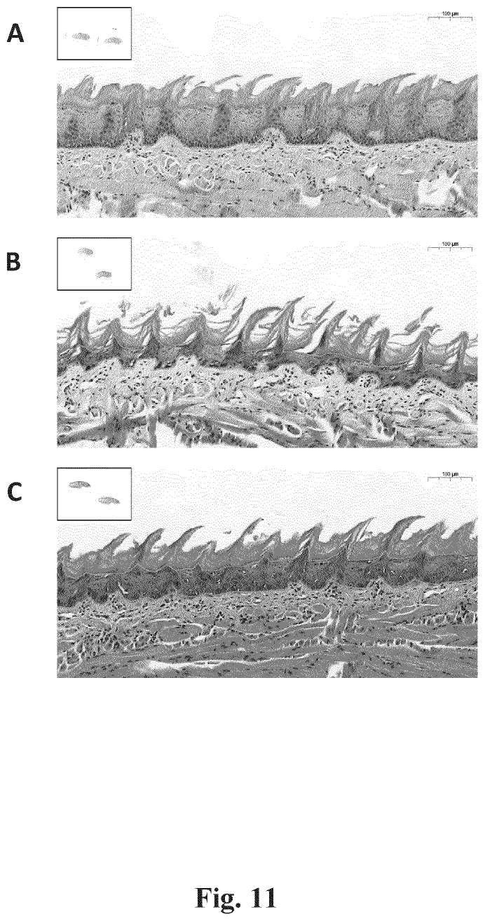

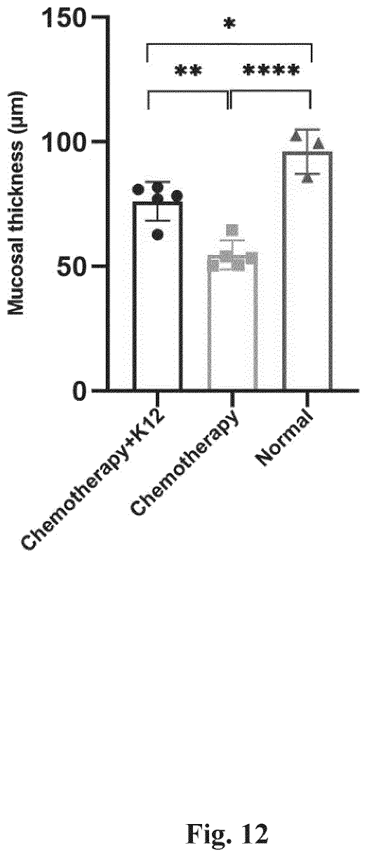

First results showed that Streptococcus thermophilus ENT-K12 promotes the healing of chemotherapy induced oral mucositis in mice. In particular, chemotherapy treatment in mice led to conspicuous mucosal hypoplasia and ulceration in the tongue. Furthermore, the basal layer cells were only loosely aligned and pyknosis, an irreversible condensation of chromatin in the nucleus of a cell undergoing necrosis or apoptosis, was observed. In addition, the tongue tissue had less stratum spinosum and granular layer cell than healthy tissue; see FIG. 10A and FIG. 10B. Treatment with ENT-K12 during chemotherapy restored the integrity of the lingual mucosa and partially restored the basal layer, stratum spinosum and granular layer; see FIG. 10C. It was further observed that chemotherapy treatment reduced the mucosal thickness by nearly 50% and that the reduction was less, i.e., about 25% less when ENT-12 was administered during chemotherapy; see FIGS. 11 A to C, and FIG. 12.

Before the conduction of further experiments, the mouse model was switched to a mouse model comprising administering 5-fluorouracil (5-Fu) because this chemotherapeutic agent is more commonly used during chemotherapy in human patients; see Example 4

### Example 4: Live and Inactivated Streptococcus salivarius ENT-K12 Promote the Healing of Chemotherapy Induced Oral Mucositis in Mice

In the present experiment, the effect of the chemotherapeutic agent 5-fluorouracil (5-Fu) and the administration of S. salivarius ENT-K12 on 5-Fu damaged oral epithelial cells was analyzed.

### Material and Methods:

Preparation of the reagents: 5 mg of 5-Fu was dissolved in 100 μl DMSO solvent to form 5 mg/100 μl (50 μg/μl) of 5-Fu protosolution. Since DMSO is cytotoxic, 10 μl of 5-Fu protosolution was diluted in 10 ml DMEM medium to form a 1000-fold dilution. That is, the solution concentration is 50 μg/ml. In the intervention group, it is not only necessary to form a 5-Fu solution of 50 μg/ml, but also to add the heat-killed S. salivarius ENT-K12 solution and heat-killed S. salivarius ENT-K12 supernatant.

Method for preparing heat-killed S. salivarius ENT-K12 solution: Heat inactivation was performed by incubation of S. salivarius for 60 min at 65° C.

### Establishment of the Animal Model:

Eighteen 8-week-old mice with a body weight of about 20 g were selected and assigned to the control group, the chemotherapy group and the probiotic intervention group after chemotherapy (n=6). Mice in the 5-FU group and the probiotic intervention group were injected with 5-FU at a dose of 50 mg/kg for 5 consecutive days. Mice in the intervention group were given oral irrigation twice a day with probiotics S. salivarius ENT-K12 of 1*10⁹ CFU collected by centrifuge, while those in the 5-Fu group were given oral irrigation twice a day with sterile pure water.

Tissue collection and staining was done as described in Example 3.

### Analysis of the Growth and Proliferative Activity of Oral Epithelial Cells (HOK Cells) in the Presence of 5-Fu

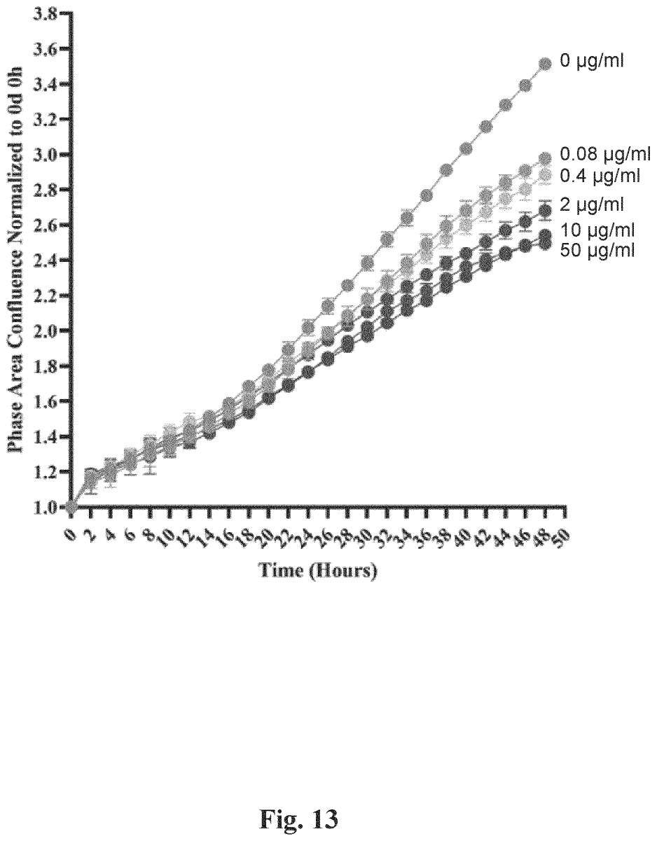

In a first set of experiments, the growth activity of oral epithelial cells (HOK cells) in the presence of 5-Fu was analyzed. For this, 5-Fu was added to the 96-well plate in 6 different concentrations, 0, 0.08, 0.4, 2, 10, and 50 μg/mL, and each concentration was set to 6 wells, and 5000 HOK cells were added to each well. The HOK cells were incubated in a carbon dioxide incubator for 24 hours after the panel. Cell proliferation was detected by live cell analysis (LCA). The instrument was set up to take a picture once every 2 hours, for a total of 2 days, 5 pairs of wells in each group, 4 fields of view in each well, and 10× shooting. As shown by a life cell imaging experiment and visualized in FIG. 13, the growth activity of HOK cells decrease after 48 hours of 5-Fu treatment.

Furthermore, the proliferative activity of HOK cells was analyzed. Again, 5-Fu was added to the 96-well plate in 6 different concentrations, 0, 0.08, 0.4, 2, 10, and 50 μg/mL, and each concentration was set to 6 wells, and 5000 HOK cells were added to each well. The cells in the 96-well plates were treated with 5-Fu for 48 hours and afterwards, 10 μl of CCK8 reagent was added to each well. CCK8 refers to the Cell Counting Kit-8 which allows for a sensitive colorimetric assay for the determination of cell viability in cell proliferation and cytotoxicity assays, as for example available at MedChemExpress (Article number: HY-K0301).

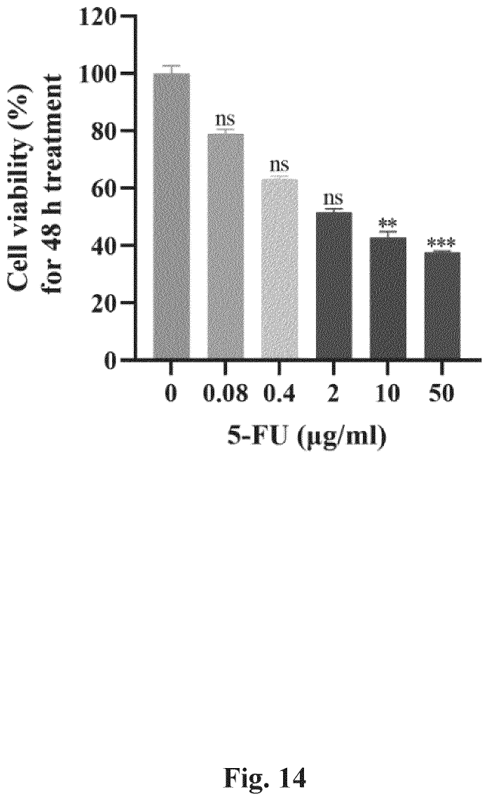

After 4 hours of treatment, the cells were detected by a standard microplate reader (for example Multiskan™ FC microplate photometer of Thermo Scientific). The mitochondrial metabolic concentration was measured to reflect cell proliferation activity and a significantly decreased proliferation activity of HOK cells was induced by 5-FU treatment with concentration >10 μg/mL for 48 hours; see FIG. 14.

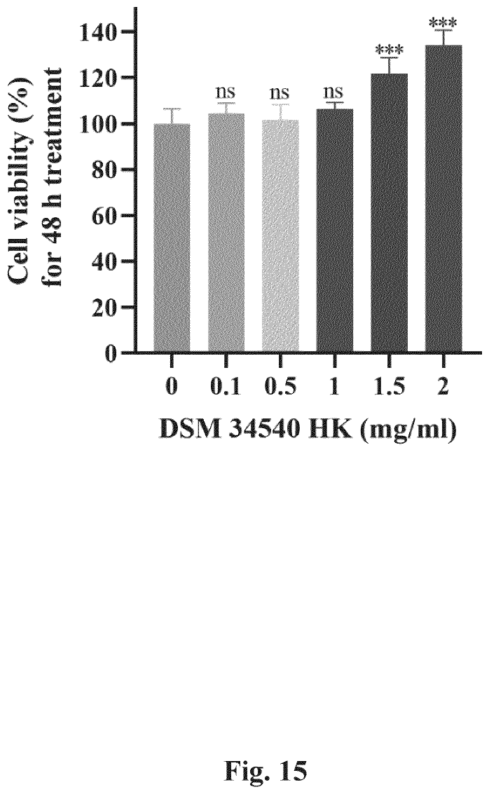

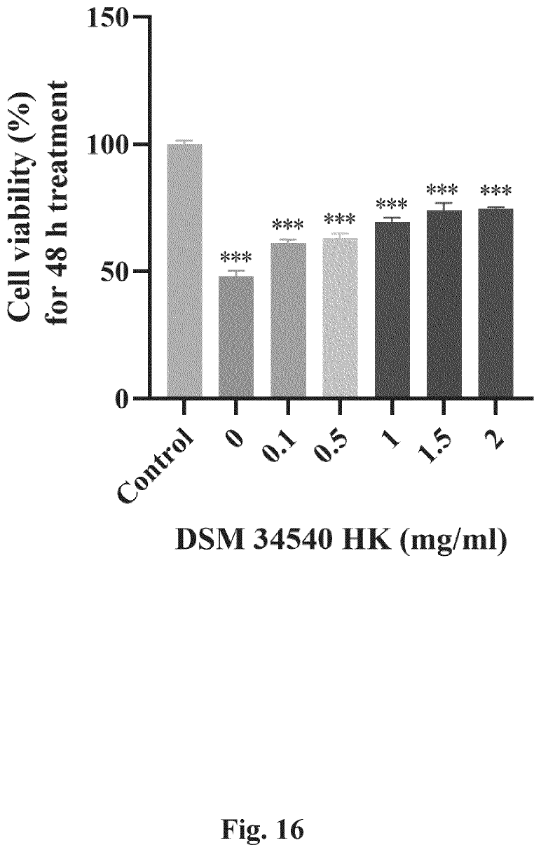

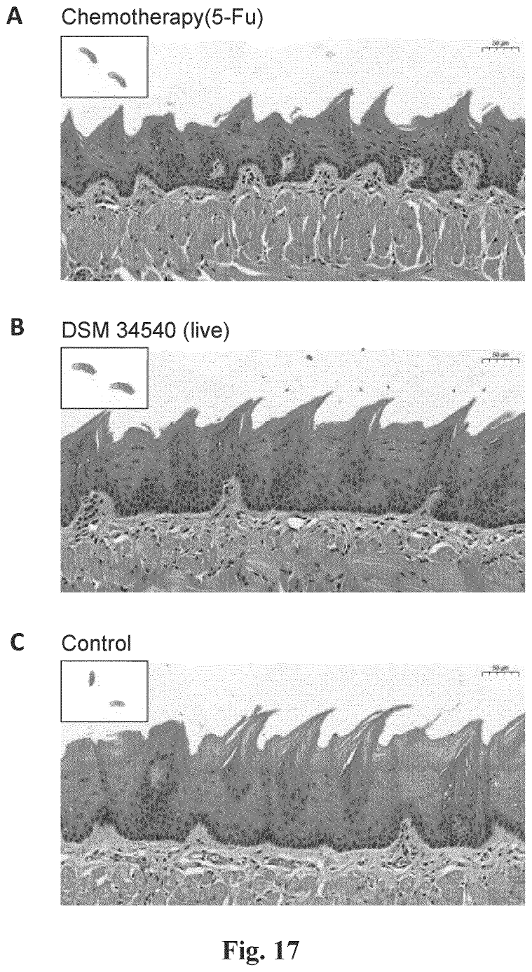

Effect of heat-killed S. salivarius ENT-K12 on proliferation of oral epithelial cells (HOK cells) A solution of heat-killed S. salivarius ENT-K12 was prepared as described above and added in different concentrations ranging from 0 mg/mL to 2 mg/mL to HOK cells in 96-well plates (5000 HOK cells per well) to determine the effect of heat-killed S. salivarius ENT-K12 on their proliferation. Incubation was performed for 48 hours and afterwards, 101 of CCK8 reagent was added to each well. After 4 hours of treatment, the cells were detected by a microplate reader. As visualized in FIG. 15, heat-killed S. salivarius ENT-K12 showed no proliferative toxicity to HOK cells at any concentration, and the cell viability of HOK was significantly increased by heat-killed DSM 34540 treatment when the concentration was >1.5 mg/mL. Furthermore, the proliferation capacity of HOK cells was analyzed in the presence of 5-Fu and the effect of S. salivarius ENT-K12 on the HOK cells. For this, a solution of heat-killed S. salivarius ENT-K12 was prepared as described above and added in different concentrations ranging from 0 mg/mL to 2 mg/mL to HOK cells in 96-well plates (5000 HOK cells per well). Furthermore, 10 μg/mL 5-Fu was added to each well. Incubation was performed for 48 hours and afterwards, 10 μl of CCK8 reagent was added to each well. After 4 hours of treatment, the cells were detected by a microplate reader. As shown in FIG. 16, the proliferation of HOK cells significantly decreased after 10 μg/mL 5-Fu treatment. However, the treatment of the HOK cells with heat-killed S. salivarius ENT-K12, restored their proliferation significantly and dose-dependently.

Protection of Oral Mucosal Barrier In Vivo by Live and Heat Killed S. salivarius ENT-K12

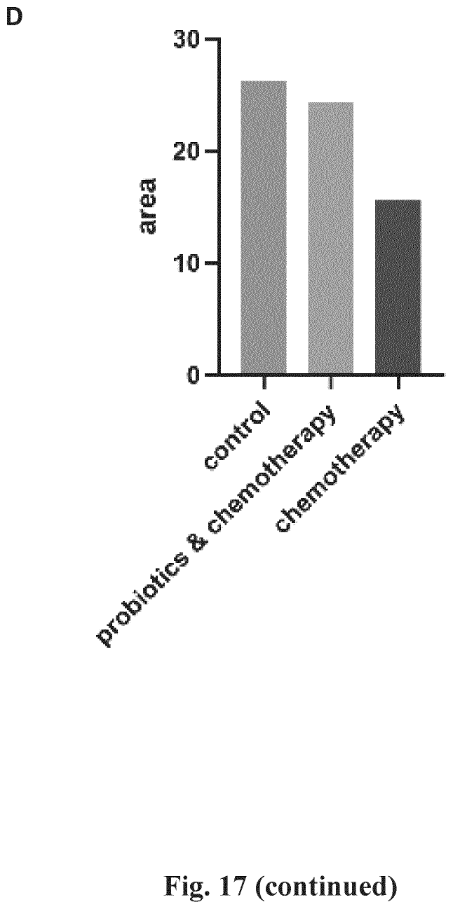

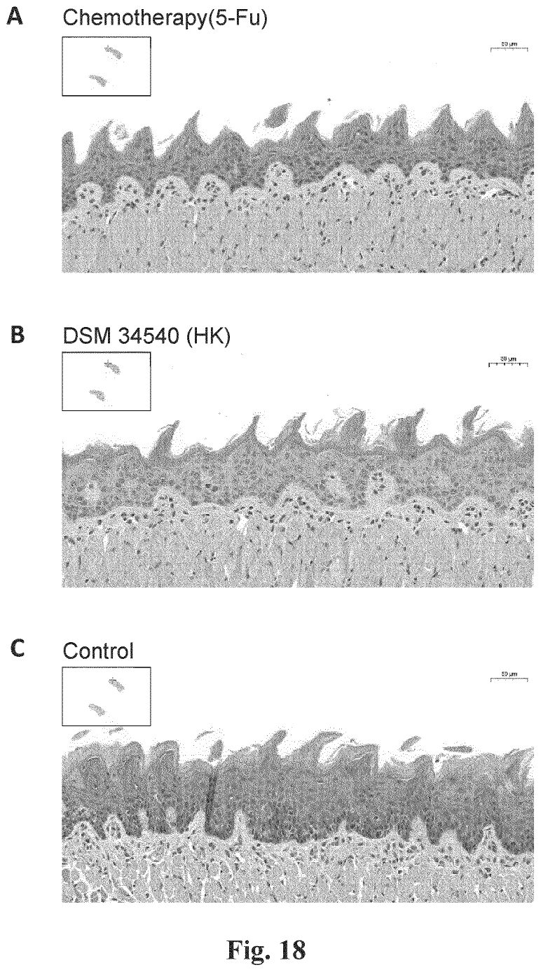

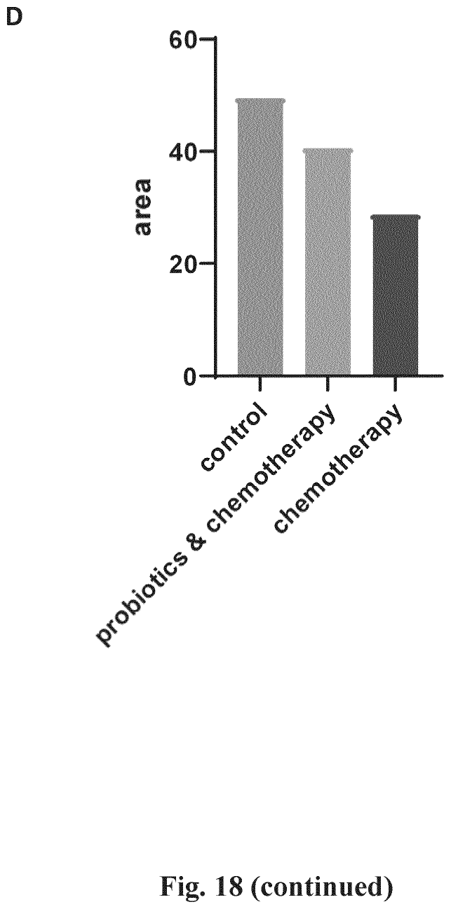

As shown in FIGS. 17A and 17D as well as in FIGS. 18A and 18D, 5-Fu had a significant adverse effect on the integrity and permeability of the oral mucosal barrier of mice. As clearly visible, the area of oral mucosal layer of mice was reduced by 5-Fu treatment and significantly increased by both live and heat-killed S. salivarius ENT-K12 treatment.

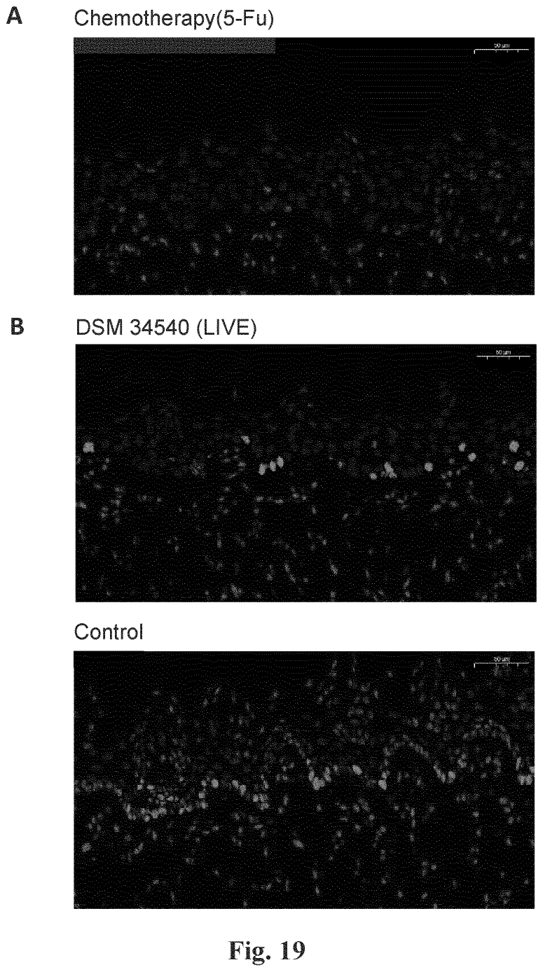

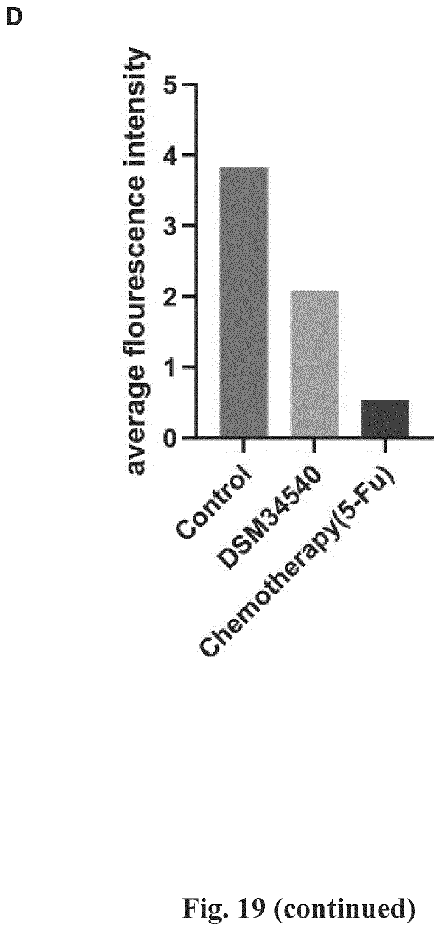

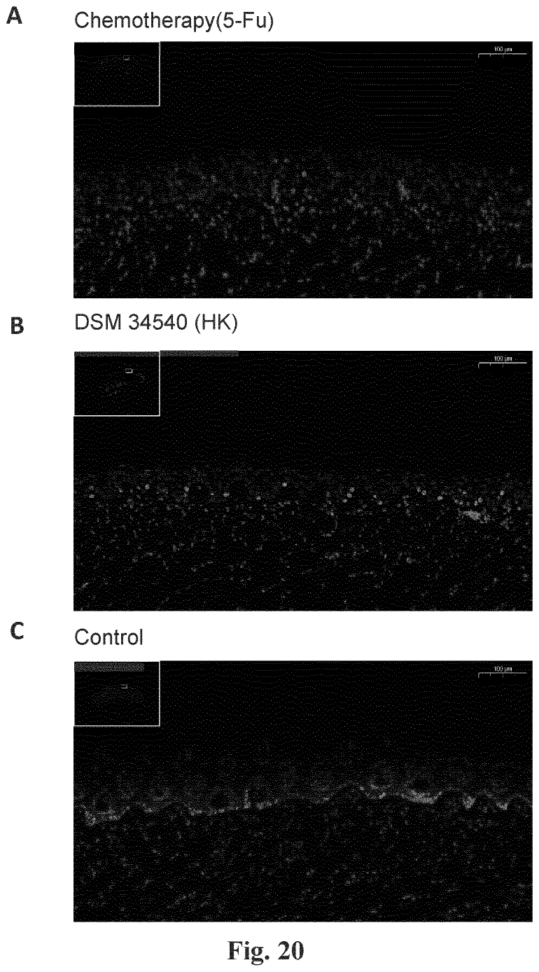

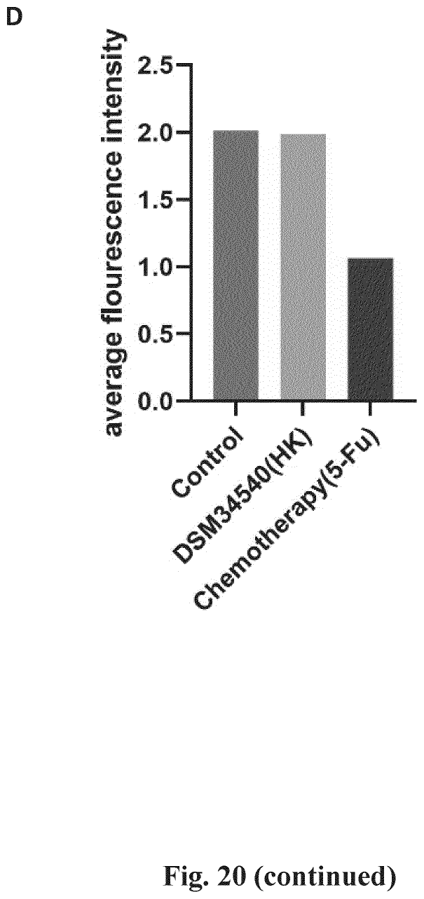

Furthermore, the expression of Ki-67 was analyzed. Ki-67 is a protein which is present in the nucleus and is only expressed during phase of cell division, which is associated with cell proliferation. As shown in FIGS. 19 and 20, the expression of Ki-67 in the 5-Fu group was lower than that in the probiotic group and the control group. Thus, both live and heat-killed S. salivarius ENT-K12 significantly promoted the proliferation of oral mucosa cells.

Heat-Treated S. salivarius ENT-K12 Improved the Damage of HOK Cell Cycle Induced by Chemotherapy 5-Fu

The cell cycle was analyzed by flow cytometry. For this, a solution of heat-killed S. salivarius ENT-K12 was prepared and CCK-8 analysis as well as detection of cell proliferation by LCA was performed as also described above.

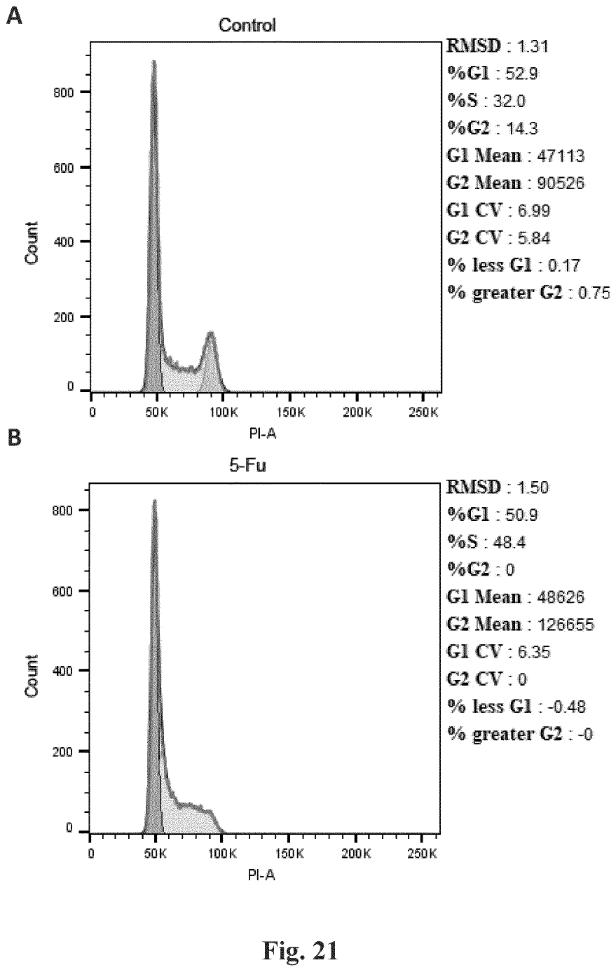

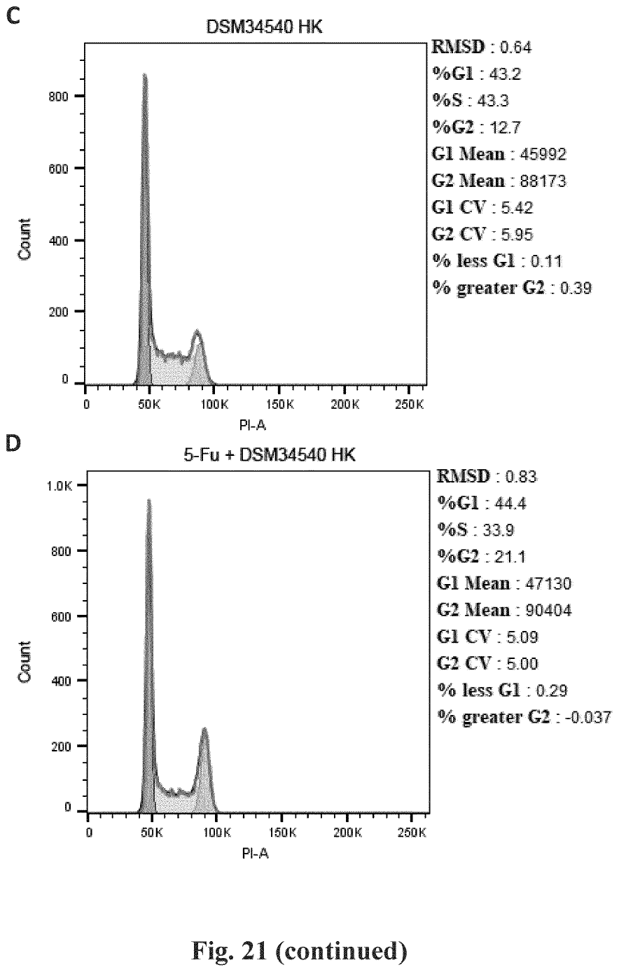

The results are visualized in FIG. 21 and show that compared to the control group, heat-killed S. salivarius ENT-K12 did not significantly change the HOK cell cycle after 48-hour treatment, whereas 5-Fu treatment induced the block of S phase and G2/M phase, which are associated with a period of rapid cell growth and protein synthesis during which the cell prepares itself for mitosis and cytokinesis. Remarkably, heat-killed S. salivarius ENT-K12_restored the whole cell cycle after it was disturbed due to 5-Fu treatment.

S. salivarius ENT-K12 Reduced the DNA Damage Level of HOK Cell Induced by Chemotherapy 5-Fu

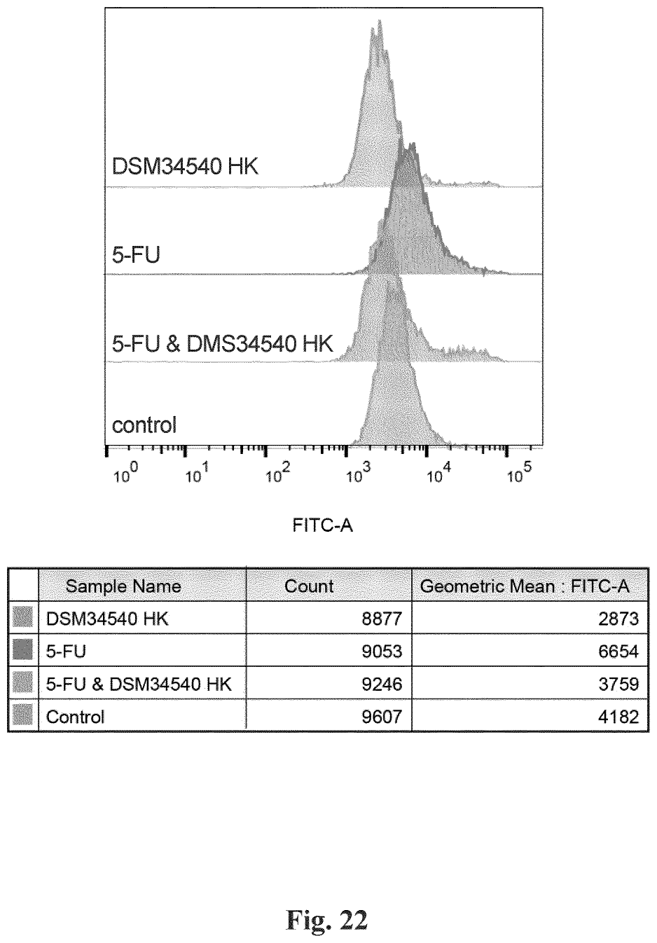

In a further experiment, the reactive oxygen species (ROS) level of HOK cell was detected by flow cytometry as described above. As shown in FIG. 22, the ROS level of HOK cells, which usually reflects the DNA damage during chemotherapy, was induced by 5-Fu treatment, and was significantly reduced after the treatment of heat-killed S. salivarius ENT-K12.

In summary, it was shown that heat-killed S. salivarius ENT-K12 has a beneficial effect on chemotherapy damaged oral epithelial cells and thus, can—next to live S. salivarius ENT-K12—be used for the prevention and treatment of oral mucositis.

1. Streptococcus salivarius for use in the treatment or prevention of an ear, nose, and throat (ENT) disease which is induced by or associated with a cancer therapy.

2. Streptococcus salivarius for use according to claim 1, wherein the ENT disease is a cancer therapy-induced oral mucosa disorder.

3. Streptococcus salivarius for use according to claim 1, wherein the ENT disease is a cancer therapy-induced respiratory tract infection.

4. Streptococcus salivarius for use according to any one of claims 1 to 3, wherein the ENT disease is induced by or associated with radiotherapy, chemotherapy, hematopoietic stem cell transplantation (HSCT), immune checkpoint inhibitor therapy, or any combination thereof.

5. Streptococcus salivarius for use according to any one of claims 1 to 4, wherein the ENT disease is induced by or associated with radiotherapy or concurrent chemoradiation therapy (CCRT).

6. Streptococcus salivarius for use according to any one of claims 1 to 4, wherein the ENT disease is induced by or associated with chemotherapy.

7. Streptococcus salivarius for use according to any one of claims 1 to 4, wherein the ENT disease is induced by or associated with hematopoietic stem cell therapy (HSCT).

8. Streptococcus salivarius for use according to any one of claims 1 to 4, wherein the ENT disease is indued by or associated with HSCT which is preceded by chemotherapy.

9. Streptococcus salivarius use according to any one of claims 1 to 4, wherein the ENT disease is induced by or associated with treatment of a head and neck cancer, preferably of a nasopharyngeal carcinoma (NPC) by radiotherapy or CCRT.

10. Streptococcus salivarius for use according to any one of claims 1 to 4, wherein the ENT disease is induced by or associated with treatment of a hematopoietic tumor by HSCT, preferably by chemotherapy followed by HSCT.

11. Streptococcus salivarius for use in the treatment or prevention of an ear, nose, and throat (ENT) disease, which is induced by or associated with an immunosuppression, preferably wherein the ENT disease is an oral mucosa disorder or a respiratory tract infection.

12. Streptococcus salivarius for use according to any one of claims 1 to 11, wherein the oral mucosa disorder is oral mucositis.

13. Streptococcus salivarius for use according to any one of claims 1, 2 and 4 to 12, wherein Streptococcus salivarius is provided in an inactive form, preferably wherein Streptococcus salivarius is heat inactivated.

14. Streptococcus salivarius for use according to claim 12 or 13, wherein administration of Streptococcus salivarius
(i) delays the onset of oral mucositis and reduces the risk of prevalence of severe oral mucositis;
(ii) restores the decreased proliferation and the disturbed cell cycle of oral epithelial cells after chemotherapy treatment in a dose-dependent manner; and/or
(iii) prevents the reduction of the oral mucosal layer area and restores the disturbed integrity of the oral mucosa after chemotherapy treatment.

15. Streptococcus salivarius for use according to claim 3, wherein administration of Streptococcus salivarius
(i) prevents the progression of an upper respiratory tract infection to a lower respiratory tract infection in the subject;
(ii) shortens the duration of symptoms of a respiratory tract infection in the subject in comparison to a control, which did not administer Streptococcus salivarius;
(iii) reduces the average duration of the respiratory tract infection episode in the subject in comparison to the control group; and/or
(iv) reduces the need of antibiotic consumption of the subject in comparison to the control group.

16. Streptococcus salivarius for use according to any one of claims 1 to 12 and 14 to 15, wherein Streptococcus salivarius is orally administered at a daily dose between 3×10⁶ and 4×10¹⁰ CFU, preferably between 3×10⁹ and 3×10¹⁰ CFU or between 4×10⁹ and 4×10¹⁰ CFU.

17. Streptococcus salivarius for use according to any one of claims 1 to 12 and 14 to 16, wherein Streptococcus salivarius is orally administered at a daily dose between 3×10 mg and 4×120 mg, preferably 3×50 mg or 4×50 mg of Streptococcus salivarius.

18. Streptococcus salivarius for use according to any one of claims 1 to 17, wherein Streptococcus salivarius is orally administered 4 times a day for a period of about 6 to 7 weeks to a subject during radiotherapy or chemoradiation therapy.

19. Streptococcus salivarius for use according to claim 18, wherein Streptococcus salivarius is orally administered 4 times a day for a period of about 2 weeks before commencement of the radiotherapy or chemoradiation therapy.

20. Streptococcus salivarius for use according to any one of claims 1 to 17, wherein Streptococcus salivarius is orally administered 3 times a day for a period of about 100 days to a subject which has undergone HSCT and after the hematopoietic system of the subject is reconstituted which is defined as blood platelets >20*10⁹ cells/L and neutrophil >0.5*10⁹ cells/L after HSCT.

21. Streptococcus salivarius for use according to any one of claims 1 to 20, wherein Streptococcus salivarius is formulated as a composition, preferably an oral composition.

22. Streptococcus salivarius for use according to any one of claims 1 to 21, wherein the composition is an oral dosage form selected from a tablet, a capsule, a gel, a lozenge, a chewable tablet, an oil drop, and a powder, preferably wherein the composition is an oil drop or a solid oral dosage form selected from a tablet, a capsule, a chewable tablet and a lozenge, most preferably a lozenge.

23. Streptococcus salivarius for use according to claim 22, wherein the oral dosage form comprises at least 10⁶ CFU of Streptococcus salivarius, preferably between 10⁶ and 10¹⁰ CFU of Streptococcus salivarius, more preferably 10⁹ or 10¹⁰ CFU of Streptococcus salivarius.

24. Streptococcus salivarius for use according to claim 22 or 23, wherein the oral dosage form comprises 1 to 500 mg, preferably 1 to 120 mg, preferably 10 to 120 mg of Streptococcus salivarius, most preferably 50 mg of Streptococcus salivarius.

25. Streptococcus salivarius for use according to any one of claims 21 to 24, wherein the oral dosage form is a lozenge.

26. Streptococcus salivarius for use according to claim 26, wherein the lozenge further comprises fructose, maltodextrin, magnesium stearate and a flavor, preferably strawberry flavor.

27. Streptococcus salivarius for use according to claim 25 or 36, wherein the lozenge is a slow dissolving lozenge, preferably wherein the disintegration time of the lozenge is at least 4 to 5 minutes.

28. Streptococcus salivarius for use according to any one of claims 25 to 27, wherein the lozenge comprises about 50 mg or 5% Streptococcus salivarius, about 635 mg or 63.5% fructose, about 290 mg or 29% maltodextrin, about 15 mg or 1.5% magnesium stearate, and about 10 mg or 1% flavor.

29. A composition comprising Streptococcus salivarius for use in the treatment or prevention of an ear, nose, and throat (ENT) disease which is induced by or associated with a cancer therapy.

30. The composition for use according to claim 29, wherein the ENT disease is a cancer therapy-induced oral mucosa disorder.

31. The composition for use according to claim 29, wherein the ENT disease is a cancer therapy-induced respiratory tract infection.

32. The composition for use according to any one of claims 29 to 31, wherein the ENT disease is induced by or associated with radiotherapy, chemotherapy, hematopoietic stem cell transplantation (HSCT), immune checkpoint inhibitor therapy, or any combination thereof.

33. The composition for use according to any one of claims 29 to 32, wherein the ENT disease is induced by or associated with treatment of
(i) a head and neck cancer, preferably of a nasopharyngeal carcinoma (NPC) by radiotherapy or chemoradiation therapy, or
(ii) a hematopoietic tumor by HSCT, preferably by chemotherapy followed by HSCT.

34. A composition comprising Streptococcus salivarius for use in the treatment or prevention of an ear, nose, and throat (ENT) disease, which is induced by or associated with an immunosuppression, preferably wherein the ENT disease is an oral mucosa disorder or a respiratory tract infection.

35. The composition for use according to any one of claims 29 to 34, wherein the oral mucosa disorder is oral mucositis.

36. The composition for use according to any one of claims 29, 30 and 32 to 35, wherein the composition comprises inactivated, preferably heat-inactivated Streptococcus salivarius.

37. The composition for use according to any one of claims 29 to 36, wherein the composition is an oral composition.

38. The composition for use according to any one of claims 29 to 37, wherein the composition is an oral dosage form selected from a tablet, a capsule, a gel, a lozenge, a chewable tablet, an oil drop, and a powder, preferably wherein the composition is an oil drop or a solid oral dosage form selected from a tablet, a capsule, a chewable tablet and a lozenge, most preferably a lozenge.

39. The composition for use according to any one of claims 29 to 35 and 37 to 38, wherein the composition comprises at least 10⁶ CFU of Streptococcus salivarius, preferably between 10⁶ and 10¹⁰ CFU of Streptococcus salivarius, more preferably 10⁹ or 10¹⁰ CFU of Streptococcus salivarius.

40. The composition for use according to any one of claims 29 to 35 and 37 to 39, wherein the composition comprises 1 to 500 mg, preferably 1 to 120 mg, preferably 10 to 120 mg of Streptococcus salivarius, most preferably 50 mg of Streptococcus salivarius.

41. The composition for use according to any one of claims 29 to 35 and 37 to 40, wherein the composition comprises 50 mg Streptococcus salivarius, and wherein the composition is orally administering several times a day, preferably 3 times a day, or 4 times a day.

42. The composition for use according to any one of claims 29 to 35 and 37 to 41, wherein the composition comprises at least 10⁶ CFU of Streptococcus salivarius, and wherein the composition is orally administered several times a day, preferably 3 times a day, or 4 times a day.

43. The composition for use according to any one of claims 29 to 42, wherein the composition is orally administered 4 times a day for a period of about 6 to 7 weeks to a subject during radiotherapy or chemoradiation therapy.

44. The composition for use according to claim 43, wherein the composition is orally administered 4 times a day for a period of about 2 weeks before commencement of the radiotherapy or chemoradiation therapy.

45. The composition for use according to any one of claims 29 to 42, wherein the composition is orally administered 3 times a day for a period of about 100 days to a subject which has undergone HSCT and after the hematopoietic system of the subject is reconstituted which is defined as blood platelets >20*10⁹ cells/L and neutrophil >0.5*10⁹ cells/L after HSCT.

46. The composition for use according to any one of claims 29 to 45, wherein the composition is a lozenge, preferably a lozenge as defined in any one of claims 26 to 28.

47. Use of a composition comprising Streptococcus salivarius in the maintenance of healthy oral mucosa of a subject during treatment with a cancer or autoimmune disease therapy, during treatment with radiotherapy, chemotherapy, hematopoietic stem cell transplantation (HSCT), immune checkpoint inhibitor therapy, or any combination thereof, or of a subject which is immunosuppressed.

48. The use according to claim 47, wherein the composition is the composition as defined in any one of claims 37 to 40 and 43 to 46.

49. Streptococcus salivarius or a composition comprising Streptococcus salivarius for use in the treatment or prevention of a respiratory tract infection in a human subject, wherein the human subject is immunosuppressed.

50. Streptococcus salivarius or the composition for use according to claim 49, wherein the immunosuppression is caused by cancer or autoimmune disease treatment.

51. Streptococcus salivarius or the composition for use according to claim 49 or 50, wherein the cancer treatment comprises HSCT and/or chemotherapy, preferably HSCT preceded by chemotherapy.

52. Streptococcus salivarius or the composition for use according to any one of claims 49 to 51, wherein administration of Streptococcus salivarius prevents the progression of an upper respiratory tract infection to a lower respiratory tract infection in the subject.

53. Streptococcus salivarius or the composition for use according to any one of claims 49 to 52, wherein administration of Streptococcus salivarius shortens the duration of symptoms of a respiratory tract infection in the subject in comparison to a control, which did not administer Streptococcus salivarius or a composition comprising Streptococcus salivarius.

54. Streptococcus salivarius or the composition for use according to any one of claims 49 to 53, wherein administration of Streptococcus salivarius reduces the average duration of the respiratory tract infection episode in the subject in comparison to the control group.

55. Streptococcus salivarius or the composition for use according to any one of claims 49 to 54, wherein administration of Streptococcus salivarius reduces the need of antibiotic consumption of the subject in comparison to the control group.

56. Streptococcus salivarius for use according to any one of claims 49 to 55, wherein Streptococcus salivarius is formulated as a composition, preferably an oral composition.

57. The composition for use according to any one of claims 49 to 55, wherein the composition is an oral composition.

58. Streptococcus salivarius or the composition for use according to any one of claims 49 to 57, wherein the composition is an oral dosage form selected from a tablet, a capsule, a gel, a lozenge, a chewable tablet, an oil drop, and a powder, preferably wherein the composition is a lozenge.

59. Streptococcus salivarius or the composition for use according to any one of claims 49 to 58, wherein the composition comprises at least 10⁶ CFU of Streptococcus salivarius, preferably between 10⁶ and 10¹⁰ CFU of Streptococcus salivarius, more preferably 10⁹ or 10¹⁰ CFU of Streptococcus salivarius.

60. Streptococcus salivarius or the composition for use according to any one of claims 49 to 59, wherein the composition comprises 1 to 500 mg, preferably 1 to 120 mg, preferably 10 to 120 mg of Streptococcus salivarius, most preferably 50 mg of Streptococcus salivarius.

61. Streptococcus salivarius or the composition for use according to any one of claims 49 to 60, wherein Streptococcus salivarius is orally administered at a daily dose between 3×10⁶ and 4×10¹⁰ CFU, preferably between 3×10⁹ and 3×10¹⁰ CFU to the subject.

62. Streptococcus salivarius or the composition for use according to any one of claims 49 to 61, wherein Streptococcus salivarius is orally administered a daily dose between 3×10 mg and 4×120 mg, preferably 3×50 mg to the subject.

63. Streptococcus salivarius or the composition for use according to any one of claims 49 to 60, wherein the composition comprises 50 mg Streptococcus salivarius, and wherein the composition is orally administering several times a day, preferably 3 times a day to the subject.

64. Streptococcus salivarius or the composition for use according to any one of claims 49 to 60, wherein the composition comprises at least 10⁶ CFU of Streptococcus salivarius, and wherein the composition is orally administered several times a day, preferably 3 times a day to the subject.

65. Streptococcus salivarius or the composition for use according to any one of claims 49 to 64, wherein the composition is orally administered 3 times a day for a period of about 100 days to a subject which has undergone HSCT and after the hematopoietic system of the subject is reconstituted which is defined as blood platelets >20*10⁹ cells/L and neutrophil >0.5*10⁹ cells/L after HSCT.

66. Streptococcus salivarius or the composition for use according to any one of claims 59 to 65, wherein the composition is an oral solid dosage form, preferably a lozenge, most preferably a lozenge as defined in any one of claims 26 to 28.

67. A method of treating or preventing an ear, nose, throat (ENT) disease which is induced by or associated with a cancer therapy in a subject in need thereof, wherein the method comprises administering Streptococcus salivarius or a composition comprising Streptococcus salivarius to the subject.

68. The method of claim 67, wherein the ENT disease is a cancer therapy-induced oral mucosa disorder or a cancer therapy-associated respiratory tract infection.

69. The method of claim 67 or 68, wherein the ENT disease is the ENT disease as defined in any one of claims 2 to 12.

70. The method of any one of claims 67 to 69, wherein Streptococcus salivarius is Streptococcus salivarius as defined in any one of the preceding claims.

71. The method of any one of claims 67 to 69, wherein the composition is the composition as defined in any one of the preceding claims.

72. The method of any one of claims 67 to 71, wherein Streptococcus salivarius and the composition, respectively is administered as defined in any one of the preceding claims.

73. Use of Streptococcus salivarius or a composition comprising Streptococcus salivarius in the manufacturing of a medicament for treating or preventing an ear, nose, throat (ENT) disease which is induced by or associated with a cancer therapy in a subject in need thereof.

74. The use of claim 73, wherein the ENT disease is a cancer therapy induced oral mucosa disorder or a cancer therapy associated respiratory tract infection, preferably wherein the ENT disease is the ENT disease as defined in any one of claims 2 to 12.

75. The use of claim 73 or 74, wherein Streptococcus salivarius is Streptococcus salivarius as defined in any one of the preceding claims.

76. The use of claim 73 or 74, wherein the composition is the composition as defined in any one of the preceding claims.

77. The use of any one of claims 73 to 76, wherein Streptococcus salivarius and the composition, respectively is administered as defined in any one of the preceding claims.

78. A method of treating or preventing a respiratory tract infection in a human subject, wherein the human subject is immunosuppressed, the method comprising administering Streptococcus salivarius or a composition comprising Streptococcus salivarius to the subject.

79. The method of claim 78, wherein the subject is the subject as defined in claim 50 or 51.

80. The method of claim 78 or 79, wherein Streptococcus salivarius is Streptococcus salivarius as defined in any one of claims 56 and 58 to 66.

81. The method of claim 78 or 79, wherein the composition is the composition as defined in any one of claims 57 to 66.

82. The method of any one of claims 78 to 81, wherein Streptococcus salivarius and the composition, respectively is administered as defined in any one of claims 61 to 65.

83. A lozenge comprising Streptococcus salivarius for use in the treatment of an ear, nose, throat (ENT) disease which is induced by or associated with a cancer therapy, wherein the ENT disease is a cancer therapy induced oral mucosa disorder or a cancer therapy associated respiratory tract infection, preferably wherein the ENT disease as defined in any one of claims 1 to 12.

84. A lozenge comprising Streptococcus salivarius for use in the treatment of a respiratory tract infection in a human subject, wherein the human subject is immunosuppressed and preferably defined as in claim 50 or 51.

85. The lozenge of claim 83 or 84, which is the lozenge as defined in any one of claims 26 to 28, and is preferably administered as defined in any one of the preceding claims.

86. Streptococcus salivarius for use according to any one of claims 1 to 28, 49 to 56, and 58 to 66, a composition for use according to any one of claims 29 to 46, 49 to 55, and 57 to 66, the use of any one of claim 47 or 48, and 73 to 77, the method of any one of claims 67 to 72, and 78 to 82, or the lozenge of any one of claims 83 to 85, wherein Streptococcus salivarius is Streptococcus salivarius K12 (American Type Culture Collection (ATCC), P.O. Box 1549, Manassas, VA 20108, USA, Accession No. BAA-1024) or Streptococcus salivarius ENT-K12 (Leibniz-Institut DSMZ—Deutsche Sammlung von Mikroorganismen und Zellkulturen GmbH, Inhoffenstralße 7B, 38124 Braunschweig, Germany, Accession No. DSM 34540).
